SEQUENCE LISTING (78 sequences)

SEQ ID NO: 1 - DNA, 23 bp, synthetic construct
  source 1..23: mol_type = other DNA; note = Cre forward primer
gcggtctggcagtaaaaactatc

SEQ ID NO: 2 - DNA, 23 bp, synthetic construct
  source 1..23: mol_type = other DNA; note = Cre reverse primer
gtgaaacagcattgctgtcactt

SEQ ID NO: 3 - DNA, 20 bp, synthetic construct
  source 1..20: mol_type = other DNA; note = LSL-Cas9 wt forward primer
aagggagctgcagtggagta

SEQ ID NO: 4 - DNA, 18 bp, synthetic construct
  source 1..18: mol_type = other DNA; note = LSL-Cas9 wt reverse primer
caggacaacgcccacaca

SEQ ID NO: 5 - DNA, 18 bp, synthetic construct
  source 1..18: mol_type = other DNA; note = LSL-Cas9 mt forward primer
tccccatcaagctgatcc

SEQ ID NO: 6 - DNA, 20 bp, synthetic construct
  source 1..20: mol_type = other DNA; note = LSL-Cas9 mt reverse primer
cttcttctttggggccatct

SEQ ID NO: 7 - DNA, 26 bp, synthetic construct
  source 1..26: mol_type = other DNA; note = Npy-GFP common forward primer
tatgtggacggggcagaagatccagg

SEQ ID NO: 8 - DNA, 24 bp, synthetic construct
  source 1..24: mol_type = other DNA; note = Npy-GFP wt reverse primer
cccagctcacatatttatctagag

SEQ ID NO: 9 - DNA, 20 bp, synthetic construct
  source 1..20: mol_type = other DNA; note = Npy-GFP mt reverse primer
ggtgcggttgccgtactgga

SEQ ID NO: 10 - DNA, 19 bp, synthetic construct
  source 1..19: mol_type = other DNA; note = Pomc-GFP forward primer
aagttcatctgcaccaccg

SEQ ID NO: 11 - DNA, 20 bp, synthetic construct
  source 1..20: mol_type = other DNA; note = Pomc-GFP reverse primer
tgctcaggtagtggttgtcg

SEQ ID NO: 12 - DNA, 20 bp, synthetic construct
  source 1..20: mol_type = other DNA; note = CRISPR mediated deletion of mouse InsR forward primer
gagatggtccacctgaagga

SEQ ID NO: 13 - DNA, 20 bp, synthetic construct
  source 1..20: mol_type = other DNA; note = CRISPR mediated deletion of mouse InsR reverse primer
gtgaaggtcttggcagaagc

SEQ ID NO: 14 - RNA, 20 nt, synthetic construct
  source 1..20: mol_type = other RNA
  ncRNA 1..20: ncRNA_class = guide_RNA; note = Scramble guide RNA
  modified_base 19: mod_base = OTHER; note = thymine
  modified_base 2: mod_base = OTHER; note = thymine
  modified_base 4: mod_base = OTHER; note = thymine
  modified_base 7: mod_base = OTHER; note = thymine
  modified_base 8: mod_base = OTHER; note = thymine
  modified_base 15: mod_base = OTHER; note = thymine
  modified_base 16: mod_base = OTHER; note = thymine
gtgtagttcgaccattcgtg

SEQ ID NO: 15 - RNA, 20 nt, synthetic construct
  source 1..20: mol_type = other RNA
  ncRNA 1..20: ncRNA_class = guide_RNA; note = Insr guide RNA #1
  modified_base 18: mod_base = OTHER; note = thymine
  modified_base 1: mod_base = OTHER; note = thymine
  modified_base 3: mod_base = OTHER; note = thymine
  modified_base 8: mod_base = OTHER; note = thymine
  modified_base 11: mod_base = OTHER; note = thymine
  modified_base 16: mod_base = OTHER; note = thymine
tatcgactggtcccgtatcc

SEQ ID NO: 16 - RNA, 20 nt, synthetic construct
  source 1..20: mol_type = other RNA
  ncRNA 1..20: ncRNA_class = guide_RNA; note = Insr guide RNA #2
  modified_base 6: mod_base = OTHER; note = thymine
  modified_base 2: mod_base = OTHER; note = thymine
  modified_base 4: mod_base = OTHER; note = thymine
gtctgtccaggcaccgccaa

SEQ ID NO: 17 - DNA, 48 bp, synthetic construct
  source 1..48: mol_type = other DNA
ggggacaactttgtatagaaaagttggagggcctatttcccatgattc

SEQ ID NO: 18 - DNA, 48 bp, synthetic construct
  source 1..48: mol_type = other DNA
ggggactgcttttttgtacaaacttgaaaaaagcaccgactcggtgcc

SEQ ID NO: 19 - DNA, 48 bp, synthetic construct
  source 1..48: mol_type = other DNA
atatcttgtggaaaggacgaaacaccgtatcgactggtcccgtatccg

SEQ ID NO: 20 - DNA, 45 bp, synthetic construct
  source 1..45: mol_type = other DNA
aacttgctatttctagctctaaaacttggcggtgcctggacagac

SEQ ID NO: 21 - DNA, 23 bp, synthetic construct
  source 1..23: mol_type = other DNA; note = Adamst4 forward primer
gaacggtggcaagtattgtgagg

SEQ ID NO: 22 - DNA, 22 bp, synthetic construct
  source 1..22: mol_type = other DNA; note = Adamst4 reverse primer
ttcggtggttgtaggcagcaca

SEQ ID NO: 23 - DNA, 23 bp, synthetic construct
  source 1..23: mol_type = other DNA; note = Adamst5 forward primer
ctgccttcaaggcaaatgtgtgg

SEQ ID NO: 24 - DNA, 22 bp, synthetic construct
  source 1..22: mol_type = other DNA; note = Adamst5 reverse primer
caatggcggtaggcaaactgca

SEQ ID NO: 25 - DNA, 20 bp, synthetic construct
  source 1..20: mol_type = other DNA; note = Il-6 forward primer
ggtgccctgccagtattctc

SEQ ID NO: 26 - DNA, 19 bp, synthetic construct
  source 1..19: mol_type = other DNA; note = Il-6 reverse primer
ggctcccaacacaggatga

SEQ ID NO: 27 - DNA, 23 bp, synthetic construct
  source 1..23: mol_type = other DNA; note = Kcna4 forward primer
gcagattgctgaatgacacctcg

SEQ ID NO: 28 - DNA, 23 bp, synthetic construct
  source 1..23: mol_type = other DNA; note = Kcna4 reverse primer
ggacaagcaaagcatcgaaccac

SEQ ID NO: 29 - DNA, 22 bp, synthetic construct
  source 1..22: mol_type = other DNA; note = Kcnb1 forward primer
gaggagttcgacaacacgtgct

SEQ ID NO: 30 - DNA, 22 bp, synthetic construct
  source 1..22: mol_type = other DNA; note = Kcnb1 reverse primer
tgagtgacagggcaatggtgga

SEQ ID NO: 31 - DNA, 22 bp, synthetic construct
  source 1..22: mol_type = other DNA; note = Kcnb2 forward primer
gctggagaaacctaactcgtcc

SEQ ID NO: 32 - DNA, 23 bp, synthetic construct
  source 1..23: mol_type = other DNA; note = Kcnb2 reverse primer
ctcgtcgttttcttgcagctctg

SEQ ID NO: 33 - DNA, 24 bp, synthetic construct
  source 1..24: mol_type = other DNA; note = Kcnc3 forward primer
gaagaggtgattgaaaccaacagg

SEQ ID NO: 34 - DNA, 22 bp, synthetic construct
  source 1..22: mol_type = other DNA; note = Kcnc3 reverse primer
tgggctcttgtcttctggagac

SEQ ID NO: 35 - DNA, 22 bp, synthetic construct
  source 1..22: mol_type = other DNA; note = Kcnc4 forward primer
ccagctcgaatcgcccatttac

SEQ ID NO: 36 - DNA, 22 bp, synthetic construct
  source 1..22: mol_type = other DNA; note = Kcnc4 reverse primer
agcaccgcattagcatcgccat

SEQ ID NO: 37 - DNA, 22 bp, synthetic construct
  source 1..22: mol_type = other DNA; note = Kcnd2 forward primer
cctacatgcagagcaagcggaa

SEQ ID NO: 38 - DNA, 23 bp, synthetic construct
  source 1..23: mol_type = other DNA; note = Kcnd2 reverse primer
gtggttttctccaggcagtgaag

SEQ ID NO: 39 - DNA, 22 bp, synthetic construct
  source 1..22: mol_type = other DNA; note = Kcnd3 forward primer
agaagaggagcagatgggcaag

SEQ ID NO: 40 - DNA, 23 bp, synthetic construct
  source 1..23: mol_type = other DNA; note = Kcnd3 reverse primer
cttgatggtggaggttcgtacag

SEQ ID NO: 41 - DNA, 22 bp, synthetic construct
  source 1..22: mol_type = other DNA; note = Kcnj11 forward primer
tgcgtcacaagcatccactcct

SEQ ID NO: 42 - DNA, 22 bp, synthetic construct
  source 1..22: mol_type = other DNA; note = Kcnj11 reverse primer
ggacattcctctgtcaccatgc

SEQ ID NO: 43 - DNA, 22 bp, synthetic construct
  source 1..22: mol_type = other DNA; note = Kcnj3 forward primer
cagttcgaggttgtcgtcatcc

SEQ ID NO: 44 - DNA, 22 bp, synthetic construct
  source 1..22: mol_type = other DNA; note = Kcnj3 reverse primer
cccaaagcacttcgtcctctgt

SEQ ID NO: 45 - DNA, 23 bp, synthetic construct
  source 1..23: mol_type = other DNA; note = Kcnj6 forward primer
ggaactggagattgtggtcatcc

SEQ ID NO: 46 - DNA, 22 bp, synthetic construct
  source 1..22: mol_type = other DNA; note = Kcnj6 reverse primer
tcttccagcgttaggacaggtg

SEQ ID NO: 47 - DNA, 22 bp, synthetic construct
  source 1..22: mol_type = other DNA; note = Kcnj9 forward primer
tctcacctctcgtcatcagcca

SEQ ID NO: 48 - DNA, 22 bp, synthetic construct
  source 1..22: mol_type = other DNA; note = Kcnj9 reverse primer
gcttcgagcttggcacgtcatt

SEQ ID NO: 49 - DNA, 23 bp, synthetic construct
  source 1..23: mol_type = other DNA; note = Kcnma1 forward primer
cctgaaggactttctgcacaagg

SEQ ID NO: 50 - DNA, 22 bp, synthetic construct
  source 1..22: mol_type = other DNA; note = Kcnma1 reverse primer
actccacctgagtgaaatgccg

SEQ ID NO: 51 - DNA, 22 bp, synthetic construct
  source 1..22: mol_type = other DNA; note = Kcnn3 forward primer
tccaccgtcatcctgcttggtt

SEQ ID NO: 52 - DNA, 22 bp, synthetic construct
  source 1..22: mol_type = other DNA; note = Kcnn3 reverse primer
caggctgatgtagaggatacgc

SEQ ID NO: 53 - DNA, 22 bp, synthetic construct
  source 1..22: mol_type = other DNA; note = Kcnq3 forward primer
aagcctacgctttctggcagag

SEQ ID NO: 54 - DNA, 22 bp, synthetic construct
  source 1..22: mol_type = other DNA; note = Kcnq3 reverse primer
acagctcggatggcagccttta

SEQ ID NO: 55 - DNA, 22 bp, synthetic construct
  source 1..22: mol_type = other DNA; note = Mmp13 forward primer
agcagttccaaaggctacaact

SEQ ID NO: 56 - DNA, 20 bp, synthetic construct
  source 1..20: mol_type = other DNA; note = Mmp13 reverse primer
ggatgcttagggttggggtc

SEQ ID NO: 57 - DNA, 20 bp, synthetic construct
  source 1..20: mol_type = other DNA; note = Mmp14 forward primer
agcactgggtgtttgacgaa

SEQ ID NO: 58 - DNA, 21 bp, synthetic construct
  source 1..21: mol_type = other DNA; note = Mmp14 reverse primer
ccggtagtacttattgccccg

SEQ ID NO: 59 - DNA, 20 bp, synthetic construct
  source 1..20: mol_type = other DNA; note = Mmp2 forward primer
gtcgcccctaaaacagacaa

SEQ ID NO: 60 - DNA, 20 bp, synthetic construct
  source 1..20: mol_type = other DNA; note = Mmp2 reverse primer
ggtctcgatggtgttctggt

SEQ ID NO: 61 - DNA, 22 bp, synthetic construct
  source 1..22: mol_type = other DNA; note = Mmp9 forward primer
gctgactacgataaggacggca

SEQ ID NO: 62 - DNA, 22 bp, synthetic construct
  source 1..22: mol_type = other DNA; note = Mmp9 reverse primer
tagtggtgcaggcagagtagga

SEQ ID NO: 63 - DNA, 19 bp, synthetic construct
  source 1..19: mol_type = other DNA; note = r18s forward primer
cagctccaagcgttcctgg

SEQ ID NO: 64 - DNA, 23 bp, synthetic construct
  source 1..23: mol_type = other DNA; note = r18s reverse primer
ggccttcaattacagtcgtcttc

SEQ ID NO: 65 - DNA, 21 bp, synthetic construct
  source 1..21: mol_type = other DNA; note = Tgf beta 1 forward primer
ggataccaactattgcttcag

SEQ ID NO: 66 - DNA, 20 bp, synthetic construct
  source 1..20: mol_type = other DNA; note = Tgf beta 1 reverse primer
tgtccaggctccaaatatag

SEQ ID NO: 67 - DNA, 23 bp, synthetic construct
  source 1..23: mol_type = other DNA; note = Tgf beta 2 forward primer
ctaatgttgttgccctcctacag

SEQ ID NO: 68 - DNA, 23 bp, synthetic construct
  source 1..23: mol_type = other DNA; note = Tgf beta 2 reverse primer
gcacagaagttagcattgtaccc

SEQ ID NO: 69 - DNA, 23 bp, synthetic construct
  source 1..23: mol_type = other DNA; note = Tgf beta r1 forward primer
ggaccattgtgttacaagaaagc

SEQ ID NO: 70 - DNA, 23 bp, synthetic construct
  source 1..23: mol_type = other DNA; note = Tgf beta r1 reverse primer
catggcgtaacattacagtctga

SEQ ID NO: 71 - DNA, 23 bp, synthetic construct
  source 1..23: mol_type = other DNA; note = Tgf beta r2 forward primer
tcctagtgaagaacgacttgacc

SEQ ID NO: 72 - DNA, 23 bp, synthetic construct
  source 1..23: mol_type = other DNA; note = Tgf beta r2 reverse primer
taccagagccatggagtagacat

SEQ ID NO: 73 - DNA, 22 bp, synthetic construct
  source 1..22: mol_type = other DNA; note = Timp1 forward primer
tcttggttccctggcgtactct

SEQ ID NO: 74 - DNA, 23 bp, synthetic construct
  source 1..23: mol_type = other DNA; note = Timp1 reverse primer
gtgagtgtcactctccagtttgc

SEQ ID NO: 75 - DNA, 22 bp, synthetic construct
  source 1..22: mol_type = other DNA; note = Timp3 forward primer
gctagaagtcaacaaataccag

SEQ ID NO: 76 - DNA, 21 bp, synthetic construct
  source 1..21: mol_type = other DNA; note = Timp3 reverse primer
tagtagcaggacttgatcttg

SEQ ID NO: 77 - DNA, 20 bp, synthetic construct
  source 1..20: mol_type = other DNA; note = Tnf alpha forward primer
ctgtgaagggaatgggtgtt

SEQ ID NO: 78 - DNA, 20 bp, synthetic construct
  source 1..20: mol_type = other DNA; note = Tnf alpha reverse primer
ggtcactgtcccagcatctt

(19) United States
(12) Patent Application Publication
(10) Pub. No.: US 2026/0191933 A1
(43) Pub. Date: Jul. 9, 2026

(54) PHARMACEUTICAL COMBINATIONS AND USES THEREOF

(71) Applicant: The University of Melbourne, The University of Melbourne, Victoria (AU)

(72) Inventors: Garron DODD, The University of Melbourne, Victoria (AU); Cait Anwen BEDDOWS, The University of Melbourne, Victoria (AU); Feiyue SHI, The University of Melbourne, Victoria (AU); Callen Charles LUCAS-GOLDSMITH, The University of Melbourne, Victoria (AU)

(21) Appl. No.: 19/132,984

(22) PCT Filed: Nov. 28, 2023
(86) PCT No.: PCT/AU2023/051216

§ 371 Date: May 27, 2025

(30) Foreign Application Priority Data
Nov. 28, 2022 (AU) 2022903599

(51) Int. Cl. A61K 38/26; A61K 9/00; A61K 31/7008; A61K 31/7072; A61P 3/04; A61P 3/10

(52) U.S. Cl. CPC A61K 38/26; A61K 9/0019; A61K 9/0043; A61K 31/7008; A61K 31/7072; A61P 3/04; A61P 3/10

(57) ABSTRACT

The present invention relates to pharmaceutical combinations for treating or preventing insulin resistance and associated disorders, in particular the use combinations comprising a 4-epimerase inhibitor and a GLP-1 receptor agonist for treating or preventing insulin resistance and associating disorders such as obesity and type-2 diabetes, supressing appetite and/or promoting weight loss.

### CROSS-REFERENCE TO RELATED APPLICATION

This application claims priority from Australian Provisional Patent Application No. 2022903599 filed 28 Nov. 2022, the entire contents of which are incorporated herein by cross-reference.

### TECHNICAL FIELD

The present invention relates generally to pharmaceutical combinations for treating or preventing insulin resistance and disorders associated with insulin resistance. In particular, the present invention is directed to pharmaceutical combinations comprising a 4-epimerase inhibitor and a GLP-1 receptor agonist for treating or preventing insulin resistance and associated disorders such as obesity and type-2 diabetes, supressing appetite and/or promoting weight loss.

### BACKGROUND

Metabolic diseases such as obesity and type-2 diabetes mellitus affect around 650 million people worldwide. The global prevalence of metabolic diseases has tripled since 1975 and if current trends continue without the advent of effective treatments, >51% of the world population will be obese or type-2 diabetic by 2030. A key hallmark of many metabolic diseases, including obesity and type-2 diabetes, is insulin resistance.

Insulin is a peptide hormone that is synthesised and secreted by beta cells of the pancreas. Following secretion into the blood, insulin influences a variety of cells expressed throughout the body, where it plays a critical role in maintaining blood glucose levels within an optimum range. In this context, insulin targets peripheral tissues, including the skeletal muscle and adipose tissue, to promote glucose uptake from the circulation, and the liver to represses gluconeogenesis and glycogenolysis. Insulin also signals to the brain, where it plays a vital role in how the brain controls whole-body glucose and energy homeostasis. Insulin resistance occurs when insulin's target tissues in the periphery become insensitive to the action of insulin.

Obesity is characterised by an excess accumulation of adipose tissue, which is highly responsive to insulin and contributes greatly to both glucose and lipid metabolism. In obese individuals, adipose tissue releases higher amounts of non-esterified fatty acids, glycerol, hormones, and pro-inflammatory cytokines, which are involved in the development of insulin resistance. Initially, beta (β-)cells compensate for insulin resistance by secreting more insulin, but over time beta cells cannot keep up with the body's demand for insulin and blood sugar levels become elevated. Thus, in addition to its own health complications, obesity-associated insulin resistance is also a major risk factor for type-2 diabetes.

Type-2 diabetes can be a difficult disease to manage because it requires long-term maintenance of blood glucose levels. Current antidiabetic drugs do not control blood sugar levels well enough to completely prevent the occurrence of high and low blood sugar levels, which can cause long-term complications such as retinopathy, renopathy, neuropathy and peripheral vascular disease. People with type-2 diabetes mellitus are also at increased risk of developing other conditions, such as obesity, hypertension, stroke, heart disease and hyperlipidemia.

In addition to metabolic diseases such as obesity and type-2 diabetes, insulin resistance is associated with a host of other serious health problems, including metabolic syndrome, hypertension, dyslipidemia, hyperglycemia, atherosclerosis, non-alcoholic fatty liver disease (NAFLD), polycystic ovary syndrome (PCOS) and coagulopathy.

Glucagon-like peptide 1 receptor (GLP-1R) agonists and analogues thereof, such as liraglutide, stimulate insulin secretion to improve glucose homeostasis in obese and diabetic patients by activating GLP-1 receptors in the arcuate nucleus of the hypothalamus (ARC). However, functional GLP-1 receptor signalling becomes impaired in obese patients and GLP-1 receptor agonists such as liraglutide are less effective in reducing weight and HbA1c level, an index of long-term glycaemic control, for patients with higher BMI levels. Reduced GLP-1-induced insulin release from pancreatic β-cells has also been observed in obese patients with impaired glucose tolerance.

While there are a number of GLP-1R agonists available on the market, they tend to be less effective in patients with a higher BMI. For example, following long-term administration of liraglutide, 45.9% of overweight patients (BMI<30 kg/m²) lost more than 10% of their body weight, as compared with 29.9% of patients with morbid obesity (BMI>40 kg/m²) (Pi-Sunyer, et al., 2015). Clinical efficacy of GLP-1 receptor agonists on improving glycaemic control also appears to wane over time (Davies et al., 2015). Furthermore, I.C.V. administration of GLP-1 impedes lipid uptake into white adipose tissue through the sympathetic nervous system in lean mice rather than in obese mice. Previous attempts to resolve limitations of single GLP-1R agonists have had limited success. For example, single liraglutide treatment has involved simultaneously administering insulin degludec and liraglutide, which can lead to weight gain.

Accordingly, there is a need for improved or alternative methods for treating or preventing insulin resistance and associated disorders, including metabolic diseases such as obesity and type-2 diabetes.

### SUMMARY

In one aspect, the present invention provides a pharmaceutical combination comprising (i) a 4-epimerase inhibitor and (ii) a GLP-1 receptor (GLP-1R) agonist.

In another aspect, the present invention provides a pharmaceutical composition comprising a 4-epimerase inhibitor and a GLP-1 agonist.

In another aspect, the present invention provides a method for treating or preventing insulin resistance or an associated disorder, supressing appetite and/or promoting weight loss in a subject, the method comprising administering to the subject an effective amount of a 4-epimerase inhibitor in combination with a GLP-1 receptor (GLP-1R) agonist.

In another aspect, the present invention provides use of a 4-epimerase inhibitor and a GLP-1R agonist in the manufacture of one or more medicaments for treating or preventing insulin resistance or an associated disorder, supressing appetite and/or promoting weight loss in a subject.

In another aspect, the present invention provides use of a 4-epimerase inhibitor in the manufacture of a medicament for treating or preventing insulin resistance or an associated disorder, supressing appetite and/or promoting weight loss in a subject in combination with a GLP-1R agonist.

In another aspect, the present invention provides use of a GLP-1R agonist in the manufacture of a medicament for treating or preventing insulin resistance or an associated disorder, supressing appetite and/or promoting weight loss in a subject in combination with a 4-epimerase inhibitor.

In another aspect, the present invention provides a combination comprising (i) a 4-epimerase inhibitor and (ii) a GLP-1R agonist, for use in treating or preventing insulin resistance or an associated disorder, supressing appetite and/or promoting weight loss in a subject.

### BRIEF DESCRIPTION OF THE DRAWINGS

Embodiments of the invention will now be described with reference to the following Figures, which are intended to be exemplary only, and in which:

FIG. 1: Obesity drives neurofibrosis within the ARC. Aged matched C57BL/6J mice were fed a chow or a HFHS diet for 12 weeks and brains were processed for a-c) WFA or g-i) aggrecan immunostaining, b,h) area and c,i) intensity within the ARC was quantified. d) ARC homogenates from 12-week obese or aged-matched chow fed C57BL/6J were subjected to ZIC-HILIC chromatography and CS-GAG and HA abundance was quantified using 2-aminobenzamide fluorescent labelled disaccharides from enzymatically depolymerised GAG chains. C57BL/6J mice were fed HFHS diet for 0, 3 days, 1, 4, 8 or 12 weeks and brains were processed for immunohistochemistry monitoring for e,f) WFA or m,n) aggrecan expression within the ARC; f,n) staining area was quantified. Aged matched C57BL/6J mice were fed a chow or a HFHS diet for 12 weeks and brains were processed for g-i) WFA and aggrecan immunostaining and j,k) co-expression within the ARC was quantified. Results are mean±SEM; significance and are representative of at least three independent experiments. Significance determined using b, c, d, h, i) t test and f, n) two-way ANOVA with Tukey multiple comparisons. Scale bar, 100 μm.

FIG. 2: Attenuated CSPG-ECM turnover in the ARC drives neurofibrosis during the development of metabolic disease. a) Schematic overview of the CSPG-ECM tracker technique. b,c) 8-week old C57BL6J mice received unilateral administration of WFA-biotin or saline into the ARC. 1-day post injection, brains were extracted and processed for immunohistochemistry monitoring for WFA-biotin and WFA-FITC and d) staining area within the ARC quantified. e) 12-week obese or aged matched chow C57BL6J male mice received bilateral administration of WFA-biotin into the ARC. Brains were extracted following 0 days, 1-, 3-, 5- or 10-weeks post injection, subjected to immunohistochemistry monitoring for the presence of WFA-biotin and WFA-FITC and f,g) CSPG-ECM turnover within the ARC quantified over time. h) Extracellular matrix regulation enzymes or pro-fibrotic factor gene expression was determined in the mediobasal hypothalamus from 12-week obese or aged matched chow C57BL6J male mice. Results are mean±SEM; significance determined using g) simple linear regression. Scale bar, 100 μm.

FIG. 3: Neurofibrosis occurs around AgRP neurons in the ARC. a-c, g-i) Npy-GFP and d-f) Pomc-EGP male mice were fed a HFHS diet for 0, 4 and 12 weeks and brains were processed for a-f) WFA or g-i) aggrecan immunostaining and b,e,h) staining encased cell number, and c,f,i) surrounding staining intensity was quantified. Whole cell patch clamp electrophysiology was conducted in NPY neurons of 12-week HFHS diet Npy-GFP mice following vehicle or chABC administration into the ARC. 4 days post injection the j) proportion of spontaneously firing neurons, k,l) firing frequency and m) resting membrane potential was determined. Results are mean±SEM; significance determined using b, c, h, i) one-way ANOVA with Tukey multiple comparisons, g) ANCOVA or k,m) unpaired t-test (two-tailed) or unpaired t-test (one-tailed), respectively. Electrophysiological recordings were made from 17 (vehicle) and 18 (chABC) neurons, with 4 mice per treatment group. Scale bar, 100 μm.

FIG. 4: Disassembly of neurofibrosis within the ARC promotes the remission of metabolic disease. a) C57BL/6J mice were fed a HFHS diet for 12 weeks and bilaterally injected with vehicle or chABC into the ARC to disassemble the CSPG-ECM. ARC targeting was confirmed by analysis of ARC WFA immunofluorescence (inserts in a). b) Body weights, c) adiposity. d) food intake, g) energy expenditure. h) ingWAT gross morphology, i) ingWAT histology and UCP-1 immunohistochemistry, j,k) inguinal dermal thermography, l) glucose tolerance and m) HOMA-IR were assessed. C57BL/6J mice were fed a HFHS for 12 weeks and bilaterally injected vehicle or chABC into the ARC. 1 day post intraARC injection, vehicle administered mice were pair-fed, with their daily food availability limited to that consumed of chABC treated mice, and e) body weight and f) adiposity were assessed. Hyperinsulinemic-euglycemic clamps were performed in conscious unrestrained C57BL/6J mice fed a HFHS diet for 12-weeks and bilaterally injected with vehicle or chABC into the ARC. Results are shown for n) GIR, o) basal and clamped EGP. p) Hyperinsulinemic-euglycemic clamped mice were administered a bolus of 2-DG and tissue specific insulin-stimulated uptake was determined in BAT, brain (hypothalamus) epiWAT, BAT and ingWAT, heart and gastrocnemius muscle. 15-week-old db/db mice were bilaterally injected with vehicle or chABC into the ARC. q) Body weights, r) adiposity, s) glucose tolerance and t) HOMA-IR were assessed. Results are mean±SEM; significance determined using b, c, e, f, o, q, r) two-way ANOVA with repeated measures, d, g, k, l, m, p, s, t) t test. Scale bar, 100 μm.

FIG. 5: Neurofibrosis in obesity promotes ARC insulin resistance. a-c) C57BL/6J mice were fed a chow or HFHS diet for 12 weeks and bilaterally injected with vehicle or chABC into the ARC. 2 or 8 days post ARC injection, mice were administered vehicle or insulin and brains processed for immunohistochemistry monitoring for b,c) insulin-induced p-AKT expression. d-h) C57BL/6J mice were fed a HFHS diet for 12 weeks and bilaterally injected with vehicle or chABC into the ARC. 4 days post intraARC injection, mice were administered with insulin-FITC and FITC expressing f) area, g) intensity and h) insulin-FITC induced AKT phosphorylation within the ARC was quantified. i, j) Insulin-FITC was incubated with CSPG-ECM components and insulin binding was assessed in vitro. Results are mean±SEM; significance determined using c) two-way ANOVA with Tukeys multiple comparisons test and f-j) one-way ANOVA with Tukeys multiple comparisons test. Scale bar, 100 μm.

FIG. 6: The effects of ARC neurofibrosis on whole-body metabolic dysfunction are driven by impaired AgRP IR signaling. a) Schematic of AAV-gIR construct to conditionally target the mouse IR. b) 12-week HFHS fed AgRP-Cas9 mice received bilateral intraARC injections of AAV-gScrambled or AAV-gTR. ARC targeting was confirmed by analysis of GFP and mCherry immunofluorescence (inserts in b). 1-week later mice received bilateral injections of vehicle or chABC to disassemble neurofibrosis in the ARC and c) body weight, d) adiposity, e) food intake, f) energy expenditure, g) glucose tolerance and h) HOMA-IR was determined. Results are mean±SEM; significance determined using c,d) two-way ANOVA with repeated-measures and e, f, g, h) one-way ANOVA with Tukeys multiple comparisons test. Scale bar, 100 μm.

FIG. 7: Pharmacologically targeting neurofibrosis promotes weight loss and improves glycaemic control in obesity. a) 12-week HFHS fed male mice received daily I.C.V. administration of vehicle or fluorosamine for 10 days. B,c) CSPG-ECM expression in the ARC, d) body weight, e) adiposity, f) energy expenditure, g) food intake, and h) glucose tolerance was assessed. Following 10 days of vehicle or fluorosamine treatment mice were administered insulin and brains processed for immunohistochemistry monitoring for i,j) insulin-induced p-AKT expression. Hyperinsulinemic-euglycemic clamps were performed in conscious unrestrained C57BL/6J mice fed a HFHS diet for 12 weeks and administered with fluorosamine daily for 3 days I.C.V. and k) GIR was assessed. C57BL/6J mice fed a HFHS diet and treated with low dose STZ to phenocopy aspects of late stage T2D received daily I.C.V. administration of vehicle or fluorosamine for 14 days. 1) Daily blood glucose and m) glucose tolerance was assessed. n) 12-week HFHS fed AgRP-Cas9 mice received bilateral injections of AAV-gScrambled or AAV-gIR. 1-week later mice received daily I.C.V. administered vehicle or fluorosamine for 10 days and o) body weights, p) food intake, q) energy expenditure and r) body weights, n) glucose tolerance was assessed. Results are mean±SEM. Significance determined using d, e, k, l, o) two-way ANOVA with repeated-measures and c, f, g, h, j, m, p, q, r) one-way ANOVA with multiple comparisons. Scale bar, 100 μm.

FIG. 8: Intranasal drug administration delivered biotinylated fluorosamine (PZ6005) to the ARC. a) Schematic overview of intranasal administration of biotinylated PZ6005. 7 to 8 weeks-old chow-fed C57BL/6J mice I.N. received vehicle or biotinylated PZ6005 (5 mg/animal/day) for 3 consecutive days. Mice brains and lungs were then extracted for immunohistochemistry to detect the presence of drug, and d-h) the intensity was quantified. Scale bar: b) 500 μm or 100 μm, and f) 200 μm. Results are mean±SEM. Statistical significance was determined using unpaired t test.

FIG. 9: Intranasal administration of fluorosamine (PZ6005) attenuates ARC neurofibrosis in diet-induced obesity. a) Schematic overview of intranasal administration of PZ6005. a) 12-week-HFHS-diet fed C57BL/6J mice received I.N. administration of vehicle or PZ6005 (1 or 5 mg/animal/day) for 14 consecutive days. b) Mice brains were then extracted and processed for WFA immunohistochemistry to determine CSPG-ECM expression within the ARC, and the c,d) area and e,f) the intensity were quantified. Scale bar: 100 μm. Results are mean±SEM. Statistical significance was determined using one-way ANOVA with Tukey's multiple comparison test.

FIG. 10: Pharmacological suppression of ARC neurofibrosis using fluorosamine (PZ6005) induces weight loss in diet-induced obesity. 12-week-HFHS-diet fed C57BL/6J mice were I.N. administered with vehicle or PZ6005 (1 or 5 mg/animal/day) for 14 consecutive days. a,b) Effects on mice body weight was measured each day for 14-day treatment. Results are mean±SEM. Statistical significance was determined using two-way ANOVA with repeated measures and Tukey's multiple comparisons test.

FIG. 11: Pharmacological suppression of ARC neurofibrosis using fluorosamine (PZ6005) reduces adiposity in diet-induced obesity. 12-week-HFHS-diet fed C57BL/6J mice were I. N. delivered with vehicle, PZ6005 (1 mg or 5 mg/animal/day) for 14 consecutive days. a,b) Adipose tissues and liver were extracted and weighed for determination of tissue-specific adiposity, and c,d) fat mass was assessed following 14-day treatment. Results are mean±SEM. Statistical significance was determined using a,b,d) one-way ANOVA with Tukey's multiple comparisons test and c) two-way ANOVA with repeated measures and S̆idák's multiple comparisons test.

FIG. 12: Pharmacological suppression of ARC neurofibrosis using fluorosamine (PZ6005) decreases food intake and increases energy expenditure in diet-induced obesity. 12-week-HFHS-diet fed C57BL/6J mice were daily I.N. administered with vehicle or PZ6005 (1 or 5 mg/animal/day) for 14 days. At Day 8-Day 11 of the treatment, a) 24 hrs food intake, b) cumulative food intake, c,d) oxygen consumption and e-g) energy expenditure were determined. Results are mean±SEM. Statistical significance was determined using a,e) one-way ANOVA with Tukey's multiple comparisons test and c,f) two-way ANOVA with repeated measures and S̆idák's multiple comparisons test.

FIG. 13: Pharmacological attenuation of ARC neurofibrosis using fluorosamine (PZ6005) attenuates ARC insulin resistance in diet-induced obesity. 12-week-HFHS-diet fed C57BL/6J mice were daily I. N. delivered with vehicle or PZ6005 (1 or 5 mg/animal/day) for 14 days. After 14 days of treatment, mice were then I.P. delivered with insulin (5 mg/g). Brain were extracted 15 mins post injection and then processed for immunohistochemistry a) to detect p-AKT signalling induced by insulin. b) ARC pAKT +ve. cells were quantified. Scale bar: 100 μm. Results are mean±SEM. Statistical significance was determined using b) one-way ANOVA with Tukey's multiple comparisons test.

FIG. 14: Central administration of PZ6005 suppresses ARC neurofibrosis in diet-induced obesity. a) Schematic overview of I.C.V. administration of PZ6005 and S.C. injection of liraglutide. 12-week-HFHS-diet fed C57BL/6J mice I.C.V. received vehicle or PZ6005 (100 μg/animal) every other day for the first 14 days of the experiment, while the mice were S.C. administered with vehicle or liraglutide (200 μg/kg/day) for 28 consecutive days. b) Extracted mice brains were then processed to determine ARC CSPG-ECM expression immunostained by WFA, and the c,d) area and e,f) intensity were quantified. Results are mean±SEM. Statistical significance was determined using c,d,e,f) one-way ANOVA with Tukey's multiple comparison test.

FIG. 15: Pharmacological suppression of ARC neurofibrosis using PZ6005 in combination with systemic GLP-1R agonists promotes ARC insulin receptor signalling in diet-induced obesity. 12-week-HFHS-diet fed C57BL/6J mice were I.C.V. delivered with vehicle or PZ6005 (100 μg/animal) every other day for the first 14 days of the experiment, while the mice were S.C. injected with vehicle or liraglutide (200 μg/kg/day) for 28 consecutive days. Following 28 days of the experiment, mice were then I.P. delivered with insulin (5 mg/g of body weight). Brains were extracted 15 mins post injection and processed for immunohistochemistry to determine a) p-AKT signalling induced by insulin. b) ARC pAKT +ve. cells were quantified. Results are mean±SEM. Statistical significance was determined using b) one-way ANOVA with Tukey's multiple comparisons test.

FIG. 16: Pharmacological suppression of ARC neurofibrosis using PZ6005 in combination with systemic GLP-1R agonists improves GLP-1R agonist-induced pERK signalling in diet-induced obesity. 12-week-HFHS-diet fed C57BL/6J mice I.C.V. received vehicle or PZ6005 (100 μg/animal) every other day for first 14 days of the experiment, and were daily S.C. delivered with vehicle or liraglutide (200 μg/kg/day) for 28 days. Following 28 days of the experiment, mice were then I.P. injected with liraglutide (200 μg/kg). Brains were extracted 15 mins after the injection and were processed for immunohistochemistry to detect a) p-ERK signalling induced by GLP-1R agonist. b) Total ARC pERK +ve. cells, c) pERK +ve. cells inside the ARC and b pERK +ve. cells surrounding the ARC were quantified. Results are mean±SEM. Statistical significance was determined using b) one-way ANOVA with Tukey's multiple comparisons test.

FIG. 17: Pharmacological inhibition of ARC neurofibrosis using PZ6005 in combination with systemic GLP-1R agonists induces weight loss in diet-induced obesity. 12-week-HFHS-diet fed C57BL/6J mice were I.C.V. administered with vehicle or PZ6005 (100 μg/animal) every other day for the first 14 days of the experiment, while the mice were S.C. injected with vehicle or liraglutide (200 μg/kg/day) for 28 consecutive days. a,b) Body weight was measured each day for 28-day treatment. Results are mean±SEM.

Statistical significance was determined using a,b) two-way ANOVA with repeated measures and Tukey's multiple comparisons test. @Significance represents Liraglutide (200 mg/kg/day, S.C.)+Vehicle (I.C.V.) treatment group versus Liraglutide (200 μg/kg/day, S.C.)+PZ6005 (100 μg/animal, I.C.V.) treatment group. #Significance represents Vehicle (S.C.)+PZ6005 (100 μg/animal, I.C.V.) treatment group versus Liraglutide (200 μg/kg/day, S.C.)+PZ6005 (100 μg/animal, I.C.V.) treatment groups.

FIG. 18: Pharmacological inhibition of ARC neurofibrosis using PZ6005 in combination with systemic GLP-1R agonists reduces adiposity in diet-induced obesity. 12-week-HFHS-diet fed C57BL/6J mice received I.C.V. administration of vehicle or PZ6005 (100 μg/animal) every other day for the first 14 days of the experiment, while the mice were daily S.C. injected with vehicle or liraglutide (200 μg/kg/day) for 28 days. a) Adipose tissues and liver were extracted and weighed for determination of tissue-specific adiposity. b,c) Mice fat mass was assessed following 28-day treatment. Results are mean±SEM. Statistical significance was determined using a,b,d) one-way ANOVA with Tukey's multiple comparisons test and b two-way ANOVA with repeated measures and Sidak's multiple comparisons test.

FIG. 19: Pharmacological inhibition of ARC neurofibrosis using PZ6005 in combination with systemic GLP-1R decreases food intake and increases energy expenditure in diet-induced obesity. 12-week-HFHS-diet fed C57BL/6J mice were I.C.V. delivered with vehicle or PZ6005 (100 μg/animal) every other day for the first 14 days of the experiment, while the mice were S.C. injected with vehicle or liraglutide (200 μg/kg/day) for 28 consecutive days. At Day 8-Day 11 of the treatment, a) 24 hrs food intake b,c) oxygen consumption, d,e) energy expenditure and g) ambulatory activities were determined. Results are mean±SEM. Statistical significance was determined using a) one-way ANOVA with Tukey's multiple comparisons test and b,d) two-way ANOVA with repeated measures and Sidak's multiple comparisons test.

FIG. 20: Pharmacological inhibition of ARC neurofibrosis using PZ6005 in combination with systemic GLP-1R agonists enhances GLP-1R agonist-improved glycaemic control in diet-induced obesity. 12-week-HFHS-diet fed C57BL/6J mice were I.C.V. given with vehicle or PZ6005 (100 μg/animal) every other day for the first 14 days of the experiment, while the mice were S.C. administered with vehicle or liraglutide (200 μg/kg/day) for 28 consecutive days. a,b) Effects on glucose tolerance was assessed at day 14 following a bolus of glucose (2 mg/g of lean mass, I.P.). Results are mean±SEM. Statistical significance was determined by b) one-way ANOVA with Tukey's multiple comparisons test. In graph b). @Significance indicates Liraglutide (200 μg/kg/day, S.C.)+Vehicle (I.C.V.) treatment group versus Liraglutide (200 μg/kg/day, S.C.)+PZ6005 (100 μg/animal, I.C.V.) treatment group, and #Significance indicates Vehicle (S.C.)+PZ6005 (100 μg/animal, I.C.V.) treatment group versus Liraglutide (200 μg/kg/day, S.C.)+PZ6005 (100 μg/animal, I.C.V.) treatment group.

FIG. 21. Therapeutic inhibition of brain neurofibrosis prolongs rebound effects of GLP-1R agonist-induced weight loss in diet-induced obesity and T2D. 12-week-HFHS-diet fed and aged-matched chow-diet fed C57BL/6J mice were I.C.V. administered with vehicle or PZ6005 (100 μg/animal) every other day, while the mice were S.C. injected with vehicle or liraglutide (200 μg/kg/day) for 14 consecutive days during treatment phase. Mice were then monitored for 48 days in off-treatment phase. a,b,c) Body weight was measured each day for the 65-day experiment and d,e,f) Mice fat and lean mass was assessed following 65-day experiment. H) Adipose tissues and liver were then extracted and weighed for determination of tissue-specific adiposity. Results are means±SEM. Statistical significance was determined using a,b,c) two-way ANOVA with repeated measures and Tukey's multiple comparisons test and d,e,f,g) two-way ANOVA with repeated measures and S̆idák's multiple comparisons test.

FIG. 22. Therapeutic inhibition of brain neurofibrosis enhances energy expenditure in diet-induced obesity and T2D, compared to monotherapy of GLP-1R agonist. 12-week-HFHS-diet fed and aged-matched chow-diet fed C57BL/6J mice were I.C.V. administered with vehicle or PZ6005 (100 μg/animal) every other day, while the mice were S.C. injected with vehicle or liraglutide (200 μg/kg/day) for 14 consecutive days during treatment phase. Mice were then monitored for 48 days in off-treatment phase. a) Effects on oxygen consumption, b) energy expenditure, c) fat & carbohydrates utilizations and d) ambulatory activities were assessed during treatment phase. Results are means±SEM. Statistical significance was determined using a,b,c,d) one-way ANOVA with Tukey's multiple comparisons test and two-way ANOVA with repeated measures and S̆idák's multiple comparisons test.

FIG. 23. Therapeutic inhibition of brain neurofibrosis maintains increased energy expenditure in diet-induced obesity, compared to monotherapy of GLP-1R agonist. 12-week-HFHS-diet fed and aged-matched chow-diet fed C57BL/6J mice were I.C.V. administered with vehicle or PZ6005 (100 μg/animal) every other day, while the mice were S.C. injected with vehicle or liraglutide (200 μg/kg/day) for 14 consecutive days during treatment phase. Mice were then monitored for 48 days in off-treatment phase. a) Effects on oxygen consumption, b) energy expenditure, c) fat & carbohydrates utilizations and d) ambulatory activities were assessed during off-treatment phase. Results are means±SEM. Statistical significance was determined using a,b,c,d) one-way ANOVA with Tukey's multiple comparisons test and two-way ANOVA with repeated measures and S̆idák's multiple comparisons test.

FIG. 24. Therapeutic inhibition of brain neurofibrosis enhances GLP-1R agonists actions upon hypophagia in diet-induced obesity and T2D. 12-week-HFHS-diet fed and aged-matched chow-diet fed C57BL/6J mice were I.C.V. administered with vehicle or PZ6005 (100 μg/animal) every other day, while the mice were S.C. injected with vehicle or liraglutide (200 μg/kg/day) for 14 consecutive days during treatment phase. Mice were then monitored for 48 days in off-treatment phase. a) Effects on 24 hrs food intake were assessed during treatment phase and off-treatment phase. 7 days post treatment phase, overnight fasted mice were given access to food ad libitum. b,c) Mouse behaviour (feeding, drinking, grooming, active, inactive and resting) were assessed every 30 sees for 90 mins, and data were collected into 5 mins time bins and displayed as the percentage of total behaviours. d,e) Transition time from feeding to resting/satiety over the 90 mins period was assessed. In graph d), time point at which feeding behaviour predominantly transitions to resting behaviour is depicted by the dashed line. Results are means±SEM. Statistical significance was determined using a,e) one-way ANOVA with Tukey's multiple comparisons test and b) two-way ANOVA with repeated measures and S̆idák's multiple comparisons test.

FIG. 25. Therapeutic inhibition of brain neurofibrosis prolongs rebound effects of GLP-1R agonists action upon improved glycemic control in diet-induced obesity and T2D. 12-week-HFHS-diet fed and aged-matched chow-diet fed C57BL/6J mice were I.C.V. administered with vehicle or PZ6005 (100 μg/animal) every other day, while the mice were S.C. injected with vehicle or liraglutide (200 μg/kg/day) for 14 consecutive days during treatment phase. Mice were then monitored for 48 days in off-treatment phase. a,b,c) Effects on glucose tolerance was assessed at treatment phase and off-treatment phase following a bolus of glucose (2 mg/g of lean mass, I.P.) and d) insulin tolerance was assessed after weight rebound to 100% of previous body weight following a bolus of insulin (0.8 mU/g of body weight for HFF mice and 0.6 mU/g of body weight for chow fed mice). e) Effects on 24 hrs fasting blood glucose level were determined following 65 days of the experiment. Results are means±SEM. Statistical significance was determined using a,b,c,d,e) one-way ANOVA with Tukey's multiple comparisons test.

### Definitions

Unless defined otherwise, all technical and scientific terms used herein have the same meanings as commonly understood by one of ordinary skill in the art to which this invention belongs.

As used herein, the terms “composition” and “formulation” have been used interchangeably and have the same meaning.

Unless otherwise specified, the indefinite articles “a”, “an” and “the” as used herein, include plural aspects. Thus, for example, reference to “an agent” includes a single agent, as well as two or more agents; reference to “the composition” or “formulation” includes a single composition or formulation, as well as two or more compositions or formulations; and so forth.

As used herein, the term “about” means±10% of the recited value.

Throughout this specification and the claims that follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The term “consisting of” means “consisting only of”, that is, including and limited to the integer or step or group of integers or steps, and excluding any other integer or step or group of integers or steps.

The term “consisting essentially of” means the inclusion of the stated integer or step or group of integers or steps, but other integer or step or group of integers or steps that do not materially alter or contribute to the working of the invention may also be included.

The reference to any prior art in this specification is not, and should not be taken as an acknowledgment or any form of suggestion that that prior art forms part of the common general knowledge.

Other definitions are provided throughout the specification.

### DETAILED DESCRIPTION

The present invention relates to pharmaceutical combinations comprising a 4-epimerase inhibitor and a GLP-1R agonist. Such combinations may be suitable for treating or preventing insulin resistance and associated disorders, such as type-2 diabetes and obesity. The present inventors have identified neurofibrosis in the arcuate nucleus of the hypothalamus (ARC) as a novel disease mechanism underlying central insulin resistance and the development of metabolic disease, and that administration of 4-epimerase inhibitors may reduce or prevent neurofibrosis in the ARC. The present inventors have now shown that therapeutic attenuation of ARC neurofibrosis may enhance GLP-1R agonist activity. For example, the use of a 4-epimerase inhibitor in combination with a GLP-1R agonist may enhance energy and glucose metabolism mediated by the GLP-1R agonist. Thus, a 4-epimerase inhibitor and a GLP-1R agonist may be suitable in combination for treating or preventing insulin resistance and associated disorders, particularly type-2 diabetes and obesity, suppressing appetite and/or promoting weight loss. The terms “type-2 diabetes mellitus”, “type-2 diabetes” and “T2D” are used interchangeable herein and have the same meaning.

Excessive deposition and remodelling of extracellular matrix (ECM) promotes fibrosis and is an established disease mechanism underpinning insulin resistance within muscle, adipose and liver tissue. However, both insulin resistance and fibrosis have been traditionally viewed as peripheral tissue-centric phenomena and the incidence and relevance of the ECM in the brain to the development of metabolic diseases has not previously been explored. A distinct species of ECM has recently been described in the ARC of humans and mice (Alonge, et al., 2020; Mirzadeh, et al., 2019), which is composed of specialised perisynaptic aggregates of hyaluronic acid, chondroitin sulfate proteoglycans (CSPG) and chondroitin sulfate-glycosaminoglycans side chains. The present inventors have identified that the CSPG-ECM within the ARC is a unique multicellular agglomeration concentrated proximally to the median eminence (ME), providing an extracellular juncture between the neural and peripheral endocrine systems. Thus, the CSPG-ECM provides an interface connecting circulating metabolic hormones entering the ARC with metabolically relevant ARC neurons, such as agouti-related peptide neurons (AgRP) and pro-opiomelanocortin (POMC) neurons.

The present inventors have identified that the development of insulin resistance and associated disorders, such as obesity and type-2 diabetes is underscored by CSPG-ECM remodelling at both the component and glycosaminoglycan level, representing a previously unidentified characteristic of insulin resistance and associated disorders, a phenomenon termed “neurofibrosis”. Neurofibrosis within the ARC impedes the penetrance of circulating insulin, which can result in neuronal insulin resistance. Remodelling of CSPG components, including changes to chondroitin sulfate-glycosaminoglycans (CS-GAG) sulfation patterns, may be mediated through elevated CS-0S, CS-4S and CS-2S6S sulfation, which promotes a rigid CSPG-ECM structure that sequesters extracellular diffusion. CS-4S sulfation drives the activity of chondroitin sulfate N-acetylgalactosaminyltransferase-1 (CS-GalNAcT-1), which in turn promotes aggrecan expression, a key CSPG species underlying neurofibrosis in the ARC. The CSPG-ECM remodelling underscoring neurofibrosis specifically occurs around AgRP neurons, which are critical regulators of metabolism and are essential for survival. Impairments in ARC insulin signalling may promote the development of obesity and diabetes through enhanced feeding behaviour, attenuated energy expenditure and defective glucose metabolism.

4-Epimerase (also known as UDP-galactose 4-epimerase) is an enzyme essential for creating the nucleotide sugar substrate UDP-N-acetylgalactosamine required for the assembly and elongation of CS-GAG chains on CSPGs, a core feature of neurofibrosis in the ARC. Thus, the present inventors postulated that 4-epimerase inhibitors may reduce or prevent neurofibrosis in the ARC, representing a novel treatment for insulin resistance and associated disorders. By way of non-limiting example, the present inventors have shown that fluorosamine (1; Ac-4-F-GlcNAc), a fluorinated N-acetyl-D-glucosamine analogue previously identified as inhibiting chondroitin sulfate proteoglycan (CSPG) synthesis (Keough et al., 2016; Stephenson et al., 2019), preferentially attenuates CSPG-ECM in the ARC. This effect may be mediated by the comparatively rapid CSPG-ECM turnover rate seen within the ARC, which enhances the functional efficacy of fluorosamine in the ARC over other brain areas with a slower degradation rate. As there is little CSPG-ECM expression in the amygdala, orbitofrontal cortex and ventral striatum, targeting the brain ECM may limit off-target effects on depression and anxiety, which have undermined prior attempts to pharmacologically target the brain to treat metabolic disease.

The present inventors have also found that attenuation of ARC neurofibrosis (e.g., using a 4-epimerase inhibitor) may enhance GLP-1 receptor (GLP-1R) agonist activity. GLP-1, as an incretin hormone, is predominantly secreted and released from enteroendocrine L-cells in the distal small intestine and colon. GLP-1 acts as a metabolic supporter of insulin, as it promotes the insulin secretory response and inhibits glucagon secretion for maintaining glucose homeostasis. GLP-1 is also responsible for enhancing cell survival and triggering the proliferation and differentiation of 3-cells. The actions of GLP-1R agonists (including co-agonists such as GIP/GLP-1 and glucagon/GIP/GLP-1 agonists) to promote the remission of metabolic diseases such as obesity and type-2 diabetes is dependent upon the action of these compounds within the ARC. The present inventors have found that the development of neurofibrosis in the ARC impedes the penetrance, bioavailability and/or signalling of various GLP-1R agonists and insulins into the brain, and that administering a 4-epimerase inhibitor in combination with a GLP-1R agonist may improve the penetrance, bioavailability and/or signalling of the GLP-1 receptor agonist and insulins. As GLP-1R agonists cross the blood brain barrier through the same mechanism as GLP-1 co-agonists, such as GIP/GLP-1 receptor agonists and triple glucagon/GIP/GLP-1 receptor agonists, it is also highly plausible that 4-epimerase treatment will also enhance or improve the activity (e.g., penetrance, bioavailability and/or signalling) of GLP-1 co-agonists. In some embodiments, the activity of the GLP-1R agonist (or co-agonist) is synergistically enhanced when combined with a 4-epimerase inhibitor.

The 4-epimerase inhibitors used in the combinations disclosed herein may be any known 4-epimerase inhibitors, including but not limited to a fluorinated N-acetyl-glucosamine derivative, such as fluorosamine (1). Stephenson et al., 2019 previously identified that the most effective fluorinated N-acetyl-glucosamine derivatives in reducing chondroitin sulfate GAG stubs attached to the core protein had substitutions on only the anomeric carbon (C-1), such as a hydroxyl group, 0-acetyl group or O-propionyl group, and at least one fluorine at C-4. Thus, as used herein, the term fluorinated N-acetyl-glucosamine “derivative” may refers in particular to an N-acetyl-glucosamine (preferably N-acetyl-D-glocosamine) core structure substituted at C-1 with a hydroxyl or —OC(O)C₁₋₄alkyl (preferably a hydroxyl, 0-acetyl or O-propionyl group) and at C-4 with one or two fluoro groups. Stephenson et al., 2019 also identified that it may be advantageous to include removable acyl protecting groups at 04 and 06.

Thus, in one or more embodiments, a 4-epimerase inhibitor suitable for use in the present invention is a compound of Formula (I):

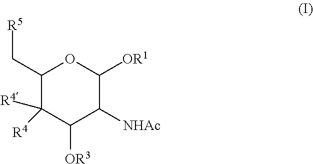

or a pharmaceutically acceptable salt, solvate or hydrate thereof, or a stereoisomer thereof,
wherein:
R¹, R³ and R⁵ are independently selected from H or C(O)C₁₋₄alkyl; and
R⁴ and R⁴′ are independently selected from H and fluoro, wherein at least one of R⁴ and R⁴′ is fluoro.

As used herein, the term “alkyl” refers to a monovalent (“alkyl”) and divalent (“alkylene”) straight chain or branched chain saturated aliphatic groups. The alkyl group may have from 1 to 4 carbon atoms, denoted C₁₋₄alkyl, or it may have from 1 to 3 carbon atoms, denoted C₁₋₃alkyl, or it may have from 1 to 2 carbon atoms, denoted C₁₋₂alkyl. Examples of suitable alkyl groups may include, but are not limited to, methyl, ethyl, 1-propyl, isopropyl, 1-butyl, 2-butyl, isobutyl, sec-butyl and tert-butyl.

It will be recognized that the fluorinated N-acetyl-glucosamine derivatives disclosed herein (or other 4-epimerase inhibitors) may possess asymmetric centers and are therefore capable of existing in more than one stereoisomeric form. Thus, the 4-epimerase inhibitors, such as the fluorinated N-acetyl-glucosamine derivatives disclosed herein, may exist as single stereoisomers, racemates, and/or mixtures of enantiomers and/or diastereomers. Thus, unless otherwise specified, any reference to a fluorinated N-acetyl-glucosamine derivative herein includes stereoisomers thereof. As used herein, the term “stereoisomer” refers to any two or more isomers that have the same molecular constitution and differ only in the three dimensional arrangement of their atomic groupings in space. Stereoisomers may be diastereoisomers or enantiomers. In some embodiments, the fluorinated N-acetyl-glucosamine derivatives disclosed herein may be in substantially pure isomeric form at one or more asymmetric centers (e.g., greater than about 90% ee, 95% ee, 97% ee or 99% ee), or a mixture (including racemic mixtures) thereof.

Preferably, the fluorinated N-acetyl-glucosamine derivative is a N-acetyl-D-glucosamine derivative compound of Formula (IA):

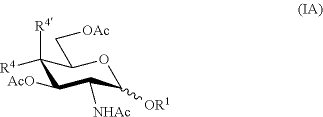

or a pharmaceutically acceptable salt, solvate or hydrate thereof, or a stereoisomer thereof, wherein:
R¹, R³ and R⁵ are independently selected from H or C(O)C₁₋₄alkyl; and
R⁴ and R⁴′ are independently selected from H and fluoro, wherein at least one of R⁴ and R⁴′ is fluoro.

In preferred embodiments of the compounds of Formula (I) and Formula (IA), R¹, R³ and R⁵ are independently selected from H or C(O)C₁₋₃alkyl, more preferably R¹, R³ and R⁵ are independently selected from H or H or C(O)C₁₋₂alkyl.

In preferred embodiments of the compounds Formula (I) and Formula (IA), R¹ is H or C(O)C₁₋₂alkyl, and R³ and R⁵ are both acyl groups.

In preferred embodiments of the compounds of Formula (I) and Formula (IA), R¹, R³ and R⁵ are each acyl groups.

In preferred embodiments of the compounds of Formula (I) and Formula (IA), R⁴ is fluoro and R⁴′ is H, or R⁴ is H and R⁴′ is fluoro, or R⁴ and R⁴′ are both.

In one or more preferred embodiments, the compound of Formula (IA) is selected from:

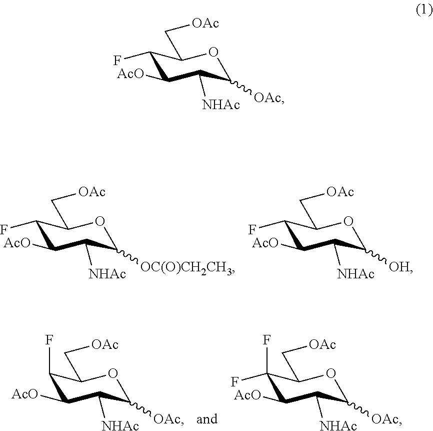

or a pharmaceutically acceptable salt, solvate or hydrate thereof, or a stereoisomer thereof.

In a preferred embodiment, the compound of Formula (IA) is:

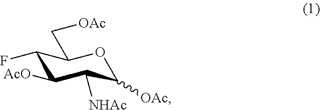

or a pharmaceutically acceptable salt, solvate or hydrate thereof, or a stereoisomer thereof.

Suitable methods for preparing fluorinated N-acetyl-glucosamine derivatives are described by Keough et al., 2016 and Stephenson et al., 2019. Other methods for the preparation of N-acetyl-glucosamine derivatives will be apparent to those skilled in the art.

Other 4-epimerase inhibitors that may be suitable for use in the present invention have also previously been described. For example, the xyloside Ac-bXyl-TEG (2) described by Stephenson et al., 2019, and the aminooxy- and hydrazide-functionalized uridine derivatives described by Winans and Bertozzi, 2002.

Thus, in an embodiment, the 4-epimerase inhibitor is:

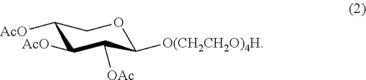

or a pharmaceutically acceptable salt, solvate or hydrate thereof, or a stereoisomer thereof.

In another embodiment, the 4-epimerase inhibitor is a compound of Formula (II), Formula (III) or Formula (IV):

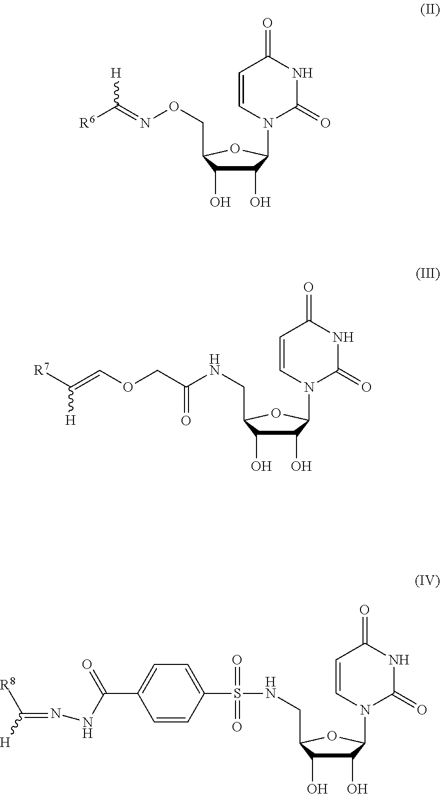

or a pharmaceutically acceptable salt, solvate or hydrate thereof, or a stereoisomer thereof,
wherein:
R⁶ is selected from:

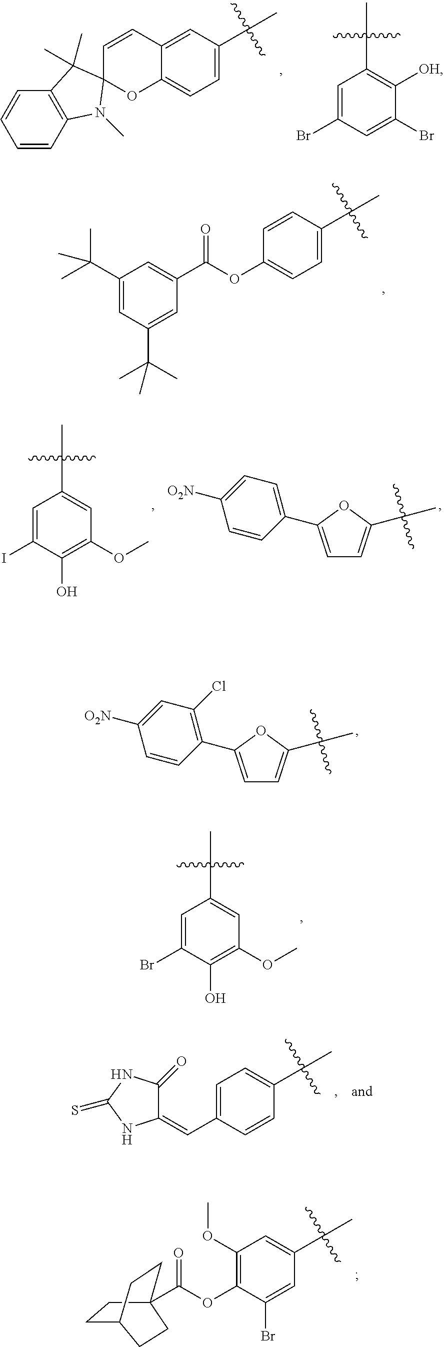

R⁷ is selected from:

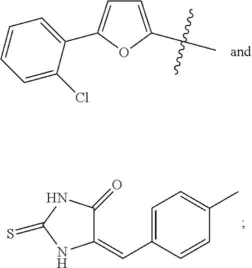

R⁵ is selected from:

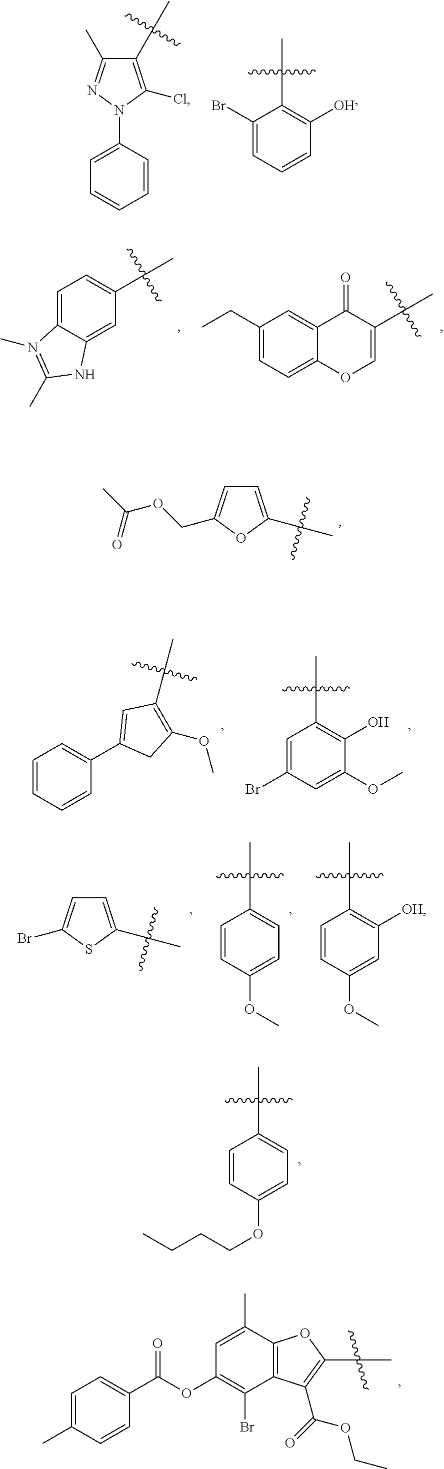

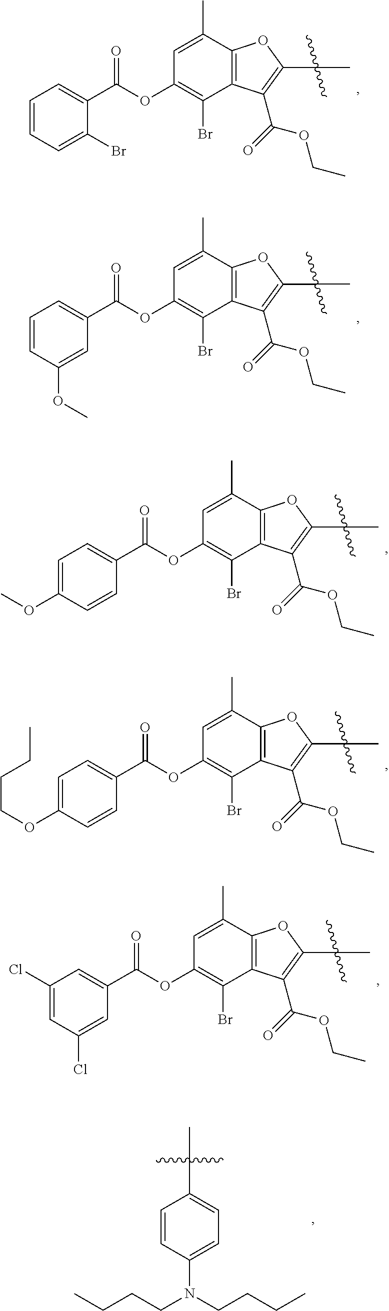

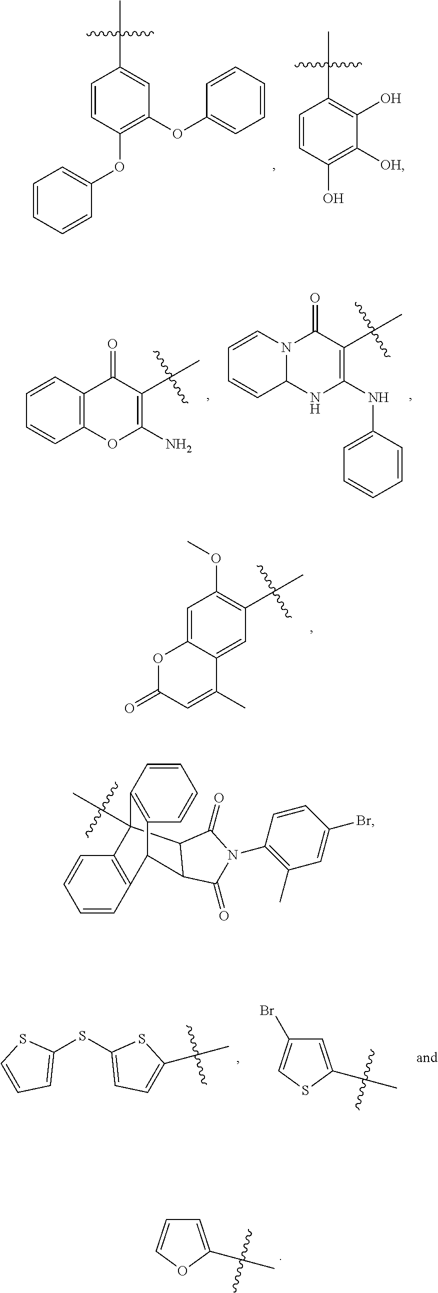

The combinations of the present invention further comprise a GLP-1 receptor (GLP-1R) agonist (also referred to as a “GLP-1 agonist”). The GLP-1R agonists used in the combinations of the present invention may be any GLP-1R agonist, including GLP-1 or an analogue thereof. It is to be understood that such analogues are limited to analogues that maintain the ability (at least in part) to agonise GLP-1R. A number of suitable GLP-1 analogues are currently available on the market, including liraglutide (Saxenda™ Voctoza™), dulaglutide (Trulicity™), exenatide (Bydureon™, Byetta™), semaglutide (Ozempic™) and lixisenatide (Lyxumia™. Adlyxin™), among others. In a particular embodiment, the GLP-1R agonist is liraglutide. However, it is to be understood that the GLP-1R agonist may include both chemical compounds (e.g., small molecules) and biological compounds (e.g., peptides, including recombinant fusion proteins and synthetic peptides), which may or may not be commercially available. Other non-limiting examples of GLP-1R agonists can be found in WO 98/08873, WO 2016/097108, WO 2021/187886, WO 2021/160127, the entire contents of each of which are incorporated herein by cross-reference. Other suitable GLP-1R agonists (including GLP-1 and analogues thereof) and processes for their preparation will be known to those skilled in the art.

The term GLP-1R agonist is intended to encompass GLP-1R co-agonists (also referred to as “GLP-1 co-agonists”), including dual agonists, triple agonists, and the like. Examples of suitable co-agonists may include, but are not limited to, GIP/GLP-1 co-agonists/glucacon/GLP-1 co-agonists, and glucagon/GIP/GLP-1 co-agonists. In an embodiment, the GLP-1 agonist is a GIP/GLP-1 co-agonist. An example of a commercially available GIP/GLP-1 co-agonist that may be suitable for use in the present invention is tirzepatide (Mounjaro™). Other non-limiting examples of GIP/GLP-1 co-agonists are described in WO 2011/119657, WO 2016/111971, WO 2013/164483, WO 2014/192284 and WO 2015/086729, the entire contents of each of which are incorporated herein by cross-reference. In another embodiment, the GLP-1 agonist is a glucacon/GLP-1 co-agonist. Non-limiting examples of GLP-1/glucacon co-agonists are described in WO 2008/101017 and WO 2017/100107, the entire contents of each of which are incorporated herein by cross-reference. In another embodiment, the GLP-1 agonist is a glucagon/GIP/GLP-1 co-agonist. An example of a glucagon/GIP/GLP-1 co-agonist currently in Phase I clinical trials is LY3437943 (Coskun et al., 2022). Other GLP-1 co-agonists are also contemplated by the present invention.

It will be understood that the present invention is not intended to be limited to the particular 4-epimerase inhibitors and/or GLP-1R agonists described herein. In view of the mechanisms underlying the present invention as discovered by the present inventors, it is contemplated that any compound that inhibits 4-epimerase may be suitable for use in the combinations of the present invention. Likewise, it is contemplated that any agonists of the GLP-1 receptor may also be suitable for use in the combinations of the present invention. Preferably, the 4-epimerase inhibitors and GLP-1R agonists are pharmaceutically acceptable compounds. The ability of a compound to inhibit 4-epimerase may be readily determined by one skilled in the art, for example using Western blots for stub chondroitin-4-sulfate attached to the core protein as described by Keough et al., 2016 and Stephenson et al., 2019 or the coupled-enzyme system with a spectrophotometric readout as described by Winans and Bertozzi, 2002. The ability of a compound to agonise GLP-1R may be readily determined by one skilled in the art, for example using GLP-1 and GIP receptor binding assays or receptor internalisation assays.

It is to be understood that, in accordance with the present invention, the 4-epimerase inhibitors (including fluorosamine and other fluorinated N-acetyl-glucosamine derivatives as disclosed herein) and/or GLP-1R agonists may be provided as pharmaceutically salts, hydrates or solvates. The term “pharmaceutically acceptable salts” includes pharmaceutically acceptable solvates and hydrates, and pharmaceutically acceptable addition salts of the 4-epimerase inhibitors and/or GLP-1R agonists, as appropriate. The term “solvate” includes a molecular complex comprising a 4-epimerase inhibitor, GLP-1R agonist. and one or more pharmaceutically acceptable solvent molecules, for example, ethanol. The term “hydrate” is employed when the solvent is water. It is also contemplated that the combinations of the present invention may be suitable for use in the treatment or prevention of insulin resistance and associated disorders in animals. Thus, term “pharmaceutically acceptable salts” is also intended to include veterinarilly acceptable solvates and hydrates, and veterinarilly acceptable addition salts of the 4-epimerase inhibitors, including fluorinated N-acetyl-glucosamine derivatives as disclosed herein, and/or GLP-1R agonists.

In some embodiments, pharmaceutically acceptable salts may include acid addition salts and salts of quaternary amines. A pharmaceutically acceptable salt involves the inclusion of another molecule such as a chloride ion, an acetate ion, a sulfate ion or other counter ion, in the parent compound (i.e., the 4-epimerase inhibitor or GLP-1R agonist). The counter ion may be any organic or inorganic moiety that stabilizes the charge on the parent compound. Furthermore, a pharmaceutically acceptable salt may have more than one charged atom in its structure. When multiple charged atoms are present in the parent compound, its pharmaceutically acceptable salts will have multiple counter ions and these can be several instances of the same counter ion or different counter ions. Hence, a pharmaceutically acceptable salt can have one or more charged atoms in the parent compound and/or one or more counter ions.

Acid addition salts suitable for use in the present invention may be formed from a 4-epimerase inhibitor and/or GLP-1R agonist, and a pharmaceutically acceptable inorganic or organic acid, including but not limited to hydrochloric, hydrobromic, sulfuric, phosphoric, methanesulfonic, toluenesulphonic, benzenesulphonic, acetic, propionic, ascorbic, citric, malonic, fumaric, maleic, lactic, salicylic, sulfamic, or tartaric acids. The counter ion of quaternary amines include chloride, bromide, iodide, sulfate, phosphate, methansulfonate, citrate, acetate, malonate, fumarate, sulfamate, and tartrate. Also, basic nitrogen-containing groups may be quaternised with such agents as lower alkyl halides, such as methyl, ethyl, propyl, and butyl chlorides, bromides and iodides; dialkyl sulfates like dimethyl and diethyl sulfate; and others. The preparation of the pharmaceutically acceptable salts described above and other typical pharmaceutically acceptable salts is more fully described by Berge et al., “Pharmaceutical Salts,” J. Pharm. Sci., 1977:66:1-19.

In some embodiments, salts of 4-epimerase inhibitors and/or GLP-1R agonists may be prepared from the free form of the compound in a separate synthetic step prior to incorporation into a formulation for administration to a subject in accordance with the present invention. In still other embodiments, salts of 4-epimerase inhibitors and/or GLP-1R agonists may be prepared in situ during preparation of a formulation for administration. For example, the formulations for administration may further comprise an appropriate acid which, upon contact with the free form of the 4-epimerase inhibitor and/or GLP-1R agonist forms a desired pharmaceutical salt in situ for administration.

Furthermore, it will recognised by a person skilled in the art that 4-epimerase inhibitors and/or GLP-1R agonists may be provided in crystalline form, either as the free compound or as a solvate (e.g., a hydrate) and it is intended that both forms are within the scope of the present invention. Methods of solvation are generally known within the art.

The present invention also contemplates the use of pharmaceutically acceptable prodrugs of 4-epimerase inhibitors and/or GLP-1R agonists in the treatment or prevention of insulin resistance and associated disorders. For example, the 4-epimerase and/or GLP-1R agonists inhibitor could be provided in the form of a prodrug, which may, upon administration to a subject, be capable of providing (directly or indirectly) the desired 4-epimerase inhibitor, or an active metabolite or residue thereof. The term “prodrug” is used in its broadest sense and encompasses those derivatives that are converted in vivo to the active agent. Such prodrugs would readily occur to those skilled in the art.

As previously described, the present invention encompasses the use of 4-epimerase inhibitors (e.g., fluorinated N-acetyl-glucosamine derivatives) and/or GLP-1R agonists as the free base form or as a pharmaceutically salt or solvate thereof in the treatment of insulin resistance or an associated disorder (e.g., a metabolic disease). Where specific dosages or concentrations of a 4-epimerase inhibitor and/or GLP-1R agonists are referred to herein, it is to be understood that the specific dosage or concentration refers to the concentration of or equivalent to the free base of the 4-epimerase inhibitor and/or GLP-1R agonists. Accordingly, where a pharmaceutically acceptable salt of a 4-epimerase inhibitor and/or GLP-1R agonist is used, a person skilled in the art would readily understand that the concentrations or dosages in respect of the salt, refers to the equivalent concentration or dosage of the free base form of the 4-epimerase inhibitor.

In accordance with the present invention, 4-epimerase inhibitors and/or GLP-1R agonists, or pharmaceutically acceptable salts thereof, may be administered together with one or more pharmaceutically acceptable carriers, diluents, adjuvants and/or excipients. Where a carrier, diluent, adjuvants and/or excipient is used, they must be “pharmaceutically acceptable” in the sense of being compatible with the other ingredients of the composition and not injurious to the subject. Such pharmaceutically acceptable carriers, diluents, adjuvants or excipients will be apparent to those skilled in the art and may depend on the intended mode of administration. For example, the carriers, diluents, adjuvants or excipients may vary depending on the formulation and/or mode of administration. In some embodiments, the 4-epimerase inhibitors and/or GLP-1R agonists may be provided in sustained-release formulations.

In particular, suitable formulations for GLP-1R agonists, particularly for commercially available GLP-1 analogues such as liraglutide, dulaglutide, exenatide, semaglutide and lixisenatide, will be known to those skilled in the art. Thus, in some embodiments, the combinations of the present invention may comprise a GLP-1 agonist in its commercial formulation.

The combinations disclosed herein may comprise the 4-epimerase inhibitor and the GLP-1R agonist in one or more pharmaceutical compositions. For example, the 4-epimerase inhibitor and the GLP-1R agonist may be co-formulated in the same pharmaceutical composition, or they may each be formulated in separate pharmaceutical compositions. Pharmaceutical compositions comprising a 4-epimerase inhibitor and/or GLP-1R agonist for use in the present invention can be prepared by any method known in the art of pharmacology. In general, such preparatory methods include the steps of bringing the 4-epimerase inhibitor and/or GLP-1R agonist into association with one or more carriers, diluents, adjuvants, excipients or other accessory ingredients and then, if necessary and/or desirable, shaping and/or packaging the product into a desired single- or multi-dose unit. In certain embodiments, unit dosage compositions are those containing a daily dose or unit, daily sub-dose, as herein above described, or an appropriate fraction thereof, of the 4-epimerase inhibitor and/or GLP-1R agonist. In some embodiments, the 4-epimerase inhibitor and GLP-1R agonist may be provided in a single unit dose (i.e., as a pharmaceutical composition), or they may be provided in two or more separate unit doses, which may be intended for simultaneous, separate or sequential administration. As used herein, a “unit dose” is discrete amount of the pharmaceutical composition comprising a predetermined amount of the active ingredient. The amount of the active ingredient (i.e., the 4-epimerase inhibitor or GLP-1R agonist) is generally equal to the dosage of the active ingredient that would be administered to a subject and/or a convenient fraction of such a dosage such as, for example, one-half or one-third of such a dosage.

In a preferred embodiment, the 4-epimerase inhibitors may be formulated for intranasal administration. In some embodiments, the intranasal formulation may be prepared as pharmaceutically acceptable emulsions, microemulsions, solutions, or suspensions. In particular, the 4-epimerase inhibitors may be prepared as aqueous solutions or suspensions. Where the 4-epimerase inhibitor formulations are aqueous solutions or suspensions, the formulations may comprise water is in an amount of greater than 50% by weight of the total composition, preferably greater than about 60% by weight of the total composition, more preferably greater than about 70% by weight of the total composition, even more preferably greater than about 80% by weight of the total composition. In still other embodiments, where the formulations disclosed herein are aqueous solutions or suspensions, water may comprise about 80% to about 99% by weight of the total composition, more preferably from about 85% to about 98% by weight of the total composition.

The intranasal compositions disclosed herein may further comprise a pharmaceutically acceptable co-solvent. Suitable co-solvents may include but are not limited to alcohols, polyvinyl alcohols, propylene glycol, polyethylene glycols and derivatives thereof, glycerol, sorbitol, polysorbates, ethanol, and mixtures thereof. In particular, the co-solvent may be selected from glycerol, propylene glycol and mixtures thereof. In still other embodiments, the co-solvent may comprise from about 1% to about 60% by volume of the total composition, preferably from about 2 to about 50%, more preferably from about 3 to about 40%, even more preferably from about 5 to about 35% by volume of the total composition.

The intranasal formulations described herein may comprise a thickening agent. The use of a thickening agent may provide improved adherence of the formulation to the nasal mucosa without adversely affecting the ease of administration, in particular administration as an intranasal spray. Furthermore, a thickening agent may advantageously improve the trans-nasal absorption of the active agent, increase the residence time of the formulation on the nasal mucosa and/or reduce loss of the formulation via mucociliary clearance of the nasal passages. As such, the use of a thickening agent may advantageously provide enhanced bioavailability and/or sustained release of the desired active agent. Thickening agent suitable for use in the present invention may be any pharmaceutically acceptable, nasal mucosa-tolerant thickening agent known to those skilled in the art. The thickening agent may advantageously contribute to the controlled release of the active ingredient on the mucosal membranes. Suitable thickening agents for use in the invention include methylcellulose, ethylcellulose, hydroxyethylcellulose, hydroxypropyl cellulose, hydroxy propyl methylcellulose, sodium carboxy methylcellulose, polyacrylic acid polymers, poly hydroxyethyl methylacrylate, polyethylene oxide, polyvinyl pyrrolidone, polyvinyl alcohol, tragacanth, sodium alginate, araya gum, guar gum, xanthan gum, lectin, soluble starch, gelatin, pectin and chitosan. The amount of thickening agent required to achieve a suitable balance between adherence of the formulation to the nasal mucosa and the sprayability of the formulation may vary depending on the nature of the thickening agent. The amount of a particular thickening agent required to achieve this balance can be determined by a person skilled in the art. For example, the thickening agent may comprise about 0.1% to about 2%, about 0.25% to about 1.5%, or about 0.5% to about 1% by weight of the total composition.

In some embodiments, intranasal formulations suitable for use in the present invention may comprise one or more of a pH modifying agent, sensory agent, antioxidant, surfactant, adhesive, stabilizer, osmolarity adjusting agent, preservative, permeation enhancer, chelating agent, sweetening agent, flavoring agent, taste masking agent, colorant. Some agents or components of the intranasal formulation may have more than one function. For example, where ethanol is used as a sensory agent in the formulations disclosed herein, it may further function as a penetration enhancer and/or a co-solvent.

Suitable additives and amounts thereof for use in intranasal formulations will be apparent to those skilled in the art. By way of example, suitable sensory agents may include a C₂ to C₄ alcohol (such as ethanol or isopropanol) menthols, terpenes, thymol, camphor, capsicum, phenol, carveol, menthol glucuronide, eucalyptus oil, benzyl alcohol, salicyl alcohol, clove bud oil, mint, spearmint, peppermint, eucalyptus, lavender, citrus, lemon, lime, hexylresorcinol, ketals, diols, and mixtures thereof. Examples of suitable preservatives may include benzalkonium chloride, methylparaben, ethylparaben, propylparaben, butylparaben, benzyl alcohol, sodium benzoate, phenylethyl alcohol, and benzethonium.

The GLP-1R agonist may be co-formulated with the 4-epimerase inhibitor (e.g., for intranasal administration), or it may be formulated in a separate dosage form. Suitable formulations for GLP-1R agonists will be known those skilled in the art. In particular, commercially available GLP-1R agonists may be formulated in accordance with the product information and/or manufacturer's specifications, typically in a separate dosage form to the 4-epimerase inhibitor. In some embodiments, the GLP-1 agonist is formulated for parenteral administration, for example, by subcutaneous injection, intravenous injection, intraperitoneally, intramuscular injection, intrasternal injection or infusion. Injectable preparations (i.e., sterile injectable aqueous or oleaginous suspensions) may be formulated according to the known art using, for example suitable dispersing, wetting and/or suspending agents.

General considerations in the formulation and/or manufacture of pharmaceutical compositions can be found, for example, in Remington's Pharmaceutical Sciences, Sixteenth Edition, E. W. Martin (Mack Publishing Co., Easton, Pa., 1980), and Remington: The Science and Practice of Pharmacy, 21st Edition (Lippincott Williams & Wilkins, 2005).

### Therapeutic Use

The present inventors have found that 4-epimerase inhibitors may reduce or prevent neurofibrosis in the ARC, thereby treating or preventing insulin resistance and associated disorders. In addition, the present inventors have found that by reducing or preventing ARC neurofibrosis, 4-epimerase inhibitors may enhance (e.g., synergistically enhance) the effects of GLP-1R agonists, which are known to be suitable for the treatment or prevention of insulin resistance and associated disorders. Thus, combinations of a 4-epimerase inhibitor and a GLP-1R agonist may be particularly suitable for use in treating or preventing insulin resistance and associated disorders, as well as any other indications for which GLP-1R agonists are known to be suitable, such as suppressing appetite and/or promoting weight loss. In some embodiments, administering a 4-epimerase inhibitor in combination with a GLP-1R agonist may prolong the effects of the GLP-1R agonist. For example, the present inventors have demonstrated that the beneficial effects of a GLP-1 analogue (e.g., liraglutide) on both type-2 diabetes and weight loss may be prolonged when co-administered with a 4-epimerase inhibitor (e.g., fluorosamine).

In the context of the present invention disorders associated with insulin resistance may include disorders caused, at least in part, by insulin resistance (e.g., type-2 diabetes) as well as disorders that themselves cause, at least in part, or exacerbate insulin resistance (e.g., obesity). Such disorders may include, but are not limited to prediabetes, type-2 diabetes mellitus, obesity, metabolic syndrome, hypertension, dyslipidemia, atherosclerosis, non-alcoholic fatty liver disease (NAFLD), polycystic ovary syndrome (PCOS), coagulopathy and obstructive sleep apnoea.

Increased appetite and/or weight may be associated with insulin resistance or an associated disorder, or they may associated with insulin dysregulation (e.g., as a precursor to or risk factor for insulin resistance or an associated disorder). Thus, the combinations disclosed herein may be particularly suitable for suppressing appetite and/or promoting weight loss associated with insulin dysregulation.

In an embodiment, the combinations disclosed herein may be particularly suitable for use in the treatment or prevention of obesity, suppressing appetite and/or promoting weight loss.

In accordance with the present invention, a 4-epimerase inhibitor and a GLP-1R agonist may be administered in combination to a subject in need of treatment (e.g., for insulin resistance or an associated disorder, to suppress appetite or promotion weight loss) or they may be administered in a prophylactic sense. In particular, it is clear that the methods of the invention may be used prophylactically as well as for the alleviation of symptoms of insulin resistance or an associated disorder. References herein to “treatment” or the like may therefore include such prophylactic treatment, as well as therapeutic treatment of acute conditions or symptoms. Accordingly, in one or more embodiments, the present invention provides a 4-epimerase inhibitor in combination with a GLP-1R agonist for use in the therapeutic treatment of insulin resistance or an associated disorder, to suppress appetite and/or to promote weight loss. In other embodiments, the present invention provides a 4-epimerase inhibitor in combination with a GLP-1R agonist for use in the prophylactic treatment of insulin resistance or an associated disorder, to suppress appetite and/or to promote weight loss.

Accordingly, the present invention relates to a method of treating or preventing insulin resistance or an associated disorder in a subject comprising administering to the subject an effective amount of a 4-epimerase inhibitor in combination with a GLP-1R agonist.

The present invention also relates to a method of suppressing appetite and/or promoting weight loss in a subject comprising administering to the subject an effective amount of a 4-epimerase inhibitor in combination with a GLP-1R agonist.

The present invention also relates to use of a 4-epimerase inhibitor and a GLP-1R agonist in the manufacture of one or more medicaments for treating or preventing insulin resistance or an associated disorder, suppressing appetite and/or promoting weight loss in a subject.

The present invention also relates to use of a 4-epimerase inhibitor in the manufacture of a medicament for treating or preventing insulin resistance or an associated disorder, supressing appetite and/or promoting weight loss in a subject in combination with a GLP-1R agonist.

The present invention further relates to a combination comprising a 4-epimerase inhibitor and a GLP-1R agonist for use in treating or preventing insulin resistance or an associated disorder, suppressing appetite and/or promoting weight loss in a subject.

The terms “treat”, “treating” or “treatment” with regard to a condition (including a disease or disorder as described herein) refers to alleviating or abrogating the cause and/or the effects of the condition. As used herein, the terms “treat”, “treatment” and “treating” refer to the reduction or amelioration of the progression, severity and/or duration of the condition, or the amelioration of one or more symptoms (e.g., one or more discernible symptoms) of the condition (i.e., “managing” without “curing” the condition), resulting from the administration of one or more therapies (e.g., one or more therapeutic agents such as a 4-epimerase inhibitor as disclosed herein). In specific embodiments, the terms “treat”, “treatment” and “treating” refer to the amelioration of at least one measurable physical parameter of a condition described herein, such as insulin resistance or an associated disorder. In other embodiments the terms “treat”, “treatment” and “treating” refer to the inhibition of the progression of a condition described herein, either physically by, e.g., stabilization of a discernible symptom or physiologically by, e.g., stabilization of a physical parameter, or both.

The terms “preventing” and “prophylaxis” as used herein refer to administering a medicament beforehand to avert or forestall the appearance of one or more symptoms of a condition. The person of ordinary skill in the medical art recognizes that the term “prevent” is not an absolute term. In the medical art, it is understood to refer to the prophylactic administration of a drug to substantially diminish the likelihood or seriousness of a condition, or symptom of the condition and this is the sense intended in this disclosure. As used in a standard text in the field, the Physician's Desk Reference, the terms “prevent”, “preventing” and “prevention” with regard to a condition refer to averting the cause, effects, symptoms or progression of a condition prior to the condition fully manifesting itself.

In some embodiments, the subject in need of treatment or prevention of insulin resistance or an associated disorder is a mammal. The term “mammal” as used herein includes humans, primates, livestock animals (e.g., horses, cattle, sheep, pigs, donkeys), laboratory test animals (e.g., mice, rats, guinea pigs), companion animals (e.g., dogs, cats) and captive wild animals (e.g., kangaroos, deer, foxes). Preferably, the mammal is a human.

In accordance with the present invention, the combinations disclosed herein are to be administered to the subject in need thereof in a treatment effective amount. In some embodiments, a treatment effective amount is a therapeutically effective amount or a prophylactically effective amount. The term “therapeutically effective amount” as used herein means an amount of a 4-epimerase inhibitor and of a GLP-1R agonist sufficient to treat or alleviate the symptoms associated with insulin resistance or an associated disorder, to supress appetite and/or to promote weight loss. The therapeutically effective amount of the compound to be administered will be governed by such considerations, and is either, an incremental maximum tolerated dose, or the minimum amount, necessary to ameliorate, cure, or treat the condition or one or more of its symptoms. The term “prophylactically effective amount” refers to an amount effective in preventing or substantially lessening the chances of acquiring a disease or disorder or in reducing the severity of the disease or disorder before it is acquired or reducing the severity of one or more of its symptoms before the symptoms develop. Generally, prophylactic measures may be divided between primary prophylaxis (to prevent the development of a disease or symptom) and secondary prophylaxis (whereby the disease or symptom has already developed and the patient is protected against worsening of this process).

As used herein, the term “effective amount” relates to an amount of a 4-epimerase inhibitor and a GLP-1R agonist which, when administered in combination according to a desired dosing regimen, provides the desired therapeutic activity. For example, an effective amount of a 4-epimerase inhibitor and a GLP-1R agonist may be an amount or amounts that, when administered in combination, are sufficient to inhibit, slow, interrupt, halt, prevent or arrest insulin resistance. Suitable effective amounts may depend on the age, gender, weight and general health of the patient and can be determined by the attending physician. Suitable dosages for each active agent (i.e., the 4-epimerase inhibitor and the GLP-1R agonist) or the combination thereof may lie within the range of about 0.1 ng per kg of body weight to 100 g per kg of body weight per dosage. The dosage may be in the range of 1 μg to 10 g per kg of body weight per dosage, such as is in the range of 1 mg to 1000 mg per kg of body weight per dosage. In one embodiment, the dosage may be in the range of 1 mg to 500 mg per kg of body weight per dosage. In another embodiment, the dosage may be in the range of 1 mg to 250 mg per kg of body weight per dosage. In yet another embodiment, the dosage may be in the range of 1 mg to 200 mg per kg of body weight per dosage, such as up to 50 mg per kg body weight per dosage.

The terms “administer”, “administering” or “administration” in reference to a combination or component thereof disclosed herein means introducing the active agents (i.e., the 4-epimerase inhibitor and GLP-1R agonist) into the system of the subject in need of treatment. Administration of the combinations provided herein is to be understood to include simultaneous, separate or sequential administration of the active agents.

As used herein, the terms “in combination” or “co-administration” can be used interchangeably to refer to the use of more than one therapy (e.g., one or more prophylactic and/or therapeutic agents). The use of the terms does not restrict the order in which therapies (e.g., prophylactic and/or therapeutic agents) are administered to a person in need thereof. In the context of the present invention, co-administration encompasses administration of the 4-epimerase inhibitor and the GLP-1R agonist in an essentially simultaneous manner, such as in a single pharmaceutical composition, for example, having a fixed ratio of first and second amounts, or as discrete dosage forms. In addition, such co-administration also encompasses use of each compound in a sequential manner in either order. When co-administration involves the separate or sequential administration of a first amount of a 4-epimerase inhibitor and a second amount of a GLP-1R agonist, they are administered sufficiently close in time to have the desired therapeutic effect. For example, the period of time between each administration which can result in the desired therapeutic effect, can range from minutes to hours and can be determined taking into account the properties of each compound such as potency, solubility, bioavailability, plasma half-life, and kinetic profile.

In certain embodiments, an effective amount of a 4-epimerase inhibitor, a GLP-1R agonist, or a combination thereof for administration one or more times a day to a 70 kg adult human may comprise about 0.0001 mg to about 4000 mg, about 0.0001 mg to about 3000 mg, about 0.0001 mg to about 200 mg, about 0.001 mg to about 1500 mg, about 0.01 mg to about 1000 mg, about 0.1 mg to about 1000 mg, about 1 mg to about 1000 mg, about 1 mg to about 100 mg, about 10 mg to about 1000 mg, or about 100 mg to about 1000 mg, of the 4-epimerase inhibitor per unit dosage form. In certain embodiments, formulations of the 4-epimerase inhibitor may be at dosage levels sufficient to deliver from about 0.001 mg/kg to about 100 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, and from about 1 mg/kg to about 25 mg/kg, of subject body weight per day, one or more times a day, to obtain the desired therapeutic effect. In certain embodiments, an effective amount of a 4-epimerase inhibitor for intranasal administration to a 70 kg adult human may comprise about 0.0001 mg to about 4000 mg, about 0.0001 mg to about 3000 mg, about 0.0001 mg to about 200 mg, about 0.001 mg to about 1500 mg, about 0.01 mg to about 1000 mg, about 0.1 mg to about 1000 mg, about 1 mg to about 1000 mg, about 1 mg to about 100 mg, about 10 mg to about 1000 mg, or about 100 mg to about 1000 mg, of an extract or compound per unit dosage form.

In some embodiments, a single dose may be sufficient to treat or prevent insulin resistance and associated disorders, supress appetite and/or promote weight loss, which may be delivered in one or more aliquots (e.g., one or more sprays of an intranasal formulation per nostril) to achieve the desired dose. In other embodiments, multiple doses may be required to treat or prevent insulin resistance and associated disorders. The frequency of dosing may occur at intervals of minutes, hours, days, weeks, months or years or continuously over any one of these periods. The administered amount may be an amount sufficient to treat or alleviate the symptoms associated with the insulin resistance or associated disorder, to supress appetite and/or promote weight loss.

In particular, the dose, dosing frequency and/or duration of treatment with the 4-epimerase inhibitor may any suitable amount and/or timing sufficient to supress ARC neurofibrosis to an extent required to enhance activity of the GLP-TR agonist. For example, the 4-epimerase inhibitor may be administered twice daily, once daily, every second day, weekly, monthly or at greater intervals as required to sufficiently suppress ARC neurofibrosis. In an embodiment, the 4-epimerase inhibitor is administered once every second day. The GLP-1 agonist may be administering according the same or a different dose, dosing frequency and/or duration of treatment as the 4-epimerase inhibitor.

Suitable dosages are known for approved agents and can be adjusted by a person skilled in the art according to the condition of the subject, the type of condition(s) being treated and the amount of a compound or composition being used. In particular, an effective dose and frequency for a commercially available GLP-1R agonist may be in accordance with the product information for the specific product. By way of non-limiting example, an effective amount of liraglutide may be 0.6 mg, 1.2 mg, 1.8 mg, 2.4 mg or 3 mg administered daily; an effective amount of dulaglutide may be 1.5 mg administered weekly; an effective amount of exenatide may be 5 μg or 10 μg administered twice daily, or 2 mg administered weekly; an effective amount of semaglutide may be 0.25 mg, 0.5 mg or 1 mg, 3 mg, 7 mg or 14 mg administered weekly; and an effective amount of lixisenatide may be 10 g or 20 g administered daily. A skilled person will be able to determine other suitable doses and frequencies depending in the particular GLP-1R agonist. Administration of GLP-1R agonists is typically by injection, particularly subcutaneous injection (e.g., liraglutide, dulaglutide, exenatide, semaglutide, lixisenatide) or orally, particularly in tablet form (e.g., semaglutide). However, any suitable mode of administration is contemplated by the present invention.

The intranasal formulations disclosed herein may be administered to a person in need thereof by any suitable intranasal delivery method. Suitable methods for intranasal administration would be well known to a person skilled in the art. The intranasal formulations disclosed herein can be administered as a spray or drop. Accordingly, suitable commercial packages containing the intranasal formulation can be in any spray container known in the art. In one or more embodiments, the formulations disclosed herein may be administered via a spray device or container. Spray devices may be single unit dose systems or multiple dose systems, for example comprising a bottle, a pump and/or an actuator. Such spray devices are available commercially, for example, from Nemera, Aptar, Bespak and Becton-Dickinson. In still other embodiments, the formulations disclosed herein may be administered via an electrostatic spray device, such as described in U.S. Pat. No. 5,655,517. Other suitable means for administering formulations intranasally in accordance with the invention include via a dropper, a syringe, a squeeze bottle, and any other means known in the art for applying liquids to the nasal mucosa in an accurate and repeatable fashion.

The spray devices used to administer the intranasal formulation can range from single-use metered-dose spray devices, multiple-use metered dose nasal spray devices and are not limited to spraying the solutions into each naris but can be administered as a gentle liquid stream from a plunger, syringe or the like or as drops from a unit-dose or multi-dose squeeze bottle, or other means known in the art for applying liquids to the nasal mucosa in an accurate fashion.

In one or more embodiments, a spray device suitable for use with the invention may typically deliver a volume of liquid in a single spray actuation in the range of from 0.01 to 0.15 mL. A typical dosing regimen for a nasal spray product may be in the range of one spray into a single nostril (naris) to two sprays into each nostril (naris). Repeat dosing of the same nostril (naris) may also be undertaken. It is recognised that the dosing schedule, including a repeat dosing schedule, may be modified to obtain a desired pharmacokinetic profile. Further, the dosing schedule may be modified to achieve a rapid reduction in severity, preferably cessation, of symptoms of insulin resistance or an associated disorder. In some cases, incremental increases in repeat dosing may be required to achieve a reduction in severity or cessation of symptoms the viral infection. For example, it may be necessary to increase each repeat dose by 25%, 50%, 75%, 100%, 150% or 200% in order to achieve a reduction in severity or cessation of symptoms of insulin resistance or an associated disorder.

It is to be understood that the active agents in the combination (i.e., the 4-epimerase inhibitor and GLP-1R agonist) may be administered at a dose and/or frequency that is more or less than the effective amount for each active agent alone. In particular, where the combination provides a synergistic effect, a lower dose and/or frequency may be equally or more effective than if one of the component active agents are administered alone. In certain embodiments, the 4-epimerase inhibitor and the GLP-1R agonist are each administered in an effective amount (i.e., each in an amount that would be therapeutically effective if administered alone). In other embodiments, the 4-epimerase inhibitor and the GLP-1R agonist are each administered in an amount that alone does not provide a therapeutic effect (a sub-therapeutic dose). In yet other embodiments, the 4-epimerase inhibitor can be administered in an effective amount, while the GLP-1R agonist is administered in a sub-therapeutic dose. In still other embodiments, the 4-epimerase inhibitor can be administered in a sub-therapeutic dose, while the GLP-1R agonist is administered in an effective amount.

The amount of 4-epimerase inhibitor and/or GLP-1R agonist administered per dose or the total volume of combination and components thereof administered will depend on such factors as the nature and severity of the symptoms, the age, weight, and general health of the patient, as well as the mode of administration. It is recognised that relative amounts of excipients, solvents, diluents, salts, thickening agents, sensory agents, buffers, and/or any additional ingredients in a pharmaceutical composition as disclosed herein may also depending upon the identity, size, and/or condition of the subject treated, as well as the mode of administration. For example, in some embodiments, the dosage of 4-epimerase inhibitor and/or GLP-1R agonist required to achieve a therapeutically equivalent effect may be greater for dosage form compared to another. The terms “therapeutic equivalence” or “therapeutically equivalent” as used herein refer to different compositions comprising the same active agent that produce the same clinical effect and safety profile and/or are pharmaceutical equivalents to one another.

Formulations comprising the 4-epimerase inhibitor, GLP-1R agonist or both may be administered in a single dose or a series of doses. Suitable dosage amounts and dosing regimens can be determined by the attending physician and may depend on the particular condition being treated, the severity of the condition as well as the general age, health and weight of the subject. It will be appreciated that dose ranges as described herein provide guidance for the administration of provided pharmaceutical compositions to an adult. The amount to be administered can be determined by a medical practitioner or person skilled in the art.

In certain embodiments, it is envisaged that combination as disclosed herein may be administered to a subject in need thereof as a substitute or replacement for other traditional medication for the treatment of insulin resistance or an associated disorder, to suppress appetite and/or to promote weight loss. In other embodiments, it is envisaged that a combination as disclosed herein may be administered to a subject in need thereof as a supplement or adjunct to traditional medication. In still other embodiments, it is envisaged that a combination as disclosed herein may be administered to a subject in need thereof in the absence of adjunct therapy. Replacing traditional medication for the treatment of insulin resistance or an associated disorder, to suppress appetite and/or to promote weight loss with a combination as disclosed herein may be advantageous, particularly where the traditional medication is associated with one or more adverse effects.

In other embodiments, a combination as disclosed herein may be administered to a subject in need thereof, together with one or more additional therapeutic agents for a discrete period of time, to address specific symptoms of insulin resistance or an associated disorder, to supress appetite and/or promote weight loss. In still other embodiments, the subject in need thereof may be treated with a combination as disclosed herein and one or more additional therapeutic agents (administered sequentially or in combination) for the duration of the treatment period. In one or more embodiments where a combination as disclosed herein is administered together with an additional therapeutic agent (e.g., a third or subsequent therapeutic agent), the additional therapeutic agent may be any therapeutic agent that provides a desired treatment outcome. In particular, the additional therapeutic agent may be selected from known therapeutic agents for the treatment or prevention of insulin resistance or an associated disorders, including one or more symptoms thereof, appetite suppression and/or weight loss. Such therapeutic agent will be known to those skilled in art. By way of non-limiting example, known therapeutic agents for the treatment of obesity or type-2 diabetes, which may be suitable for use in further combination with the combinations of the present invention.

Where a combination of the present invention is administered in further combination with an additional therapeutic agent, the additional agent may be administered in any “effective amount” which provides the desired therapeutic activity, as described above. Suitable dosage amounts and dosing regimens of the additional therapeutic agent can be determined by the attending physician and may depend on the particular condition being treated, the severity of the condition as well as the general age, health and weight of the subject. It will be appreciated that, unless otherwise specified, dose ranges as described herein provide guidance for the administration of provided pharmaceutical compositions to an adult. The amount to be administered to can be determined by a medical practitioner or person skilled in the art.

The 4-epimerase inhibitor, GLP-1R agonist, and formulations thereof may be contained in a kit. The kit may include, for example, the 4-epimerase inhibitor and the GLP-1R agonist, each packaged or formulated individually, or packaged or formulated in combination. Thus, the 4-epimerase inhibitor may be present in a first container, and the GLP-1R agonist in a second container. The container or containers are placed within a package, and the package can optionally include administration or dosage instructions. In an embodiment, the kits disclosed herein may comprise the 4-epimerase inhibitor in a form suitable for intranasal administration and the GLP-1R agonist in a form suitable for injection (e.g., subcutaneous injection), such as a pre-filled pen. The kits may optionally comprise instructions describing a method of using the pharmaceutical combinations in one or more of the methods described herein (e.g., for preventing or treating a metabolic disease). The kit may optionally comprise one or more pharmaceutically acceptable carriers, diluents, adjuvants and/or excipients. The pharmaceutical combination comprising the 4-epimerase inhibitor and the GLP-1R agonist contained in the kit may be optionally combined in the same pharmaceutical composition.

Those skilled in the art will be aware that the invention described herein is subject to variations and modifications other than those specifically described. It is to be understood that the invention described herein includes all such variations and modifications. The invention also includes all such steps, features, methods, compositions and compounds referred to or indicated in this specification, individually or collectively, and any and all combinations of any two or more of said steps or features.

Certain embodiments of the invention will now be described with reference to the following examples which are intended for the purpose of illustration only and are not intended to limit the scope of the generality hereinbefore described.

### EXAMPLES

### Abbreviations

+ve: positive
2-DG: [¹⁴C]2-deoxyglucose
ITT: 3^{rd} ventricle
ARC: arcuate nucleus of the hypothalamus
AUC: area under curve
BAT: brown adipose tissue
chABC: Chondroitinase ABC
CPSG-ECM: chondroitin sulfate proteoglycan extracellular matrix
DAPI: 4′,6-diamidino-2-phenylindole
ddH2O: double-distilled water
EGP: endogenous glucose production
epiWAT: epididymal white adipose tissue
FITC: Fluorescein isothiocyanate
GIR: glucose infusion rate
GLP-1R: glucagon-like peptide 1 receptor
HABP: hyaluronic acid binding protein
HFHS: high-fat high-sugar diet
HOMA-IR: Homeostatic Model Assessment for Insulin Resistance
I.C.V.: intracerebroventricular
I.N.: intranasal
I.P.: intraperitoneally
epiWAT: epididymal white adipose tissue
ingWAT: inguinal white adipose tissue
IR: insulin receptor
NPY: neuropeptide-Y
NZO: New Zealand Obese mouse
p-AKT: AKT Ser-473 phosphorylation
PBS: phosphate buffered saline
PF: par-fed
POMC: pro-opiomelanocortin
PV: parvalbumin
RER: Respiratory exchange ratio
RSG: Retrosplenial cortex
S.C.: subcutaneous
UCP1: uncoupling protein 1
VMH: ventromedial hypothalamus
WFA: Wisteria floribunda agglutinin

### General Procedures

### General Procedure A. Animals

Mice were maintained on a 12 h light-dark cycle in a temperature-controlled high-barrier facility with free access to food and water per NHMRC Australian Code of Practice for the Care and Use of Animals. C57BL/6J and Balb/C mice were sourced from the Animal Resources Centre, Australian, whereas Agrp-IRES-Cre (Strain #:012899), db/db (strain #: 000697), Npy-GFP (Strain #:006417), Pomc-GFP (Strain #:009593), LSL-Cas9 (Strain #:028551), NZO (Strain #:002105) mice were sourced from Jackson Laboratories, USA. To generate Agrp-IRES-Cre; LSL-Cas9-GFP (AgRP-Cas9) mice, hemizygous Agrp-IRES-Cre mice were bred with homozygous LSL-Cas9-GFP mice, Male Sprague-Dawley rats (ARC, Canning Vale, Australia) were housed individually with nesting/enrichment material at a room temperature of 23±2° C., room humidity 40%-70%, on a reverse 12 h light/dark cycle (lights off at 9:00 a.m.). Animals were fed a standard chow (Barastoc, Ridley AgriProducts, Australia) or a high-fat high sugar diet (Mice: 43% and 20% of total energy from fat and carbohydrate respectively, SF04-001; Specialty Feeds, Australia. Rats 30% fats of total energy SF17-204, Specialty Feeds, Australia). To induce late-stage type-2 diabetes in mice, male C57Bl/6J mice were fed a HFHS diet for 4 weeks before being receiving a total of 6 injections of streptozotocin (STZ, 40 mg/kg, i.p. Sigma, in 50 mM sodium citrate buffer pH 4.5) over the following 2 weeks. Blood glucose levels were monitored and mice exhibiting stable blood glucose levels of >15 mM were used for downstream experiments. Experiments were approved by The University of Melbourne Animal Ethics Committee (10323, 10324, 10352, 10385, 10427, 21712, 22282, 22404).

### General Procedure B. Genotyping

DNA extracted from tail biopsies using Tissue Extract-PCR Buffers (MDX004, Meridian Bioscience, OH) and DNA was amplified by PCR using MyTaq™ HS Red Mix (BIO-25048, Meridian Bioscience, OH) with the following primers to detect Cre (forward: 5′ GCG GTC TGG CAG TAA AAA CTA TC ′3 (SEQ ID NO: 1), reverse 5′ GTG AAA CAG CAT TGC TGT CAC TT ′3 (SEQ ID NO: 2)), LSL-Cas9 (wt forward: 5′ AAG GGA GCT GCA GTG GAG TA ′3 (SEQ ID NO: 3), wt reverse: 5′ CAG GAC AAC GCC CAC ACA ′3 (SEQ ID NO: 4), tmt forward: 5′ TCC CCA TCA AGC TGA TCC ′3 (SEQ ID NO: 5), mt reverse: 5′ CTT CTT CTT TGG GGC CAT CT ′3 (SEQ ID NO 6)), Npy-GFP (common forward: 5′ TAT GTG GAC GGG GCA GAA GAT CCA GG ′3 (SEQ ID NO: 7), wt reverse: 5′ CCC AGC TCA CAT ATT TAT CTA GAG ′3 (SEQ ID NO: 8), mt reverse: 5′ GGT GCG GTT GCC GTA CTG GA ′3 (SEQ ID NO: 9)), Pomc-GFP (forward 5′ AAG TTC ATC TGC ACC ACC G ′3 (SEQ ID NO: 10), reverse 5′ TGC TCA GGT AGT GGT TGT CG ′3 (SEQ ID NO: 11)) alleles. The following primers were used to monitor the CRISPR mediated deletion of the mouse InsR (ΔInsr^{CRISPR}): forward 5′ GAG ATG GTC CAC CTG AAG GA ′3 (SEQ ID NO: 12), reverse 5′ GTG AAG GTC TTG GCA GAA GC ′3 (SEQ ID NO: 13).

### General Procedure C. Immunohistochemistry

For immunohistochemistry on brain, mice were anaesthetised and perfused transcardially with heparinised saline [10,000 units/L porcine heparin] followed by 10% neutral buffered formalin. Brains were post-fixed for 16 h and kept for three days at 4° C. in 30% sucrose in PBS to cryoprotect the tissue, before freezing on dry ice. 30 μm sections (120 mm apart) were cut in the coronal plane throughout the entire rostral-caudal extent of the hypothalamus. Sections were stored in cryoprotectant (30% ethylene glycol, 20% glycerol in PBS) at −20° C. for long term storage. For the detection of HABP and versican only, sections were subjected to heat-induced epitope retrieval using citrate acid buffer (10 mM Sodium citrate, 0.05% Tween 20, pH 6.0) at 95° C. for 20 min.

For detection of aggrecan, GFP, HABP, parvalbumin, mCherry, versican, tenascin-C, HAPLN1, neurocan, phosphacan, brevican, WFA and WFA-FITC sections were incubated at room temperature for 1 h in blocking buffer (0.3% Triton X-100, 5% normal goat serum, Gibco, ThermoFisher, MA, 0.02% sodium azide) and then overnight at 4° C. in 1% blocking buffer containing either rabbit anti-aggrecan (1:1000, AB1031, Millipore, MA), chicken anti-GFP (1:2000; ab13970, Abcam, Cambridge, UK), Biotinylated HABP (1:100, 385911, Burlington, MA), sheep anti-parvalbumin (1:1000), in house), rabbit anti-dsRed (1:2000, 600-401-379, Rockland, PA), rabbit anti-versican (1:1000, AB1033, Millipore, MA), tenascin-C (1:500, M1-B4, Developmental Studies Hybridoma Bank, Iowa), HAPLN1 (1:500, 9/30/8-A-4, Developmental Studies Hybridoma Bank, Iowa), neurocan (1:300, 1F6-S, Developmental Studies Hybridoma Bank, Iowa), phophacan (1:300, 3F8, Developmental Studies Hybridoma Bank, Iowa), brevican (1:500, 610895, BD Transduction Laboratories), biotinylated WFA (1:2000, L1516; Sigma-Aldrich, MO), WFA-FITC (1:2000, FL-1351-2, Vector Laboratories, CA), rabbit anti PGP9.5 (1:1000, 14730-1-AP, Proteintech, IL), guinea pig anti AgRP (1:500, AS506, Antibodies Australia, Melbourne, AUS). After washing with PBS-T (0.3% Triton X-100 in PBS, +0.02% sodium azide), sections were incubated with goat anti-chicken Alexa Fluor 488 (ab150169, Abeam, Cambridge, UK), goat anti-rabbit Alexa Fluor-488, 595, 647 (ab150077, ab150080, ab150083, Abcam, Cambridge, UK). donkey anti-sheep Alexa Fluor 594 (ab150180, Abcam, Cambridge, UK), Alexa Fluor 594, 647 Streptavidin (405240, BioLegend, CA)—conjugated secondary antibodies in 5% blocking buffer for 2 h at room temperature. Sections were mounted with Mowiol 4-88 mounting media and visualised with an Olympus BX61 microscope. Images were captured with an Olympus BX61 camera, acquired using Olympus cellSens Dimension software v2.1 and processed using ImageJ software (NIH, MA). Images for cell internalisation were captured using a Zeiss LSM880 Airyscan Fast confocal microscope, acquired using Zeiss ZEN software v2.1 and processed using ImageJ software (NIH, MA). Brightness and contrast in the colour merged images have been adjusted to aid in the analysis of co-incidence.

For ingWAT immunohistochemistry, ingWAT was immediately dissected and fixed in buffered formalin solution on a rocking platform for 48 h. at room temperature Tissues were embedded in paraffin and 5 μm sections 100 μm apart were prepared. For hematoxylin and eosin (H&E) histology sections were incubated in hematoxylin for 3 minutes followed by 30 seconds in eosin. For detection of UCP-1 sections were subjected to antigen retrieval in citrate acid buffer (10 mM Sodium citrate, 0.05% Tween 20, pH 6.0) at 95° C. for 20 min. Sections were incubated at room temperature for 1 h in 5% blocking buffer and then overnight at 4° C. in rabbit anti-UCP-1 (1:1000; ab10983, Abeam, Cambridge, UK), in 1% blocking buffer. Following washing in PBS-T, sections were incubated with goat anti-rabbit Alexa Fluor 488 (ab150077, Abeam, Cambridge, UK) secondary antibody in 5% blocking buffer for 2 h at room temperature. Sections were incubated in DAPI (20 ng/ml in PBS) for 10 min then mounted with Mowiol 4-88 mounting media and visualised with an Olympus BX61 microscope. Images were captured with an Olympus BX61 camera, acquired using Olympus cellSens Dimension software v2.1 and processed using ImageJ software (NIH, MA). Brightness and contrast in the colour merged images have been adjusted to aid in the analysis of co-incidence.

General Procedure D. Functional p-AKT Immunohistochemistry

Mice were injected intraperitoneally with vehicle (PBS) or insulin (3 mU/g, i.p., Actrapid, Nova Nordisk, Denmark) and mice were transcardially perfused (as described above) 15 min with 10% neutral buffered formalin. The brains were post-fixed for 16 h on a rocking platform at RT and then kept for two days in 30% sucrose in PBS to cryoprotect the tissue, before freezing on dry ice. 30 μm sections were cut in the coronal plane throughout the entire rostral-caudal extent of the hypothalamus. Sections were pre-treated for 10 mins in 0.3% glycine, washed in PBS-T and incubated for 10 min in 0.03% SDS. Sections were then blocked in 5% blocking buffer for 1 h at RT and incubated for 48 h with rabbit anti-p-AKT (Ser-473) (1:300; #4060, Cell Signaling Technology, Beverly, MA) in 1% blocking buffer. Sections were then incubated in 5% blocking buffer containing either goat anti-rabbit Alexa Fluor 647 (ab150083, Abcam, Cambridge, UK) or biotinylated goat anti-rabbit (BA-1000, Vector Laboratories, CA, no sodium azide in blocking buffer). Florescence sections were mounted with Mowiol 4-88 mounting media and visualised using Olympus BX61 microscope. Images were captured with an Olympus BX61 camera, acquired using Olympus cellSens Dimension software v2.1 and processed using ImageJ software (NIH, MA). For chromogenic detection, p-AKT signal was amplified using VECTASTAIN® ABC-HRP Kit (1; 500, PK-4000, Vector Laboratories, CA) and visualised using 0.1% H₂O₂ DAB solution (3,30-diaminobenzidine, ICN980681, Thermo Fisher, MA) Peroxidase Substrate Kits (Vector Laboratories, UK). p-STAT3 and p-AKT immunopositive cells were visualised with a Leica DM2000 LED bright field microscope using a Leica DMC6200 camera and Leica Application Suite X software.

### General Procedure E. CSPG-ECM Immunofluorescent Analysis

The ARC CSPG-ECM was stereologically assessed throughout the entire rostro-caudal ARC. The ARC was divided into three regions, including the rostral ARC (−1.22/−1.58 mm anterior-posterior), medial ARC (−1.58/−1.94 mm anterior-posterior) and caudal ARC (−1.94/−2.18 mm anterior-posterior). CSPG-ECM was quantified in the VMH and RSG cortex (−1.58/−1.94 mm anterior-posterior).

All image quantification was performed in Image J (NIH) software (NIH, MA). Raw images underwent background subtraction using a rolling ball algorithm to minimize background and any potential variance in tissue autofluorescence. To quantify area and intensity of the CSPG-ECM within each brain region (ARC, VMH or RSG cortex) images were thresholded and binarized to create a region of interest (ROI) mask of only the CSPG-ECM. For each brain area, CSPG-ECM ROI area (μm²) and intensity (sum of all pixel intensities within the ROI) was calculated. This process was automated to minimise bias and to account for differences in brain nuclei size across multiple images. Brain nuclei were defined in accordance with the Paxinos and Franklin mouse brain atlas (http://labs.gaidi.ca/mouse-brain-atlas/). The area and intensity of CSPG-ECM within each region was normalised to the respective control.

To determine the co-localisation of ECM components (HA, HAPLN1, tenascin-C, aggrecan, versican, phosphacan, brevican, neurocan) within the CSPG-ECM (WFA-positive staining), 2 masks were generated per image: one for the total CSPG-ECM staining and another for component staining within the ARC. The overall area and intensity were calculated for the total CSPG-ECM structure. The area and intensity for components within the CSPG-ECM was determined by quantifying the expression within the total CSPG-ECM mask only. This allowed for the characterisation of ECM components expressed specifically within the ARC CSPG-ECM. The area and intensity of CSPG-ECM within each region was normalised to the respective control. Inversely, to determine the co-localisation of the WFA-labelled ARC CSPG-ECM within the ARC CSPG-ECM components, 2 masks were generated per image; one for total CSPG-ECM staining and another for component staining within the ARC. The overall area and intensity were calculated for the total component structure. The area and intensity for the CSPG-ECM comprising the components was determined by quantifying the WFA expression within the total component mask only. The area and intensity of CSPG-ECM within each region was normalised to the respective control. This combined approach further characterises the specificity of the components to the CSPG-ECM region.

General Procedure F. Quantification of ARC Neurons within the CSPG-ECM

To determine which metabolically relevant ARC neurons are encased within the CSPG-ECM during the development of metabolic disease brains taken from 0, 4 week and 12 week HFHS fed Npy-GFP (to visualise AgRP/NPY neurons) and Pomc-GFP (to visualise POMC neurons) mice were analysed. ARC sections were stained for GFP and WFA as described in Immunohistochemistry section and analysed using Image J (NIH) software. To determine the number of GFP positive neurons encased within the CSPG-ECM two masks were generated. To define the CSPG-ECM structure in the ARC images were thresholded and binarized to create a CSPG-ECM mask. To identify individual GFP positive neurons images were thresholded and binarized to create a GFP mask. To define individual GFP neurons, the GFP masks were segmented using a watershed separation algorithm. The total number of GFP positive cells were counted within the whole ARC area and within the CSPG-ECM mask. This quantified the percent of GFP cells encompassed by the CSPG-ECM in the ARC.

To determine the intensity of the CSPG-ECM that specifically surrounds individual GFP cells in the ARC, GFP images were thresholded and binarized. An ROI of 1.29 μm (average size of ECM surrounding cortical neurons) was generated around each GFP cell using dilate, distance map and Voronoi processes in ImageJ software. This generated a mask capable of specifically analysing CSPG-ECM bordering individual GFP cells. Using this mask, CSPG-ECM staining intensity surrounding GFP cells present within the ARC CSPG-ECM was determined.

### General Procedure G. Behavioural Satiety Sequence

Mice were fasted overnight and housed individually in transparent cages with ad libitum access to water. Two hours after the beginning of the light cycle (9 am) pre-weighed food was presented to the mice and mice were undisturbed and discreetly observed for 90 minutes. Momentary behaviour was scored every 30 seconds over a 90-minute observation. Behaviour at each 30 second interval was recorded according to the following classifications: feeding (animal at hopper trying to obtain food, chewing, or gnawing), drinking (animal licking at the water spout), grooming (animal scratching, biting or licking any part of its anatomy), resting (animal curled up, resting head with eyes closed), active (animal showing activity, including locomotion, sniffing, rearing), or inactive (animal immobile when aware, or signs of sickness behaviour). Data was collated into 5-minute bins, and several variables were assessed including; the average percentage of time the mice spent engaging in each recorded behaviour (% of total behaviour), food intake, the transition from eating to resting and the time to satiety (the time when the frequency of eating behaviour intersects with the frequency of resting behaviour).

### General Procedure H. Hyperinsulinemic Euglycaemic Clamps in Conscious Freely Behaving Mice

For hyperinsulinemic-euglycemic clamps, mice were anesthetised under isoflurane and the right jugular vein was catheterised for infusions, as described previously by Dodd et al., 2018. Catheters were attached to an implant button (BMSW25, RWD Life Sciences, Shenzhen, China). Implant buttons were capped allowing for group mousing of mice and catheters were kept patent by flushing daily with 40 μL saline containing 200 units/mL heparin. On the day of the experiment, food was removed at 7:00 A.M. After 3.5 h fasting, a primed (1 min, 1.25 uCi/min) continuous infusion (0.05 uCi/min) of [3-3H]glucose (NET331A001MC, PerkinElmer, MA) was administered to measure whole-body glucose turnover, as described previously by Dodd et al., 2018. 90 minutes later, mice received a 40 mU/Kg insulin bolus over 10 mins which was followed by continuous insulin infusion (4 mU/kg/min in gelofusine). Euglycemia (~8-10 mM blood glucose) was maintained by a variable infusion of a 30% glucose solution.

Tail blood samples were collected during steady-state conditions (Ra=Rd) and at 80, 90, 100, 110, and 120 min for determination of Rd and Ra, as described above. At 120 mins, a 13 uCi bolus of [¹C]-2-deoxy-D-glucose (NEC495A250UC, PerkinElmer, MA) was injected into the jugular vein, and blood was sampled at 122, 125, 135, 145, and 155 min. At the end of the experiment tissues were extracted for glucose uptake determinations.

### General Procedure I. Pair Feeding

12-week HFHS fed C57BL/6J mice were bilaterally injected with vehicle or chABC into the ARC. 24 h food intake was determined for intraARC chABC treated mice and a cohort of intraARC treated vehicle treated mice were pair-fed, whereby food availability was restricted to the average food consumed by intraARC chABC treated mice.

### General Procedure J. Metabolic Assessment

Metabolic measurements were undertaken in the Melbourne Mouse Metabolic Phenotyping Platform (The University of Melbourne, Australia). Glucose tolerance tests were performed on 6 h fasted conscious mice respectively by injecting D-glucose (2 mg/g of lean body mass and 1 mg/g lean mass for db/db and HFHS+STZ mice) into the peritoneal cavity and measuring glucose in tail blood immediately before and at 15, 30, 45, 60, 90 and 120 min after injection using an Accu-Check glucometer (Roche, Germany). The areas under glucose excursion curves were determined and expressed as mmol/l×min. Fasted (12 h fast) plasma insulin or glucose levels were determined using a Rat/Mouse Insulin ELISA (EZRMI-13K, Merck Millipore, CA) or an Accu-Check glucometer respectively. The HOMA-IR was calculated using the equation [(glucose×insulin)/405]. Adiposity was measured using TD-NMR minispec (Bruker Optics Inc., Billerica, MA).

Mice were acclimated for 24 h and then monitored for 48 h in an environmentally controlled Promethion Metabolic Screening System (Sable Systems International, NV) fitted with indirect open circuit calorimetry, food consumption and activity monitors to measure activity, caloric intake and energy expenditure. Respiratory quotients were calculated as the ratio of CO2 production over O2 consumption respiratory exchange ratio and energy expenditure was calculated using the Weir equation (Kcal h−1=60×(0.003941×VO2+0.001106×VCO2). To account for difference in body mass/composition energy expenditure was analysed and adjusted using ANCOVA using scripts available at the National Mouse Metabolic Phenotyping Centers (MMPC, Nashville, TN, USA) Energy Expenditure Analysis Page (https://www.mmpc.org/shared/regression.aspx).

To provide an index of ingWAT and BAT thermogenesis, infra-red thermography was used to measure temperature changes in the inguinal and inter-scapular regions as described previously (Dodd et al., 2019). The FLIR T1010 thermal imaging camera (FLIR Systems Australia Pty Ltd, VIC, Australia) was mounted onto a tripod and animals were positioned at a standardised distance of 70 cm from the camera. Animals were anaesthetised, shaved in the regions of interest and whole-body images were collected in both the prone and supine positions. Temperatures were analysed using the FLIR ResearchIT Max 4 program (FLIR Systems, OR, United States). The peak temperatures within the ingWAT and BAT was determined.

### General Procedure K. Stereotaxic Surgery

All stereotaxic injections were undertaken under isoflurane anaesthetic using an Ultra Precise Stereotaxic Instrument (963 Kopf, Munich, Germany) or Ultra Precise Rotational Stereotaxic Instrument (69100, RWD Life Sciences, Shenzhen, China) alongside stereotaxic nanoinjectors (788130, KD Scientific, Holliston, MA) with Neurosyringes (Hamilton, NE).

To disassemble the CSPG-ECM within the ARC, mice received bilaterally (unless stated otherwise) administration of 15 mU/side of active chABC (C3667, Sigma, St. Louis, MI, dissolved in 1 M trehalose) or heat-inactivated chABC protein as a vehicle (chABC in 1 M trehalose was heat-inactivated at 85° C. for 45 mins, as described previously by Alonge et al., 2020) in a total volume of 150 nl/side. To pulse the CSPG-ECM within the ARC or RSG, mice received bilateral (unless stated otherwise) administration of biotinylated WFA (0.3 μg/side, in a volume of 150 nl). To disrupt IR in AgRP neurons, 12-week HFHS fed AgRP-Cas9 mice were stereotaxically injected with AAVs expressing U6-driven guide RNA's targeting the InsR gene or a scrambled sequence (5′ GTG TAG TTC GAC CAT TCG TG ′3 (SEQ ID NO: 14)) alongside a CAG driven mCherry FLEX switch. Unless otherwise stated injections were bilaterally into the ARC (coordinates, bregma: anterior-posterior, −1.70 mm; dorsal-ventral, −5.85 mm; lateral, +/−0.18 mm, 200 nl/side) or into the RSG (coordinates, bregma: anterior-posterior, −1.40 mm; dorsal-ventral, −1.80 mm; lateral, +/−0.50 mm, 200 nl/side). WFA-biotin was injected unilaterally into the cc (coordinates, bregma: anterior-posterior, −1.40 mm; dorsal-ventral, −5.80 mm; lateral, +/−0.20 mm, 200 nl/side)

### General Procedure L. Virus Production

To generate AAV-gScrambled (pAAV-U6>mScramble-GTGTAGTTCGACCATTCGTG (SEQ ID NO: 14))-CAG>LL:rev(mCherry):rev(LL):WPRE) and AAV-gIR (pAAV[-U6>mInsr[gRNA-TATCGACTGGTCCCGTATCC (SEQ ID NO: 15)]-U6>mInsr[gRNA-GTCTGTCCAGGCACCGCCAA (SEQ ID NO: 16)]-CAG>LL:rev(mCherry):rev(LL):WPRE) viral vectors, sgRNAs were first designed using online CRISPR tools (http://crispr.mit.edu and http://chopchop.cbu.uib.no/). Potential off-target gRNA binding was assessed in silico using Off-Spotter (https://cm.jefferson.edu/Off-Spotter/) and guides exhibiting ≥3 mismatch with non-specific genomic regions were considered (Anderson, et al., 2015). For AAV-gScrambled a pUp-U6>Scramble gRNA vector was generated using the Gibson assembly of a pDONR P4-P1R backbone and primers 5′ GGGGACAACTTTGTATAGAAAAGTTGGAGGGCCTATTTCCCATGATTC ′3 (SEQ ID NO: 17) and 5′ GGGGACTGCTTTTTTGTACAAACTTGAAAAAAGCACCGACTCGGTGCC ′3 (SEQ ID NO: 18). For AAV-gIR a pUp-U6>mInsr[gRNA-TATCGACTGGTCCCGTATCC (SEQ ID NO: 15)]-U6>mInsr[gRNA-GTCTGTCCAGGCACCGCCAA (SEQ ID NO: 16)] gRNA vector was generated using the Gibson assembly of a AarI digested pUp-U6-gRNA-AarI-Stuffer-AarI backbone and primers 5′ ATATCTTGTGGAAAGGACGAAACACCGTATCGACTGGTCCCGTATCCG ′3 (SEQ ID NO: 19) and 5′ AACTTGCTATTTCTAGCTCTAAAACTTGGCGGTGCCTGGACAGAC ′3 (SEQ ID NO: 20). For both AAV-gScrambled and AAV-gIR the p-Up vectors were cloned alongside pDown-CAG and pTail-LL:rev(mCherry):rev(LL) to generate the final vectors by LR reaction using the Gateway method. AAV vectors were packaged into the AAV-DJ/8 steotype at a titre of >2 10\hat{ }13 GC/ml). All vector cloning and AAV packaging was carried out by VectorBuilder (Chicago, IL).

### General Procedure M. Insulin Extravasation in the ARC

12-week HFHS fed C57BL/6J or aged matched chow fed controls received bilateral injection of vehicle or chABC into the ARC. 3 days post injection (before differences in body weights were seen), mice were fasted for 6 h. To assess insulin extravasation into the ARC mice were administered insulin-FITC (50 μg/animal in a volume of 100 μl, i.v., 13661, Sigma, St. Louis, MI) or FITC (64.3 μmoles/animal in a volume of 100 μl, i.v., F3651, Sigma, St. Louis, MI). Mice were perfused (as described above) 30 mins post injection. To assess insulin extravasation into the ARC irrespective of the BBB, mice were administered insulin-FITC (1 ag/animal in a volume of 2 μl) directly into the lateral ventricles. To do this, mice were anaesthetised and stereotaxically injected (as described above) insulin-FITC at a rate of 200 nl/min into the lateral ventricles (coordinates, bregma: anterior-posterior, −0.20 mm; dorsal-ventral, −2.4 mm; lateral, +0.10 mm). Mice were perfused (as described above) 20 minutes from the start of injection. To assess insulin-FITC brains were post fixed overnight and cryoprotected in 30% sucrose in PBS. To retain spontaneous fluorescence signal, brains and sections we're kept in the dark and we're mounted and imaged immediately after sectioning.

### General Procedure N. Lateral Ventricle Cannulations

Under isoflurane anaesthetic 12-week HFHS fed C57BL/6J or AgRP-Cas9 mice were implanted stereotaxically with guide cannulas into the right lateral ventricle (0.2 mm posterior, 1.0 mm lateral from bregma). Guide cannula was positioned 1.3 mm above the injection site (1 mm ventral to the surface of the skull). AgRP-Cas9 mice were treated with either AAV-gScrambled or AAV-gIR and underwent guide cannula placement 7 days post AAV administration. Mice were administered I.C.V. vehicle (ddH₂O), fluorosamine (100 μg/animal/day or 250 μgl/animal/day) in a volume of 2 μl/animal and all compounds were delivered approximately 1 h before lights off (7 pm).

### General Procedure O. Intranasal Drug Delivery

Conscious mice were restrained by scruffing and inverted parallel to the floor with the chin at ~180-degree angle with the neck. Using a 10 μL tip, a pipettor was loaded with 5 μl of vehicle (ddH₂O) or fluorosamine (1 mg/animal in 20 μl or 5 mg/animal in 20 μl). The tip of the filled pipettor was placed near the left nostril at a 45-degree angle, and the drug was ejected to form a small 5 μl droplet at tip for the mouse to inhale. Immediately after the mouse inhaled the first droplet the remaining solution was ejected to form another small droplet for the mouse to inhale through the same nostril. The mouse was held in this position for 15 seconds before the procedure was repeated in the right nostril. The mouse was returned to the cage for 2 minutes and the process was repeated so that each mouse received four droplets of 5 μl each, delivering a total of 20 μl of solution. All drugs were administered delivered approximately 1 h before lights off (7 pm).

### General Procedure P. CSPG-ECM Tracker Validation and Quantification

To determine CSPG-ECM turnover in the ARC, RSG, or CC, mice received stereotaxic injections of biotinylatated-WFA (WFA-biotin) as described in Stereotaxic Surgery section. At experimental endpoints mice were transcardially perfused and assessment of pulse labelled ARC CSPG-ECM was identified by immunofluorescent detection of WFA-biotin (CSPG-ECM at the time of pulse) and WFA-FITC (total CSPG-ECM) as described in Immunohistochemistry section.

To chase the pulsed WFA-biotin in the ARC, sections were imaged and analysed using Image J (NIH) software. Raw images underwent background subtraction using a rolling ball algorithm to minimize background and tissue autofluorescence. To quantify staining area within the ARC, images were thresholded and binarized to create ROI masks for WFA-biotin and WFA-FITC. For each image, staining ROI area (μm²) and intensity (sum of all pixel intensities within the ROI) was calculated.

To validate the CSPG-ECM tracker technique, 8-week-old C57BL/6J mice were stereotaxically injected unilaterally with WFA (0.3 ag/side, in a volume of 150 nl) to pulse the CSPG-ECM into one side of the ARC and saline injected into the other side. 1 day later mice were transcardially perfused and ARC brain sections were stained and analysed for CSPG-ECM tracker analysis. To determine how faithfully the pulsed WFA-biotin represents the current CSPG-ECM the percentage area to which WFA-biotin (pulse labelled) colocalises with WFA-FITC (total present CSPG-ECM) was quantified.

To validate that chased WFA-biotin signal represents bona fide CSPG-ECM staining WFA (0.3 μg/side, in a volume of 150 nl) was stereotaxically injected bilaterally into the ARC of 8-week-old C57BL/6J mice. 3 days later mice received unilaterally ARC injection of chABC (15 mU/side in a volume of 150 nl) or vehicle to disassemble the WFA-biotin bound CSPG-ECM. To determine the specificity of pulsed WFA-biotin the area and intensity of WFA-biotin staining was quantified and compared in the chABC and vehicle treated sides of the ARC.

To determine CSPG-ECM turnover in lean and obese mice, WFA-biotin (0.3 μg/side, in a volume of 150 nl) was stereotaxically injected bilaterally into the ARC of 12-week HFHS fed C57BL/6J mice or aged matched controls. Brains were extracted either the day after surgery (day 0) or following 1-, 3-, 5-, and 10-weeks post injection. Brain sections were stained for the presence of WFA-biotin and WFA-FITC and the area of WFA-biotin staining was quantified as described above. To determine CSPG-ECM turnover WFA-labelled CSPG-ECM present at the start of the experiment (day 0) was compared to that which remained at weeks 1, 3, 5, and 10. WFA-FITC labelling of the CSPG-ECM was performed at each time point to validate the presence of the ARC CSPG-ECM and ensure changes in WFA-biotin labelling were not due to loss of the CSPG-ECM over time. The same process was used to assess turnover in the RSG and blood vessels of the CC.

### General Procedure Q. ARC CS-GAG and HA Quantitation

Microdissected ARC tissues from male mice fed a HFHS diet for 0, and 12 weeks were incubated in the extraction buffer, containing 8 M urea, 0.5% triton x-100, 5 mM Tris 2-carboxyethylphosphine and cOmplete™ mini ETDA-free protease inhibitor cocktail (Merck) for 30 mins with gentle mixing and then homogenised. Samples were centrifuged for 30 min at 5000 rpm and the supernatant was collected and buffer exchanged using Amicon Ultracell-10k MWCO centrifugal tubes into PBS. Protein concentration of each sample was estimated using a Bradford assay. Twenty n of each protein extract was reduced using 5 mM dithiothreitol for 30 mins at 50° C. and alkylated with 10 mM idoacetamide for an hour at room temperature before blotting onto 0.45 μm PVDF membrane (Millipore, Cat #IPVH20200) and dried overnight. Each sample spot was transferred into a 96 well plate and blocked using 1% (v/v) polyvinylpyrrolidone solution.

The disaccharide analysis procedure was adapted from Moh et al., 2022 with the following modifications. GAG disaccharides were released from the PVDF sample spots using an enzyme mix containing 5 mU chondroitinase ABC (chABC; Sigma, Cat #C3667), 50 ng each of heparinase I/II/III (R&D Systems) in 100 mM ammonium acetate pH 7, 5 mM calcium chloride and incubated at 30° C. overnight. An additional mixture of purified GAG polysaccharides containing 1 μg each of bovine kidney heparan sulfate (Sigma-Aldrich, H7640), 10 μg shark chondroitin sulfate (Sigma-Aldrich, C4382) and 1 μg of Streptococcus equi HA (Sigma-Aldrich, 53747) were digested alongside samples as enzyme reaction control, and as retention time standards. Digested disaccharides were collected and dried under low pressure for labelling using 2-AB (2-aminobenzamide), according to a commercially available protocol (Ludger LT-KAB-VP24-Guide-v2.0). Samples, alongside a standard mix of 8 common HS (Iduron, UK, HS mix) and 8 common CS disaccharides (Iduron, UK, CS mix), were labelled with 2-AB and washed with octanal twice to remove excess labelling agent. Cleaned samples in the aqueous layer were dried and resuspended in 75% acetonitrile with 10 mM ammonium acetate, pH 6.8.

The labelled disaccharides were separated by liquid chromatography using a SeQuant ZIC-HILIC column (3.5 μm, 1 mm×150 mm) at 35° C. using an Agilent 1260 Infinity II with fluorescence detection. The mobile phases solvent A (10 mM NH₄Ac, pH 6.8) and solvent B (90% acetonitrile in 10 mM NH₄Ac pH 6.8) were run at a constant flowrate of 50 l/min in microflow mode with gradient parameters as follows: 0-3 min—100% B. 4 min—90% B, 20 min—88% B. 35 min—70% B, 36-40 min—60% B, 42-50 min—100% B. Fluorescence detection was carried out with excitation and emission wavelengths set at 320 nm and 420 nm, respectively. Peaks were identified using the standard panel and polysaccharide digest control as retention time references and the abundances were quantified manually by peak area.

### General Procedure R. Patch Clamp Electrophysiology

Npy-GFP male mice were placed on a HFHS diet for 12-weeks before being stereotaxically injected with either vehicle (n=4) or chABC (n=4) in the ARC 3 days before electrophysiological characterisation. Mice were anaesthetised with isofluorane prior to brain extraction, and brains were incubated in ice-cold aCSF of the following composition: 127 mM NaCl, 1.2 mM KH₂PO₄, 1.9 mM KCl, 26 mM NaHCO₃, 3 mM D-glucose, 7 mM mannitol, 2.4 mM CaCl₂), 1.3 mM MgCl₂ (saturated with 95% O2 and 5% CO₂, pH 7.4). Coronal sections (250 μm) of the ARC were cut using a vibrotome (Leica VTS1000S, Germany). Slices were heated for 30 min at 34° C. and then allowed to cool to room temperature prior to recording. Slices were placed in a recording chamber and continuously perfused with room temperature aCSF.

Npy-GFP neurons in the ARC were visualised using fluorescence and differential interference contrast optics with infrared video microscopy (AxioCam MRm, Zeiss, Germany) and an upright microscope (BX51WI, Olympus, Germany). For current clamp recordings, patch pipettes (8-11 Mn) were pulled from thin-walled borosilicate glass (Sutter Instruments, BF150-86-10) using a horizontal puller (Sutter Instruments, USA) and filled with intracellular solution containing 140 mM K-gluconate, 10 mM HEPES, 10 mM KCL, 1 mM EGTA, 4 mM Na-ATP, 0.3 mM Na-GTP and 10 mM Biocytin (300 mOsm and pH 7.3, with osmolality and pH adjusted with sucrose and KOH accordingly). In voltage-clamp recordings to examine K+ currents, patch pipettes (3-6 MΩ) were filled with intracellular solution containing 130 mM K-gluconate, 6 mM NaCl, 4 mM NaOH, 11 mM EGTA, 1 mM CaCl₂), 10 mM HEPES, 1 mM MgCl₂, 2 mM Na-ATP, 0.2 mM Na-GTP, 0.1% biocytin (295 mOsm and pH 7.3, with osmolality and pH adjusted with sucrose and KOH accordingly). Cells with a series resistance of >20 MΩ were not included in the analysis. Recordings were made in the presence of tetrodotoxin, where 11 depolarizing pulses applied from −40 to +60 mV for 500 ms in 10 mV increments from a holding potential of −80 mV. A 50 ms prepulse to 0 mV was used to inactivate any residual voltage-dependent Na+ current. Whole-cell recordings were made using a Double IPA integrated patch amplifier controlled with SutterPatch software (Sutter Instruments, USA) with all current clamp data filtered at 5 kHz. Data was analysed using Sutterpatch (Sutter Instruments. USA) and Clampfit 10.7 (Axon Instruments).

### General Procedure S. Immunoblotting

The mediobasal hypothalamus was microdissected and snap frozen in liquid N2. Tissues were mechanically homogenized in 100 ul of ice-cold RIPA lysis buffer (ab156034, Abcam, UK, containing PhosStop Phosphatase Inhibitor, 1 tablet/10 mL; Roche PHOSS-RO) and clarified by centrifugation (13,000×rpm for 20 min at 4° C.). Tissue lysates were resolved by SDS-PAGE and immunoblotted as described previously (PMID: 31509751). Antibodies used are rabbit phospho-IR (Tyr1162, Tyr1163) polyclonal antibody (1:1000, 44-804G, Invitrogen, MA), rabbit monoclonal anti-IR (1:1000, 3025×, Cell Signalling, MA), rabbit-β-actin polyclonal antibody (1:2000, 4967, Cell Signaling Technology), mouse-Gapdh monoclonal antibody (1:5000, 60004-1-Ig, Proteintech, IL), mouse monoclonal anti-tubulin (1:2000, T5168, Sigma).

### General Procedure T. Real-Time PCR

RNA was extracted using TRIzol reagent (Invitrogen, Carlsbad, CA) and total RNA quality and quantity determined using a NanoDrop 3300 (Thermo Scientific, Wilmington, DE, USA). mRNA was reverse-transcribed using a High-Capacity cDNA Reverse Transcription Kit (Applied Biosystems, Foster City, CA) and processed for quantitative real-time PCR using SYBR Green PCR Master Mix (4309155, Applied Biosystems, MA).

The following primers were used for SYBR green expression assays:

Adamst4
(f-GAACGGTGGCAAGTATTGTGAGG (SEQ ID NO: 21),
r-TTCGGTGGTTGTAGGCAGCACA (SEQ ID NO: 22)),
Adamst5
(f-CTGCCTTCAAGGCAAATGTGTGG (SEQ ID NO: 23),
r-CAATGGCGGTAGGCAAACTGCA (SEQ ID NO: 24)),
Il-6
(f-GGTGCCCTGCCAGTATTCTC (SEQ ID NO: 25),
r-GGCTCCCAACACAGGATGA (SEQ ID NO: 26)),
Kcna4
(f-GCAGATTGCTGAATGACACCTCG (SEQ ID NO: 27),
r-GGACAAGCAAAGCATCGAACCAC (SEQ ID NO: 28)),
Kcnb1
(f-GAGGAGTTCGACAACACGTGCT (SEQ ID NO: 29),
r-TGAGTGACAGGGCAATGGTGGA (SEQ ID NO: 30)),
Kcnb2
(f-GCTGGAGAAACCTAACTCGTCC (SEQ ID NO: 31),
r-CTCGTCGTTTTCTTGCAGCTCTG (SEQ ID NO: 32)),
Kcnc3
(f-GAAGAGGTGATTGAAACCAACAGG (SEQ ID NO: 33),
r-TGGGCTCTTGTCTTCTGGAGAC (SEQ ID NO: 34)),
Kcnc4
(f-CCAGCTCGAATCGCCCATTTAC (SEQ ID NO: 35),
r- AGCACCGCATTAGCATCGCCAT (SEQ ID NO: 36)),
Kcnd2
(f-CCTACATGCAGAGCAAGCGGAA (SEQ ID NO: 37),
r-GTGGTTTTCTCCAGGCAGTGAAG (SEQ ID NO: 38)),
Kcnd3
(f-AGAAGAGGAGCAGATGGGCAAG (SEQ ID NO: 39),
r-CTTGATGGTGGAGGTTCGTACAG (SEQ ID NO: 40)),
Kcnj11
(f-TGCGTCACAAGCATCCACTCCT (SEQ ID NO: 41),
r-GGACATTCCTCTGTCACCATGC (SEQ ID NO: 42)),
Kcnj3
(f-CAGTTCGAGGTTGTCGTCATCC (SEQ ID NO: 43),
r- CCCAAAGCACTTCGTCCTCTGT (SEQ ID NO: 44)),
Kcnj6
(f-GGAACTGGAGATTGTGGTCATCC (SEQ ID NO: 45),
r-TCTTCCAGCGTTAGGACAGGTG (SEQ ID NO: 46)),
Kcnj9
(f-TCTCACCTCTCGTCATCAGCCA (SEQ ID NO: 47),
r-GCTTCGAGCTTGGCACGTCATT (SEQ ID NO: 48)),
Kcnma1
(f-CCTGAAGGACTTTCTGCACAAGG (SEQ ID NO: 49),
r-ACTCCACCTGAGTGAAATGCCG (SEQ ID NO: 50)),
Kcnn3
(f-TCCACCGTCATCCTGCTTGGTT (SEQ ID NO: 51),
r-CAGGCTGATGTAGAGGATACGC (SEQ ID NO: 52)),
Kcnq3
(f-AAGCCTACGCTTTCTGGCAGAG (SEQ ID NO: 53),
r-ACAGCTCGGATGGCAGCCTTTA (SEQ ID NO: 54)),
Mmp13
(f-AGCAGTTCCAAAGGCTACAACT (SEQ ID NO: 55),
r-GGATGCTTAGGGTTGGGGTC (SEQ ID NO: 56)),
Mmp14
(f-AGCACTGGGTGTTTGACGAA (SEQ ID NO: 57),
r-CCGGTAGTACTTATTGCCCCG (SEQ ID NO: 58)),
Mmp2
(f-GTCGCCCCTAAAACAGACAA (SEQ ID NO: 59),
r-GGTCTCGATGGTGTTCTGGT (SEQ ID NO: 60)),
Mmp9
(f-GCTGACTACGATAAGGACGGCA (SEQ ID NO: 61),
r-TAGTGGTGCAGGCAGAGTAGGA (SEQ ID NO: 62)),
r18s
(f-CAGCTCCAAGCGTTCCTGG (SEQ ID NO: 63),
r-GGCCTTCAATTACAGTCGTCTTC (SEQ ID NO: 64)),
Tgfβ1
(f-GGATACCAACTATTGCTTCAG (SEQ ID NO: 65),
r-TGTCCAGGCTCCAAATATAG (SEQ ID NO: 66)),
Tgfβ2
(f-CTAATGTTGTTGCCCTCCTACAG (SEQ ID NO: 67),
r-GCACAGAAGTTAGCATTGTACCC (SEQ ID NO: 68)),
Tgfβr1
(f-GGACCATTGTGTTACAAGAAAGC (SEQ ID NO: 69),
r-CATGGCGTAACATTACAGTCTGA (SEQ ID NO: 70)),
Tgfβr2
(f-TCCTAGTGAAGAACGACTTGACC (SEQ ID NO: 71),
r-TACCAGAGCCATGGAGTAGACAT (SEQ ID NO: 72)),
Timp1
(f-TCTTGGTTCCCTGGCGTACTCT (SEQ ID NO: 73),
r-GTGAGTGTCACTCTCCAGTTTGC (SEQ ID NO: 74)),
Timp3
(f-GCTAGAAGTCAACAAATACCAG (SEQ ID NO: 75),
r-TAGTAGCAGGACTTGATCTTG (SEQ ID NO: 76)),
Tnfα
(f-CTGTGAAGGGAATGGGTGTT (SEQ ID NO: 77),
r-GGTCACTGTCCCAGCATCTT (SEQ ID NO: 78)).

Gene expression was normalized to r18s and relative quantification was achieved using the ΔΔCT method. Reactions were performed using a BioRad CFX 384 touch (Bio-Rad, Hercules, CA).

### General Procedure U. CSPG-ECM Binding Assay

To determine the interaction of insulin with CSPG-ECM components in vitro, flat-bottom 96-well plates were first coated with 10 μg/ml poly-L-lysine overnight, followed by a rinsing with water. A purified CSPGs mix containing neurocan, phosphacan, versican and aggrecan (CC117, Merck Millipore, MA), purified aggrecan (A1960, Merck Millipore, MA) or purified chondroitin 4-sulfate (59004, Selleck Chemicals, TX), were coated onto the 96-well plates at a concentration of 10 μg/ml for 4 h at RT, followed by a rinse with water. Insulin-FITC was incubated on plates containing ECM at concentrating ranging from 5 ng/ml-1 mg/ml for 2 h at RT and protected from light. Control wells contained either no ECM, bovine serum albumin (10 μg/ml) or poly-L-lysine alone. Wells were washed 3 times with water and imaged at 495 nm using a SPECTROstar Nano Microplate Reader (BMG Labtech, Germany). To digest CSPG-ECM or to negate CSPG-ECM negative charge. wells were incubated with either chABC (0.5 U/ml) or poly-1-arginine (10 μg/ml, P7762, Merck Millipore, MA) for 1 h at 37° C. after the ECM coating, washed 3 times with water and then incubated with insulin-FITC.

### General Procedure Q. Statistical Analysis

Statistical significance was determined by a one-way or two-way ANOVA with multiple comparisons or repeated-measures, or a one or two-tailed paired Student's t test, or ANCOVA as appropriate, or simple linear regression. p<0.05 was considered significant: *p<0.05, **p<0.01 and ***p<0.001. Statistical details of individual experiments such as exact values of n and exact statistical tests can be found in figures and legends.

### Results

### Example 1. A Unique CSPG-ECM is Present within the ARC

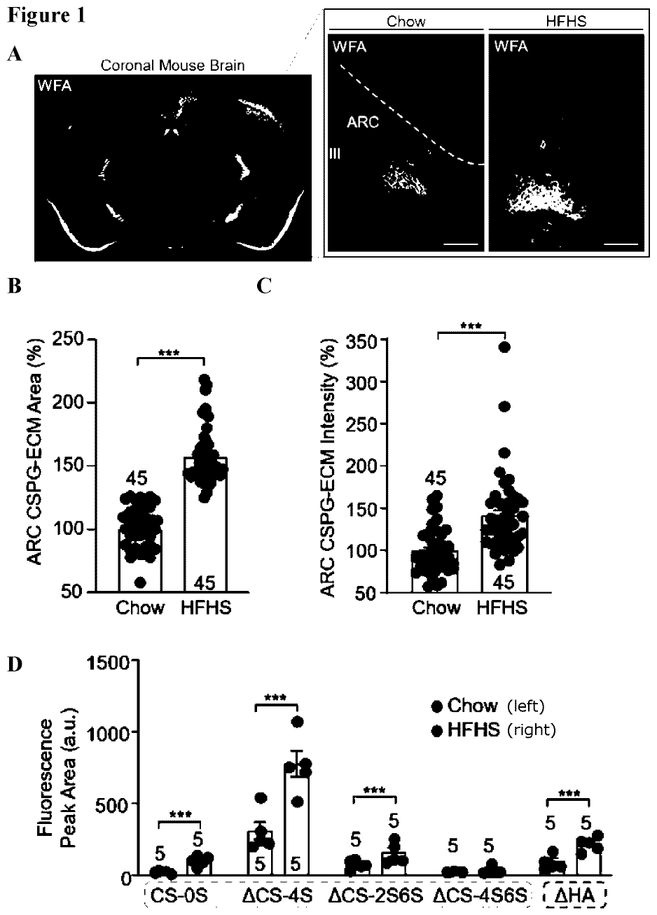

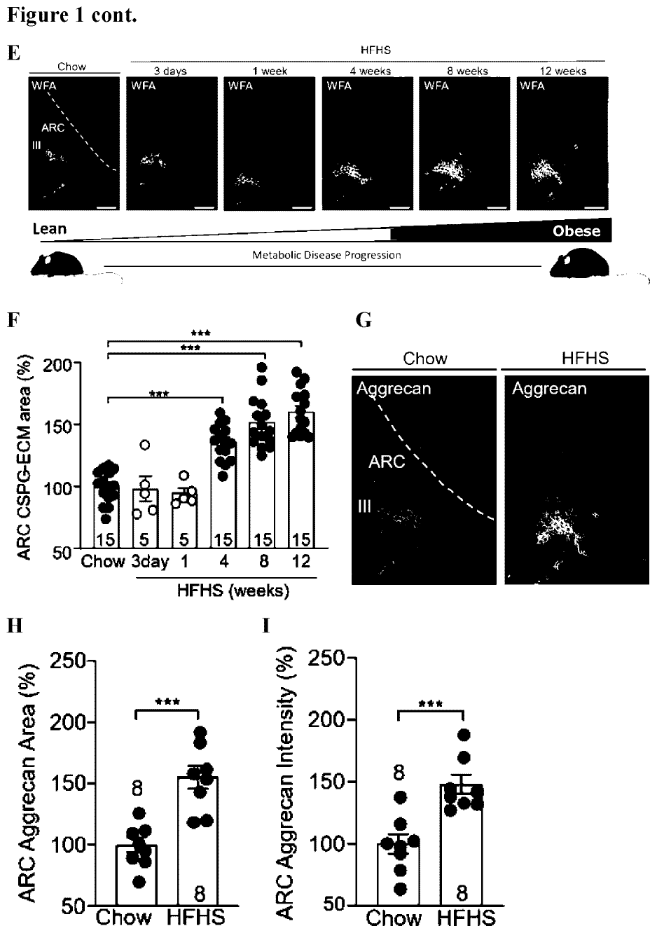

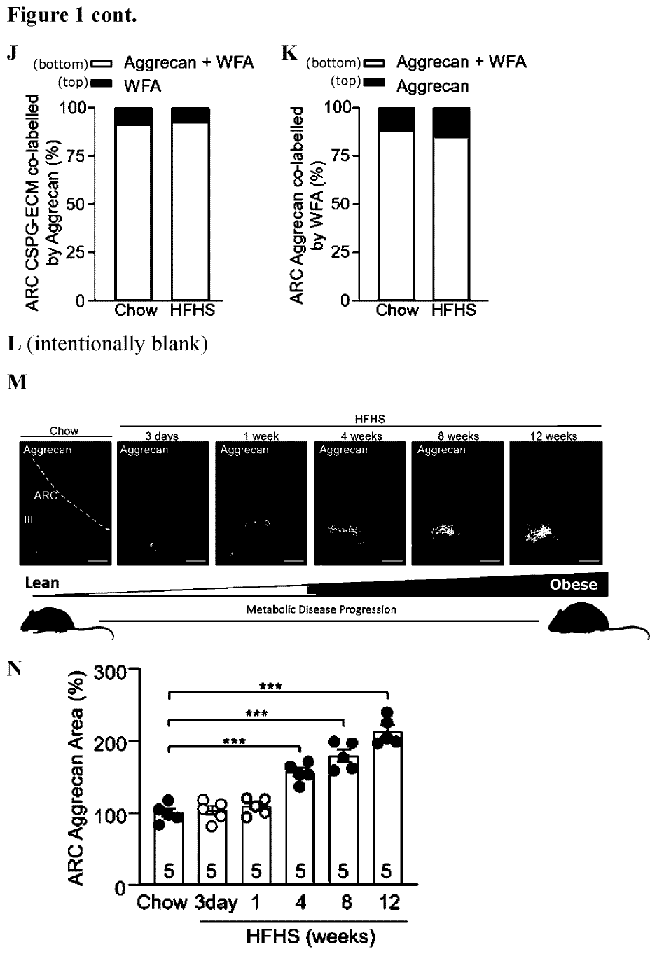

To identify the CSPG-ECM within the hypothalamus, immunostaining was performed using Wisteria floribunda agglutinin (WFA), a lectin that selectively binds to the N-acetylgalactosamine residue on chondroitin sulfate (CS) chains of the CSPG-ECM. CSPG-ECM expression was detected throughout the rostro-caudal extent of the mouse mediobasal hypothalamus (FIG. 1a-c). Strikingly prominent CSPG-ECM expression is present within the ARC (FIG. 1a-c) with notable, but significantly lower, expression within the adjacent ventromedial hypothalamus (VMH, −90.2±2.2%). CSPG-ECMs within the brain canonically surround and regulate parvalbumin cortical neurons. Within the granular retrosplenial cortex (RSG), it was observed that CSPG-ECMs surrounded 89.5±4.3% of parvalbumin neurons. However cells surrounded by the CSPG-ECM within the ARC were not parvalbumin-positive, offering a striking distinction between the CSPG-ECM present in the ARC compared to traditional CSPG-ECMs in other brain regions.

### Example 2. Neurofibrosis with the ARC Develops During the Progression of Metabolic Disease

To explore the impact of obesity on the ARC CSPG-ECM, expression was quantified in C57BL/6J mice fed a high fat-high sugar (HFHS) diet for 12 weeks, thus rendering them diet induced obese and insulin resistant. A robust increase in the area and intensity of CSPG-ECM expression throughout the rostrocaudal extent of the ARC in obese mice compared to lean, aged-matched mice (FIG. 1a-c). This finding is highly robust (n=45) and has been observed across several independent experiments. The augmentation of the CSPG-ECM following HFHS diet was not observed in the VMH (FIG. 1g-i), or RSG, suggesting that obesity driven CSPG-ECM remodelling occurs specifically within the ARC.

To explore whether the glycan composition of the ARC CSPG-ECM is also remodelled, glycomics were used to quantify chondroitin sulfate-glycosaminoglycans (CS-GAG) side chain sulfation. CS-GAG side chains are modulated by sulfotransferases that add sulfate groups to the CS-GAGs at different sites which regulates biological functions. CS-GAG sulfation occurs at either the C4 or C6 sites of N-acetylgalactosamine (CS-4S and CS-6S respectively), or the C2 position of glucuronic acid (CS-2S). CS-GAG chains can also be non-sulfated (CS-OS) or exist with combinations of sulfation patterns. It was identified that the predominant CS-GAG sulfation in the ARC is CS-4S (FIG. 1d). In the ARC of obese mice, a significant change in CS-GAG sulfation abundance was observed with elevations in ΔCS-4S, ΔCS-0S and ΔCS-2S6S with no effect on ΔCS-4S6S expression (FIG. 1d), which is consistent with the augmentation in the CSPG-ECM detected immunohistochemically.

To establish the validity of this phenomenon, CSPG-ECM expression was quantified in several independent dietary and genetic mouse models of obesity. Consistent elevations in CSPG-ECM expression were observed within the ARC of obese Sprague-Dawley rats and in obese BALB/cJ mice fed a high-fat, high-cholesterol diet. Augmentation of CSPG-ECM was also present within both monogenic (leptin receptor deficiency, db/db, and polygenic (New Zealand Obese) mouse models of metabolic disease, indicating that CSPG-ECM remodelling is observed in numerous models of obesity and metabolic disease.

The development of deleterious metabolic adaptions driving metabolic disease progression, occur following both acute and chronic HFHS diet consumption. Deficits in ARC neuronal signalling have been reported as rapidly as 72 hours following the ingestion of an obesity-promoting diet (Olofsson, et al., 2013), with abrogated tissue specific insulin resistance and elevations in adiposity occurring within one to three weeks, effects which progressively worsen over time. To identify the temporal pattern of CSPG-ECM remodelling during the progression of metabolic disease, ARC CSPG-ECM content was determined in mice fed a HFHS diet for 3 days, 1, 4, 8 and 12 weeks. Significant elevations in CSPG-ECM expression occurred within 4 weeks of HFHS feeding and were further augmented at 8 and 12 weeks (FIG. 1e,f). These effects were associated with key pathophysiological hallmarks of metabolic disease such as elevated body weight, increased adiposity and impairments in glycaemic control. The observed excessive deposition and remodelling of the CSPG-ECM within the ARC during the development of metabolic disease is a phenomenon the inventors have termed neurofibrosis.

### Example 3. Aggrecan is a Key CSPG Species Underlying Neurofibrosis in the ARC

CSPG-ECMs are comprised of four core components: 1) CS-GAG chains; 2) CSPG core proteins which are covalently bound to the CS-GAG chains; 3) a hyaluronic acid (HA) backbone; and 4) link proteins and glycoproteins that stabilise the CSPG aggregates. To explore how the composition of the ARC CSPG-ECM is remodelled in neurofibrosis, the extent to which the HA backbone changes was first determined. Using biotinylated HABP, heterogeneous staining of HA throughout the brain parenchyma with near complete WFA co-localisation in the ARC was noted (96±4.5%). Consistent with obesity promoting neurofibrosis within the ARC, the HA backbone exhibited an increase in both the staining area and intensity which is consistent with increased HA abundance observed in our ARC GAG profile (FIG. 1d). Of note, changes in ARC HA expression occurred in non-CSPG-ECM areas of the ARC which may reflect the roles of HA backbones in supporting other ECMs.

The increase of HA backbone within the ARC of diet-induced obese mice occurred in conjunction with a significant increase in abundance of hyaluronic and proteoglycan link proteins (HAPLN1) within the ARC. These link proteins serve to bind CSPGs to the HA backbone and were widely expressed throughout the hypothalamus indicating functionality in ECMs outside the ARC CSPG-ECM. Similarly, CSPG crosslinking glycoprotein, Tenascin C, also exhibited increased staining intensity within the ARC of obese mice. The increased staining intensity contributed to the ARC CSPG-ECM but Tenascin C was also expressed throughout other hypothalamic regions indicating a non-ARC CSPG-ECM specific expression.

To identify relevant CSPG's present in the ARC CSPG-ECM, versican, phosphocan, neurocan, brevican and aggrecan, major CSPG components expressed in CSPG-ECMs elsewhere in the brain, were next stained for. Whilst all CSPG components are present within the ARC to some degree, it was aggrecan (91.5±3.1% FIG. 1g,j,k) that predominantly co-localised with WFA in the ARC under both chow-fed and HFHS-fed conditions, with other CSPG components exhibiting a distinct spatial pattern from the ARC WFA labelled CSPG-ECM (FIG. 1g,jk). Obesity also promoted enhanced versican, neurocan, brevican and aggrecan expression (FIG. 1g-i) within the ARC area, however, phosphacan expression was unchanged. Furthermore, the augmented expression of aggrecan during the development of obesity occurred within a similar timeframe to that of WFA labelled CSPG-ECM (FIG. 1m,n). Taken together, these results demonstrate that obesity promotes the augmentation of most major ECM components but aggrecan is the prerogative CSPG species that underpins neurofibrosis in the ARC.

### Example 4. CSPG-ECM Tracker—A New Tool to Determine Site Specific CSPG-ECM Turnover

CSPG-ECMs have been described to exhibit slow biological turnover rates and persist in adult tissues for several months to years. The results demonstrate a comparatively rapid remodelling and augmentation of the CSPG composition in the ARC following exposure to an obesogenic diet (FIG. 1). To explain this, it was hypothesised that i) the rate of the CSPG-ECM turnover within the ARC is distinct from other brain areas and ii) the turnover rate of the CSPG-ECM within the ARC is attenuated in obesity, resulting in enhanced CSPG-ECM deposition and neurofibrosis.

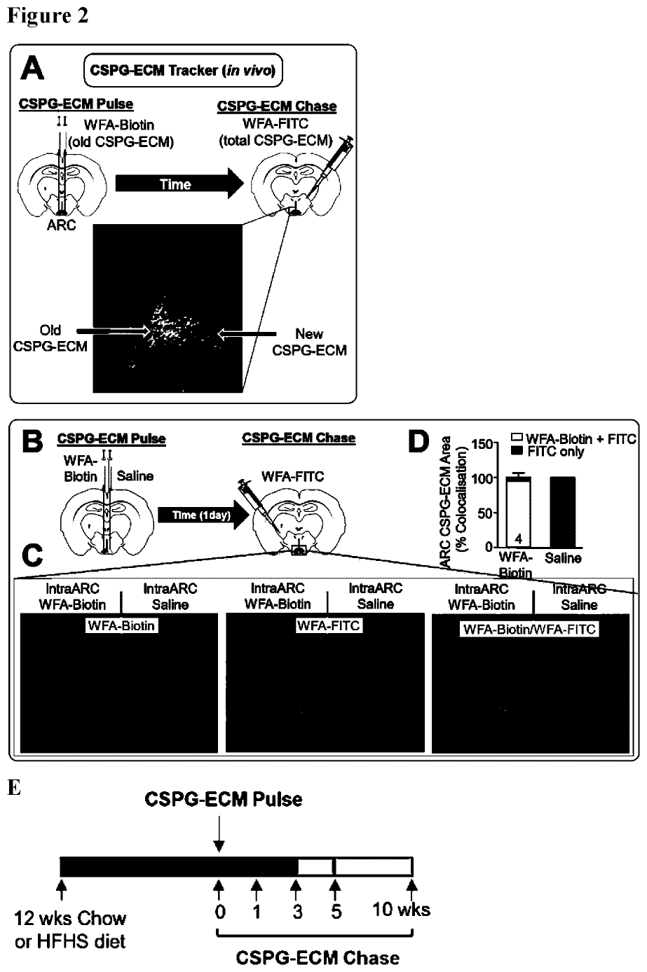

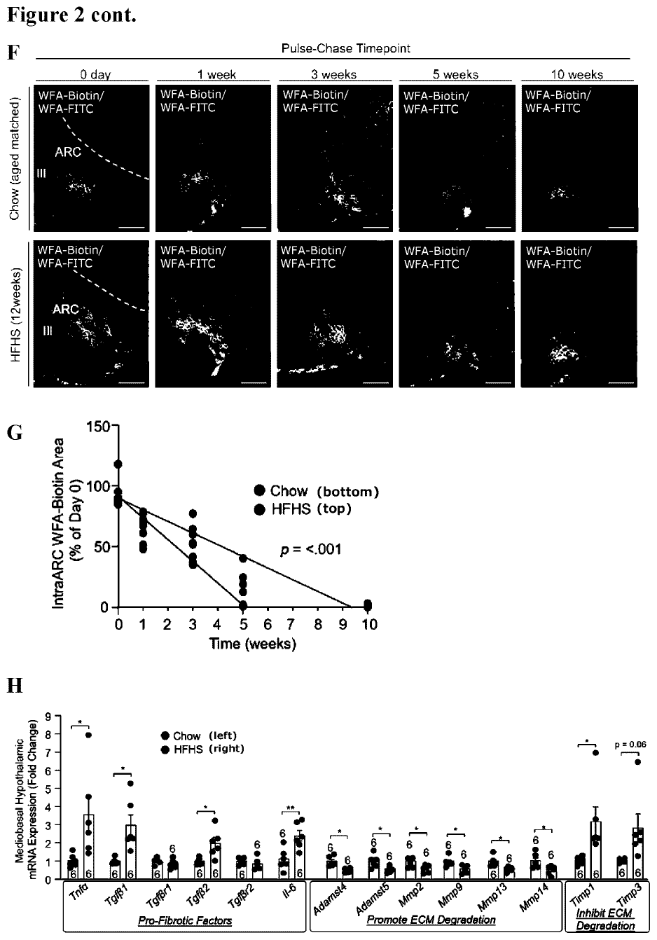

To experimentally determine the CSPG-ECM turnover rate in vivo a novel technique termed “CSPG-ECM Tracker” was developed (FIG. 2a). CSPG-ECM Tracker is a “pulse-chase” approach, utilising a stereotaxic injection of biotinylated-WFA (WFA-biotin) to “pulse” and label the CSPG-ECM in a brain region of interest. Following an in vivo incubation period, brains were extracted and processed ex vivo for the presence of WFA-biotin to “chase” the labelled CSPG-ECM remaining from the time of injection (day 0). Sections were concomitantly co-stained with WFA-FITC to reveal the total CSPG-ECM expression at the time of the “chase”. Areas of CSPG-ECM positive for WFA-biotin represent matrix still present from day 0, whereas areas expressing only WFA-FITC indicate novel matrix synthesised post day 0 (FIG. 2a).

To validate this approach as a bone fide tracker of CSPG-ECM turnover, the extent to which intraARC injected WFA-biotin faithfully labels the CSPG-ECM within the ARC was first determined (FIG. 2b-d). To do this, the ARC of adult chow-fed mice was unilaterally “pulsed” with either WFA-biotin or saline and chased expression one day later (FIG. 2b,c). Using this approach, near-complete co-expression of pulsed WFA-biotin with WFA-FITC (total CSPG-ECM) was identified, suggesting that CSPG-ECM Tracker faithfully labels CSPG-ECMs in the ARC in vivo (FIG. 2d). Speckles of WFA positive signal outside the ARC represents WFA-biotin leakage into the circulation and binding to CSPG-ECM expressed in blood vessels (FIG. 2c).

To validate if the “pulsed” WFA-biotin signal faithfully binds and labels CSPG-ECM present only at the time of injection and not the re-binding of free WFA-biotin to newly synthesised CSPG-ECM, the ARC of adult chow-fed mice was bilaterally pulsed with WFA-biotin and three days later digested the CSPG-ECM in the ARC with chondroitinase ABC (chABC), an enzyme that specifically digests CSPG-ECM. Enzymatic digestion of the “pulsed” WFA-biotin bound CSPG-ECM was completely abolished following chABC treatment compared to vehicle, indicating that WFA-biotin is binding to CSPG-ECM components present only at the time of the pulse injection. CSPG-ECM Tracker represents the first viable method to assess in vivo turnover of CSPG-ECM in a brain region specific manner.

### Example 5. The ARC CSPG-ECM Exhibits a Dynamic and Rapid Turnover Rate

To determine the basal CSPG-ECM turnover within the ARC, WFA-biotin was “pulsed” into the ARC of adult chow-fed mice and “chased” its expression at 0, 1-, 3-, 5-, and 10-weeks post-injection (FIG. 2e). Using CSPG-ECM Tracker it was identified that CSPG-ECM within the ARC of chow fed C57BL/J mice exhibited a 5-week turnover period, characterised by temporal reductions in CSPG expression at 1- and 3-weeks post injection (FIG. 2f,g). To assess if CSPG-ECM turnover is consistent in other brain regions, WFA-biotin was “pulsed” into the RSG of adult chow-fed mice and “chased” its expression at 0- and 5-weeks post-injection. Unlike within the ARC, the CSPG-ECM in the RSG was still present at five weeks, albeit reduced by 61%. Significant CSPG-ECM expression was further identified within blood vessels in the adjacent corpus callosum that exhibited no degradation within the 5 week post-injection period. These results demonstrate that the ARC exhibits a uniquely rapid degradation rate of CSPG-ECM and establishes a precedent for differential ECM turnover throughout the brain.

### Example 6. Attenuated CSPG-ECM Turnover in Obesity Drives Neurofibrosis

It was hypothesised that neurofibrosis in the ARC is attributed to attenuated CSPG-ECM degradation. To test this, WFA-biotin was “pulsed” into the ARC of obese mice and “chased” its expression at 0, 1-, 2-, 5-, and 10-weeks post-injection (FIG. 2e,f). As expected, higher CSPG-ECM expression was present in the ARC of obese compared to lean age-matched controls, recapitulating the above findings that obesity drives neurofibrosis in the ARC (FIG. 1). It was determined that the CSPG-ECM degradation rate in the ARC of obese mice was significantly reduced compared to that of lean mice (lean=2.6%/day vs obese=0.1%/day, FIG. 2g). This attenuation in CSPG-ECM turnover resulted in the presence of WFA-biotin in the ARC up to 10 weeks post injection which is double that seen in lean mice (5 weeks). These results identify that neurofibrosis is driven by attenuated CSPG-ECM degradation and illustrates the remarkable transfiguration of the CSPG-ECM in the ARC during the development of metabolic disease.

To further elucidate the molecular mechanism underlying obesity-driven alterations in the CSPG-ECM turnover, the gene expression of established ECM synthesis/degradation enzymes within the mediobasal hypothalamus of lean versus obese mice was quantified. ECM composition and remodelling are tightly governed by the balance of matrix metalloproteinases (MMPs), proteolytic enzymes known to degrade ECMs, and their inhibitors, tissue inhibitors of metalloproteinases (TIMPs). A significant reduction in the expression of several key ECM proteases (Adamst4, Adamst5, Mmp2, Mmp9, Mmp13, Mmp14) was observed within the mediobasal hypothalamus of obese mice (FIG. 2h). Conversely, elevated expression of TIMPs (Timp1 and Timp3) was also observed, which may promote neurofibrosis through the inhibition of MMPs (FIG. 2h). Elevated expression of pro-fibrotic inflammatory factors Tnfα, Tgfβ1, Tgfβ2, Tgfβr1, Tgfβr2 and 16 was additionally observed (FIG. 2h), which are established regulators of fibrosis in peripheral tissues.

### Example 7. Neurofibrosis Occurs Around AgRP Neurons within the ARC

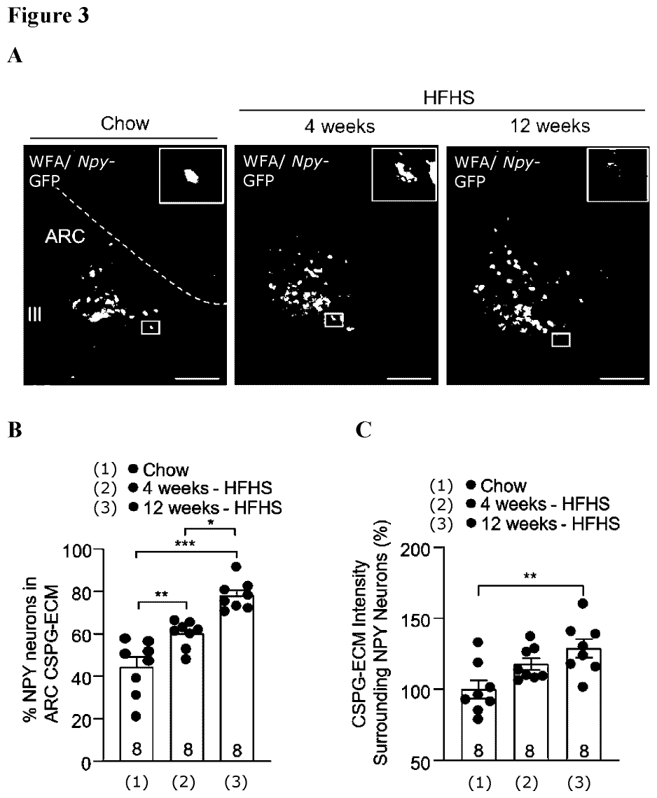

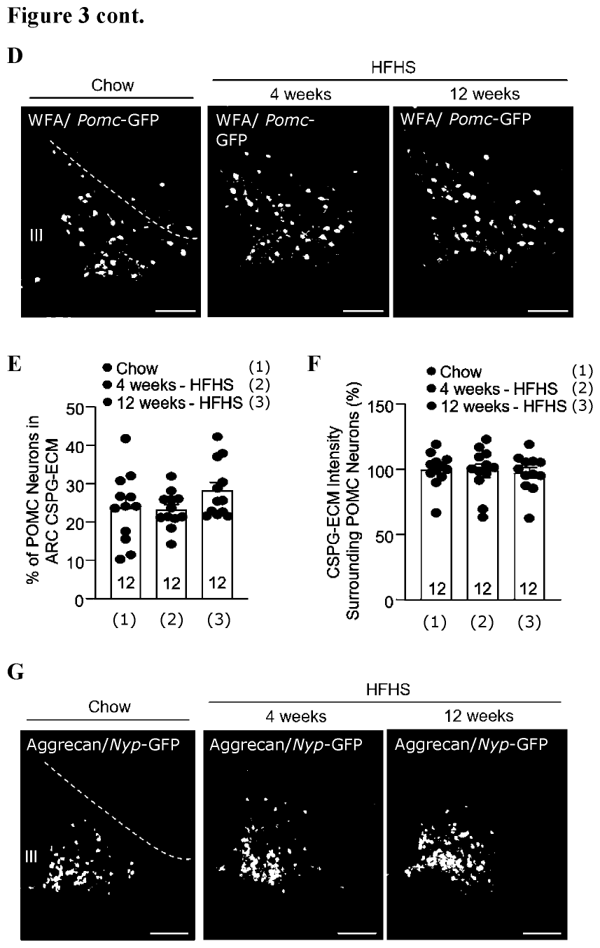

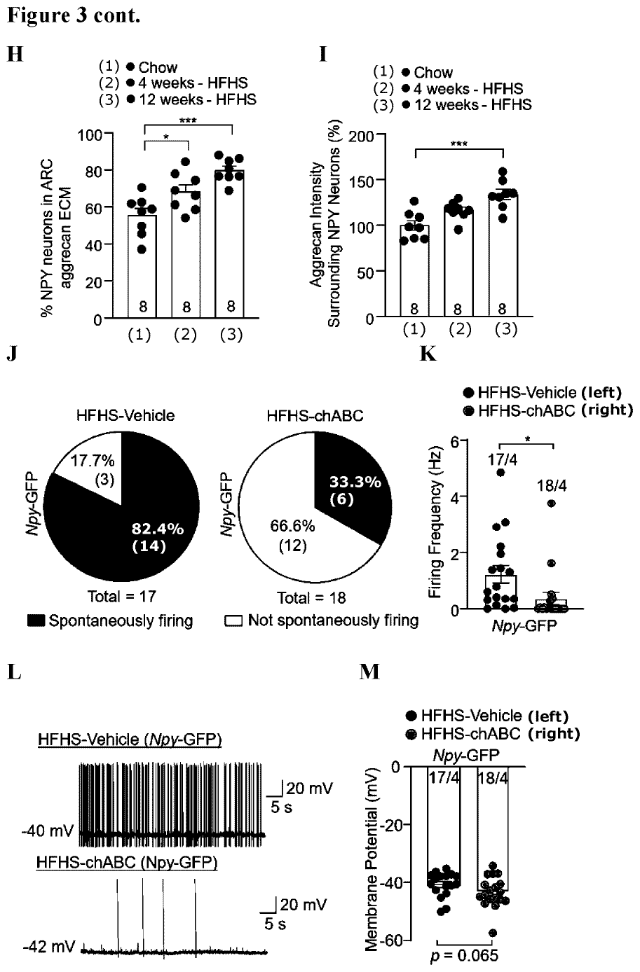

The ARC contains two metabolically relevant neuronal populations termed agouti-related peptide (AgRP) neurons and pro-opiomelanocortin (POMC) neurons. AgRP and POMC neurons are well established neuronal populations within the ARC critical to the regulation of metabolism and play central roles in the development of metabolic disease. Using Npy-GFP (to mark AgRP neurons) and Pomc-GFP mice, it was identified that under chow-fed conditions, 44%±13% of AgRP neurons (FIG. 3a,b) and 24%±9% of POMC neurons (FIG. 3d,e) in the ARC were ensheathed within the CSPG-ECM. It was determined that significantly more AgRP (60%±6%, FIG. 3a,b) but not POMC neurons (23%±5%, FIG. 3d-e) become ensheathed within the CSPG-ECM following 4 weeks of HFHS feeding. By 12 weeks of HFHS feeding a further recruitment of AgRP neurons (78%±7%; FIG. 3a,b) was noted, alongside an enhancement of surrounding WFA staining (FIG. 3c), effects that were absent around POMC neurons (FIG. 3f) and independent of changes in neuron numbers.

Earlier, it was identified that enhanced abundance of aggrecan is a compositional hallmark of neurofibrosis within the ARC (FIG. 1g-m). Consistent with this, aggrecan-positive CSPG-ECM was detected surrounding AgRP neurons in chow fed mice (FIG. 3g-i). As expected, aggrecan-positive CSPG-ECM ensheathed AgRP neurons to a similar extent as WFA and exhibited a similar recruitment of AgRP during the development of neurofibrosis (FIG. 3g-i). Collectively, these results demonstrate that neurofibrosis develops around metabolically relevant AgRP neurons in the ARC during the development of metabolic disease.

Obesity influences the intrinsic excitability of AgRP neurons, as both firing rate and resting membrane potential are elevated following extended high fat feeding. Furthermore, removal of CSPG-ECM expressed around cortical and brainstem neurons reduces membrane excitability as evidenced by decreases in firing frequency. Accordingly, the impact of CSPG-ECM recruitment on AgRP neuronal function was investigated using whole cell patch clamp electrophysiology. At 12 weeks of HFHS feeding over 82% of AgRP neurons spontaneously fired (FIG. 3j), whereas when the CSPG-ECM within the ARC was disassembled with chABC, spontaneous firing was reduced to 33% (FIG. 3j). Consistent with this significant reduction in firing frequency (FIG. 3k,l), a trend toward a reduction in resting membrane potential was also observed (FIG. 3m, p=0.065), supporting a role for the ARC CSPG-ECM in regulating the intrinsic electrophysiological properties of AgRP neurons.

### Example 8. Abrogating ARC Neurofibrosis Protects Against Obesity

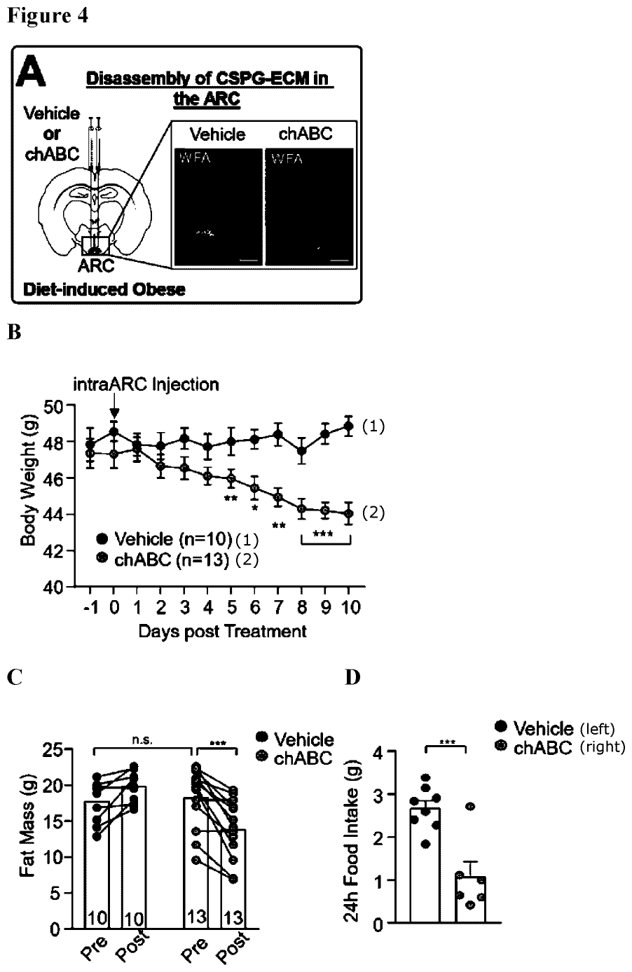

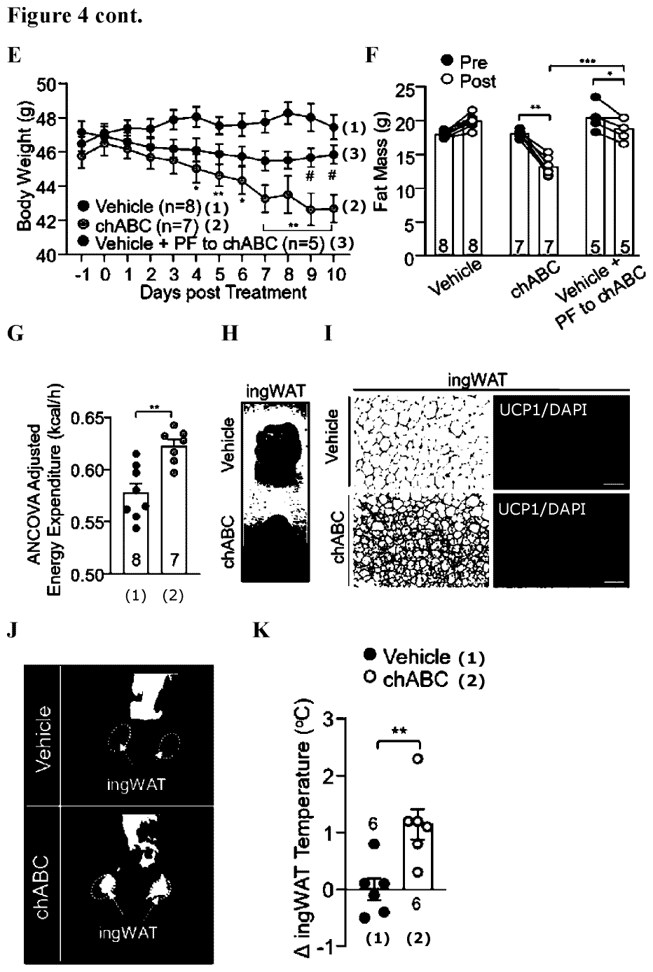

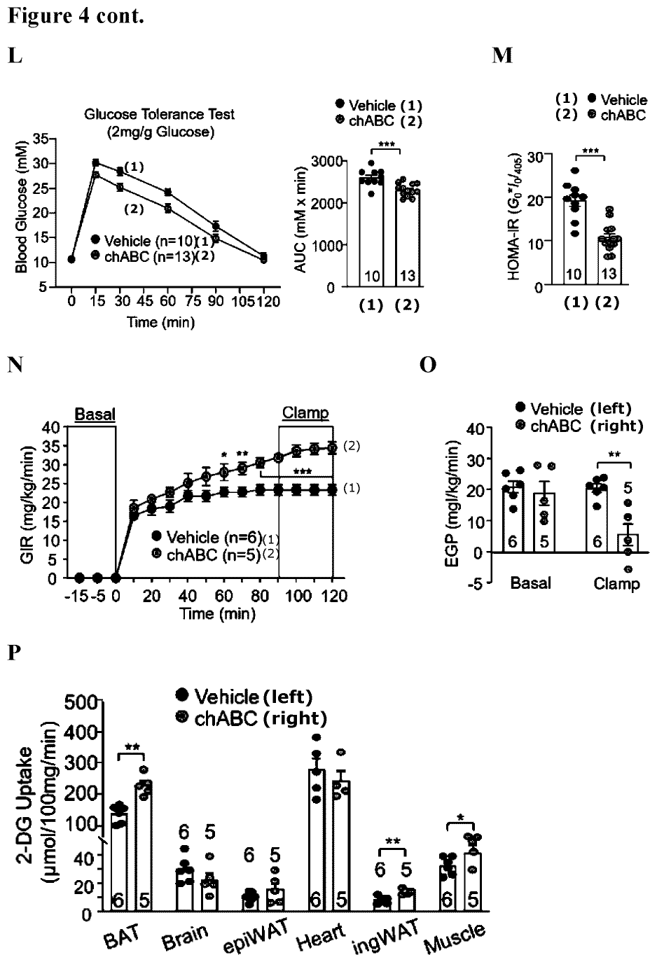

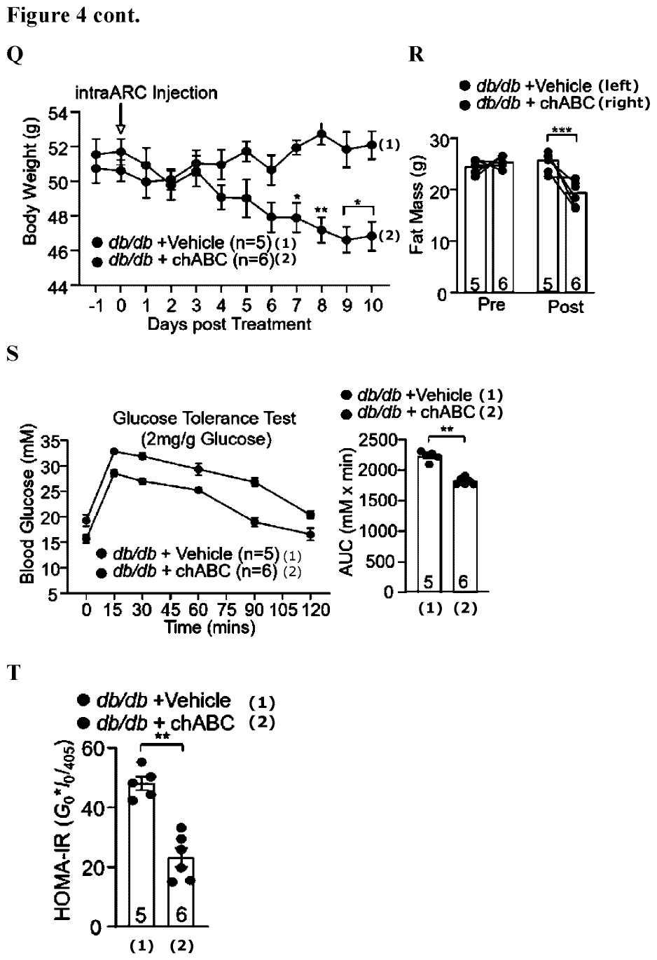

Obesity is characterised by increased adiposity and impaired glycaemic control, effects that are driven by hyperphagia, reduced adaptive thermogenesis and insulin resistance. The functional contribution of ARC neurofibrosis to the development of metabolic disease remains unknown. To address this, the CSPG-ECM was selectively disassembled within the ARC of obese HFHS-fed mice using chABC. The intraARC delivery of chABC markedly reduced the expression of CSPG-ECM (FIG. 4a) within the ARC. Remarkably, disassembly of the ARC CSPG-ECM in obese mice promoted progressive and substantial weight loss (FIG. 4b), which was mediated by a marked reduction in adiposity (24±16%; FIG. 4c). The changes in body mass and composition were due, in part, to a profound reduction in caloric intake as mice lacking the CPSG-ECM in the ARC consumed significantly less food than HFHS-fed controls (FIG. 4d). Neurofibrosis abrogation regulates food intake through the promotion of satiety without the induction of non-food specific adverse behaviours such as nausea, excessive grooming, or sedation.

To explore the extent to which the repression of feeding contributes to weight loss, vehicle treated mice were pair-fed, so that they consumed the same amount of food as mice lacking the CSPG-ECM in the ARC and assessed effects on body weight and adiposity. Pair-feeding resulted in weight loss (FIG. 4e) and decreased fat mass (FIG. 4f), albeit not to the extent observed in ad libitum chABC treated mice (FIG. 4e,f). This suggests that the chABC mediated repression of food intake partially accounts for the effects on weight loss. Notwithstanding these effects, a significant difference remained between pair-fed vehicle compared to ad libitum fed chABC treated mice indicating that caloric intake alone cannot explain the difference in body weight and may include a contribution from energy expenditure (FIG. 4g). Using indirect calorimetry to measure energy expenditure it was found that mice lacking the CSPG-ECM in the ARC exhibited elevated whole body energy expenditure and oxygen consumption without only effects on substrate partitioning or ambulatory activity.

Consistent with elevations in energy expenditure, a dramatic upregulation of adipose tissue thermogenesis following intraARC chABC treatment was also observed in both inguinal white adipose tissue (ingWAT) and brown adipose tissue (BAT) depots (FIG. 4h-k). Disassembly of CSPG-ECM in the ARC of diet induced obese mice, was accompanied by an increase in ingWAT browning, as assessed by (1) gross morphology (FIG. 4h) and histology, monitoring for the presence of multilocular lipid droplets (FIG. 4i); (2) immunofluorescence, monitoring for UCP-1 positive adipocytes (FIG. 4i); and (3) enhanced dermal ingWAT temperature in vivo (FIG. 4j,k). Moreover, chABC treatment also enhanced dermal BAT temperature, indicating a substantial activation of adaptive thermogenesis.

Abrogating ARC neurofibrosis was also accompanied by a marked improvement in glucose homeostasis, as assessed by decreased glucose excursions in glucose tolerance tests (FIG. 4l) and reduced fasting blood glucose alongside a reduction in circulating insulin and HOMA-IR (homeostatic model assessment of insulin resistance) index as a measure of whole-body insulin resistance (FIG. 4m). Importantly these tests were conducted 4 days post chABC before significant effects on body weight were present, indicating a direct action of neurofibrosis abrogation on glucose metabolism independent of changes in body weight.

To further explore the role of ARC neurofibrosis in the development of whole-body insulin resistance, whole-body insulin sensitivity and glycaemic control was assessed using hyperinsulinemic-euglycemic clamps in weight matched mice. The glucose infusion rate (GIR) required to maintain euglycemia during the clamp was markedly increased in intraARC chABC treated mice, consistent with abrogated neurofibrosis within the ARC improving whole-body insulin sensitivity (FIG. 4n). The improved insulin sensitivity was mediated by an increased repression of endogenous glucose production (FIG. 4o) and an increased glucose disposal rate, respective measures of hepatic gluconeogenesis and skeletal glucose uptake. Enhanced glucose uptake was observed in skeletal muscle, however, no effect was present in epididymal white adipose tissue, heart or brain (FIG. 4p). It was previously shown that ARC neurons coordinate glucose metabolism through thermogenesis occurring in ingWAT and BAT. Consistent with this regulation, disassembly of neurofibrosis within the ARC promoted glucose uptake in BAT and ingWAT (FIG. 4p). The beneficial effects of chABC intraARC injection on body weight, adiposity, glycaemic control, and insulin resistance were also recapitulated in genetically severely obese and type-2 diabetic db/dh mice treated with chABC (FIG. 4q-t), further substantiating the causal role for ARC neurofibrosis in the development of metabolic disease.

Taken together, these results demonstrate a profound and unprecedented causal role for the ECM within the brain in the development of metabolic disease. These studies causally link the generation of neurofibrosis within the ARC to the progression and maintenance of metabolic disease through the development of hyperphagia, whole-body insulin resistance, increased adiposity, and impaired adaptive thermogenesis. Remarkably, disassembly of neurofibrosis within the ARC confers weight loss and a reversal of key metabolic disease phenotypes.

### Example 9. Neurofibrosis Impedes Insulin Entry into the ARC and Promotes Neuronal Insulin Resistance

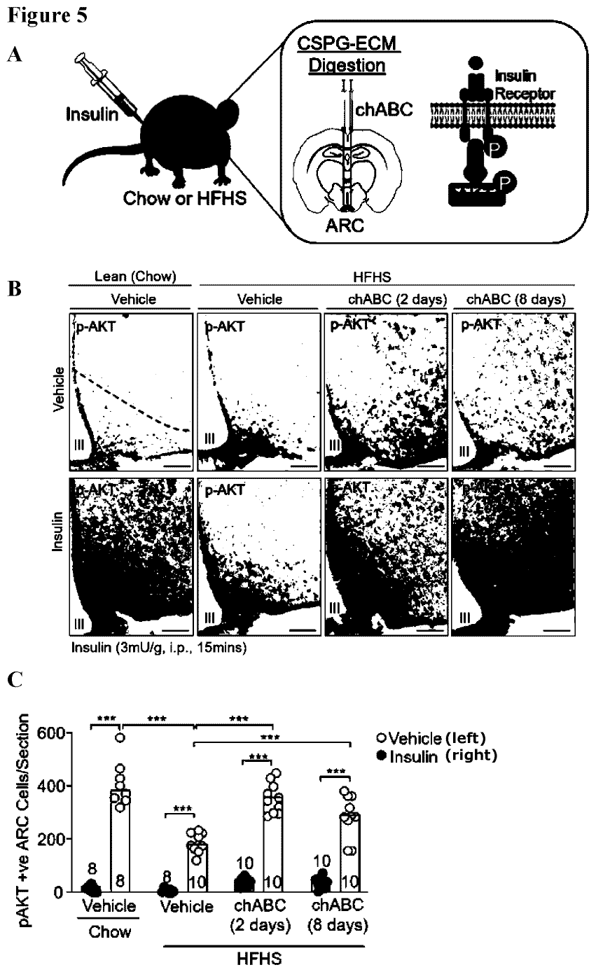

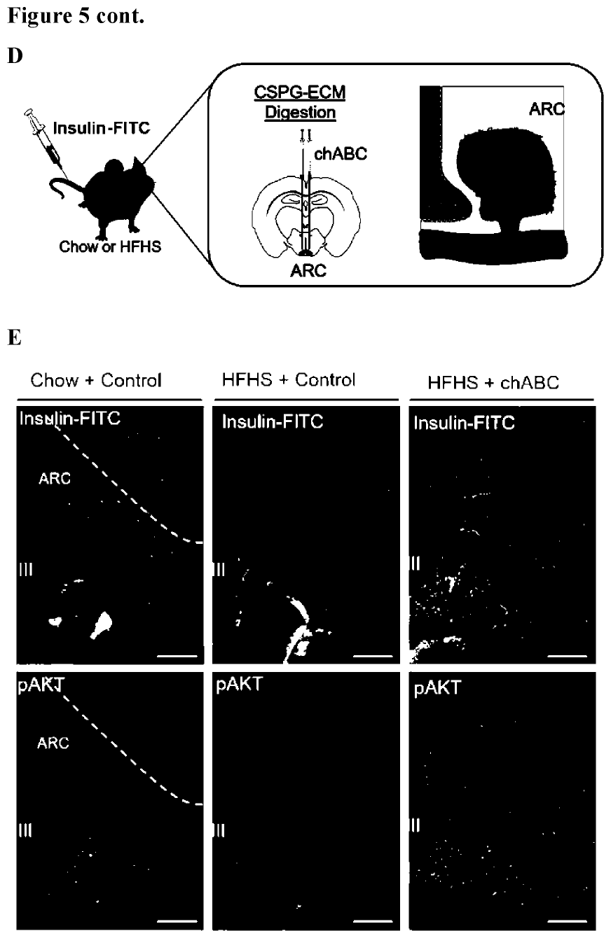

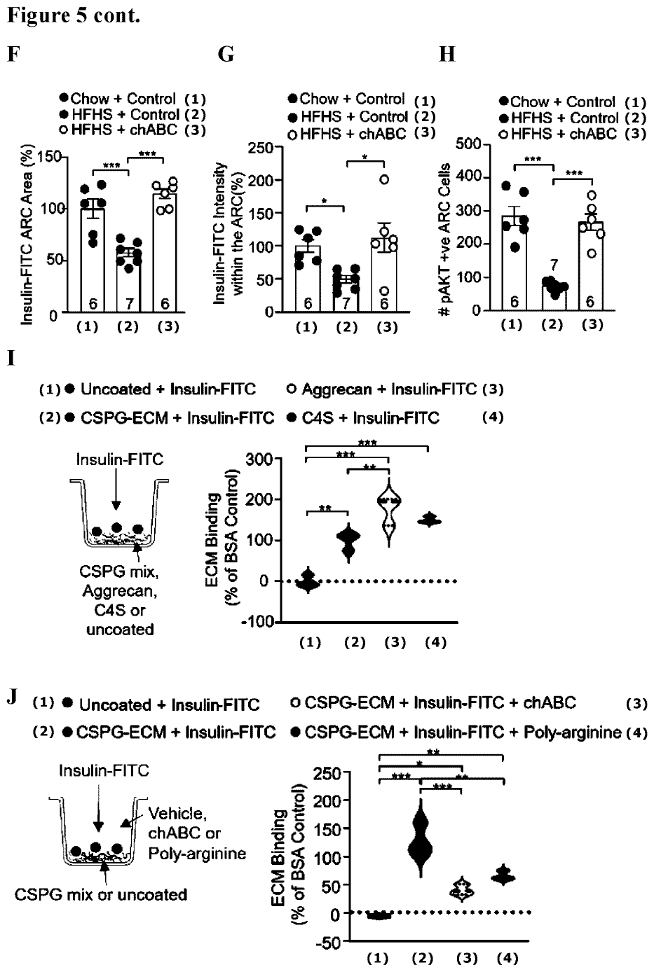

Given the association between fibrosis and insulin resistance in peripheral tissues, it was hypothesised that accumulated CSPG-ECM may impede the delivery of insulin from the circulation into the ARC, and this may represent a new mechanism underlying insulin resistance. To explore this, vehicle or chABC was bilaterally administered into the ARC of 12-week HFHS-fed obese and chow-fed mice and assessed effects on insulin receptor activation (FIG. 5a). As expected, systemic insulin administration in chow mice induced robust expression of the phosphorylation of AKT (p-AKT) within the ARC. This effect was markedly attenuated in obese mice, demonstrating that obesity drives the development of insulin resistance within the ARC (FIG. 5b,c). Disassembly of the ARC CSPG-ECM in obese mice rapidly ameliorated insulin resistance within ARC neurons as shown by the re-instatement of both p-AKT (FIG. 5b,c) and insulin receptor phosphorylation within the ARC two days (before any effects on body weight occurred), and eight days post chABC injection (FIG. 5b,c).

CSPG-ECMs within the brain regulate neuronal function by physically impeding access and interaction of extracellular molecules with the target cell. As such, it was surmised that obesity driven neurofibrosis within the ARC CSPG-ECM may mechanistically promote insulin resistance by impeding the access of insulin to neurons within the ARC. To explore this, fluorescein isothiocyanate tagged insulin (insulin-FITC) was peripherally administered and the entry and signalling of insulin in the ARC in lean versus obese mice was quantified (FIG. 5d). Robust insulin-FITC appearance and internalisation (FIG. 5e-g) ensuing p-AKT signalling in the ARC of lean mice was observed, confirming circulating insulin enters and signals directly to cells in the ARC (FIG. 5e-h). The entry of insulin-FITC was impaired in obese mice (FIG. 5e-g), an effect that was accompanied by a commensurate reduction in insulin-induced p-AKT signalling (FIG. 5e,h). Remarkably. CSPG-ECM disassembly in the ARC of obese mice reinstated insulin entry into the ARC, similar to that observed in lean mice (FIG. 5e-h), and in turn restored insulin-induced p-AKT signalling (FIG. 5e-h).

To examine if the neurofibrotic impedance of insulin transport is mediated by the CSPG-ECM surrounding the blood brain barrier (BBB), insulin-FITC was infused into the cerebral spinal fluid, thus bypassing the BBB. Here insulin-FITC entered the ARC of lean mice, an effect drastically attenuated in obese mice and rescued following CSPG-ECM disassembly. Taken together, these results demonstrate that the neurofibrotic CSPG-ECM within the ARC, and not within the BBB, obstructs insulin entry into the ARC to promote insulin resistance. CSPG-ECM regulation of insulin-FITC access into the ARC is an insulin specific effect.

To provide mechanistic insight into how neurofibrosis impedes insulins infiltration and signalling within the ARC we performed an in vitro binding assay to assess insulin-ECM interactions. Insulin-FITC was incubated for 2-hours on plates coated with either mixed CSPGs (aggrecan, neurocan, phosphacan and versican), or specific core CSPG-ECM components that compose neurofibrosis (aggrecan or chondroitin 4-sulfate) (FIG. 5i). A specific dose-dependent binding of insulin-FITC to mixed CSPGs, aggrecan and C4S was observed, effects that were abolished in the presence of chABC (FIG. 5j). To mechanistically explain the interaction between ECM and insulin, it was hypothesized that the highly negative charge of sulfated GAGs attached to the CSPGs impede ligand-receptor binding. To explore this, insulin-FITC was incubated with a CSPG mix in the presence of poly-arginine, a positively charged peptide that neutralizes the negative charges of CSPGs. It was found that insulin-ECM interactions were significantly reduced in the presence of poly-arginine, indicating that the negative charges of GAGs in the CSPG-ECM also regulates insulin-ECM interactions (FIG. 5j).

Putative potassium (K+) currents were examined in AgRP neurons in the ARC. To determine whether the improved capacity of insulin to access and signal to ARC neurons underlies the neurofibrosis meditated modulation of AgRP membrane excitability (FIG. 3j-m), whole cell electrophysiology was performed. Activation of K+ channels on AgRP neurons is known to hyperpolarise the resting membrane potential resulting in reduced firing rates. To directly address the potential role of neurofibrosis to modulate K+ currents in AgRP neurons, current-voltage relationships were examined in the presence of tetrodotoxin. Coinciding with a reduction in firing rate and membrane potential in AgRP neurons of diet-induced obese mice following neurofibrosis removal (FIG. 3j-m), an upward shift in the current-voltage curve was observed, indicative of enhanced K+ currents. To determine the contribution of improved insulin signalling following neurofibrosis removal in the modulation of K+ currents, a gene expression analysis was conducted in the mediobasal hypothalamus for several K+ channels known to be present in AgRP/Npy neurons. The upregulation of several K+ channels following ARC CSPG-ECM digestion in diet-induced obese mice was demonstrated. To determine whether these changes are due to the improved ability for insulin to access these neurons and modulate K+ channel activity, an insulin receptor antagonist (S961) was utlized. It was demonstrated that the upregulation of K+ channels following neurofibrosis abrogation is attenuated by S961, revealing an insulin receptor dependent regulation of neuronal activity following ARC CSPG-ECM digestion.

Overall, these findings demonstrate that the ARC CSPG-ECM directly interacts with insulin, and the development of neurofibrosis promotes insulin resistance and AgRP excitability through the impairment of insulins ability to access and signal to key ARC neuronal populations.

### Example 10. ARC Neurofibrosis Promotes Metabolic Disease Through Dysfunctional AgRP Insulin Signalling

As neurofibrosis occurs specifically around AgRP neurons (FIG. 4) it was hypothesised that impaired insulin signalling within AgRP neurons is the likely cell type underlying these effects. To determine whether neurofibrosis development around AgRP neurons drives alterations in the neuronal circuitry governing metabolism, the expression of AgRP peptide was examined within ARC terminals projecting to the paraventricular hypothalamus (PVH). This ARC AgRP to PVH circuit is a well-established output of AgRP neurons to regulate metabolism and glycemic control. It was observed that AgRP peptide expression innervating the PVH is significantly elevated in obese mice compared to lean mice, an effect that was reversed upon neurofibrosis attenuation. This reduction in AgRP peptide expression and subsequent reduction in AgRP inhibitory tone to the melanocortin circuitry of the PVH may explain how neurofibrosis around AgRP neurons may propagate metabolic dysfunction.

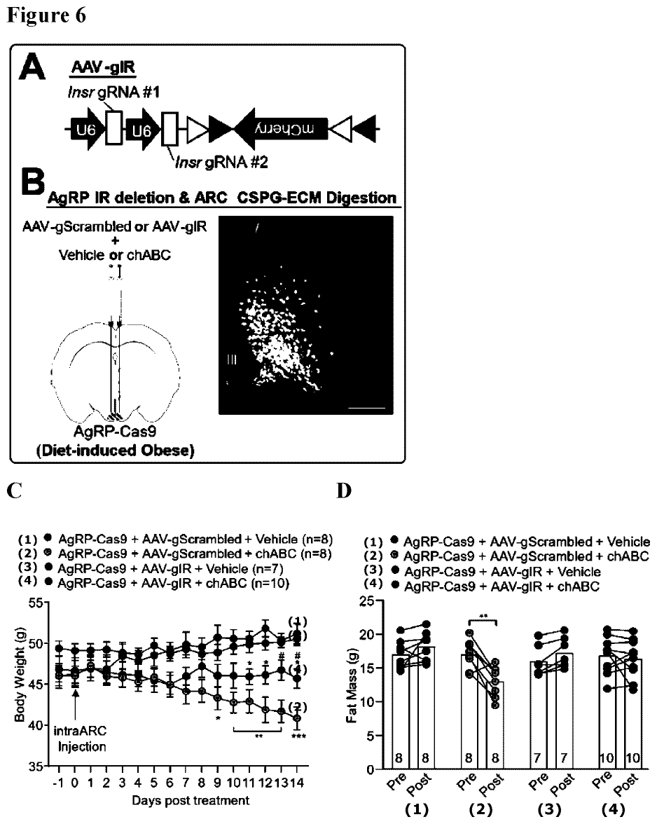

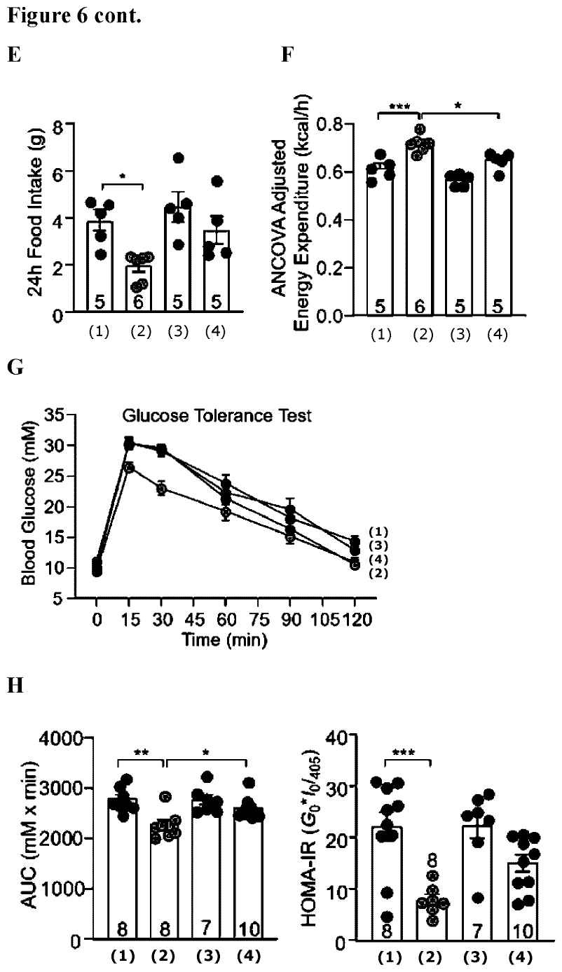

To define the causative role of neurofibrosis in driving impaired AgRP insulin signaling, a mouse model capable of conditional AgRP neuronal insulin receptor deletion in the adult diet-induced obese state was generated. CRISPR gene-editing was employed and two guide RNA's (sgRNA) targeting proximal regions of exon 2 of the mouse insulin receptor (InsR) gene were identified. In the presence of Cas9 endonuclease, these sgRNA's excised a substantial −82 bp region of InsR exon 2, resulting in a near complete ablation of IR protein expression. An AAV expressing the two IR sgRNA sequences alongside a Cre-dependent mCherry to report AAV-transduced neurons was then constructed (gIR-AAV, FIG. 6a). To target CRISPR-mediated excision of the IR in AgRP neurons, Agrp-IRES-Cre was crossed with Rosa26-LSL-Cas9-GFP knock-in mice to generate AgRP-Cas9 (Agrp-IRES-Cre; Rosa26-LSL-Cas9-GFP) mice, which specifically expressed Cas9 and GFP in AgRP neurons. To examine the efficacy of in vivo CRISPR-mediated disruption of InsR in AgRP neurons, AAV-gIR or a scrambled sgRNA control AAV (AAV-gScrambled) were bilaterally injected into the ARC of 12-week-old adult AgRP-Cas9 mice. Successful CRISPR mediated disruption of InsR was confirmed by the presence of a ~419 bp PCR product (ΔInsr^{CRISPR}, ~82 bp smaller than wt ~501 bp) in the mediobasal hypothalami of AgRP-Cas9 mice. CRISPR-mediated disruption of IR in AgRP neurons led to impaired insulin signalling which further validated effective, AgRP specific disruption of IR expression.

To define the contribution of AgRP IR signalling to neurofibrosis attenuation effects on whole body metabolism, AAV-gIR or AAV-Scrambled were bilaterally injected into the ARC of obese 12-week obese AgRP-Cas9 mice (FIG. 6b). One-week later mice received bilateral intraARC administration of chABC or vehicle to disassemble neurofibrosis within the ARC. Recapitulating previous findings, chABC treatment in the ARC of diet-induced control (AAV-gScrambled) AgRP-Cas9 mice promoted weight loss (FIG. 6c), reduced adiposity (FIG. 6d), reduced caloric intake (FIG. 6e), enhanced energy expenditure (FIG. 6f), and improved glycaemic control (FIG. 6g,h). Remarkably, all the actions on whole-body metabolism were dependent upon functional insulin receptor signalling in AgRP neurons as they were significantly attenuated in chABC treated AAV-IR AgRP-Cas9 mice (FIG. 6c-h). Taken together, obesity driven neurofibrosis promotes the development of metabolic disease via impaired insulin signalling in AgRP neurons. Moreover, degradation of ARC neurofibrosis improves whole-body metabolism and glycaenic control, at least in part, through the re-instatement of insulin receptor signalling within AgRP neurons.

### Example 11. Pharmacological Attenuation of Neurofibrosis Promotes Weight Loss in Obesity

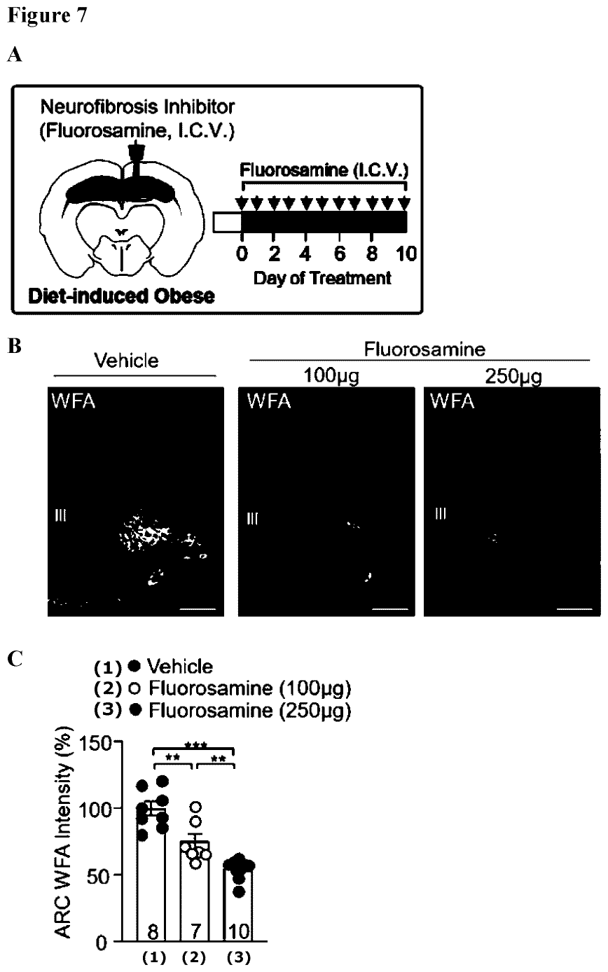

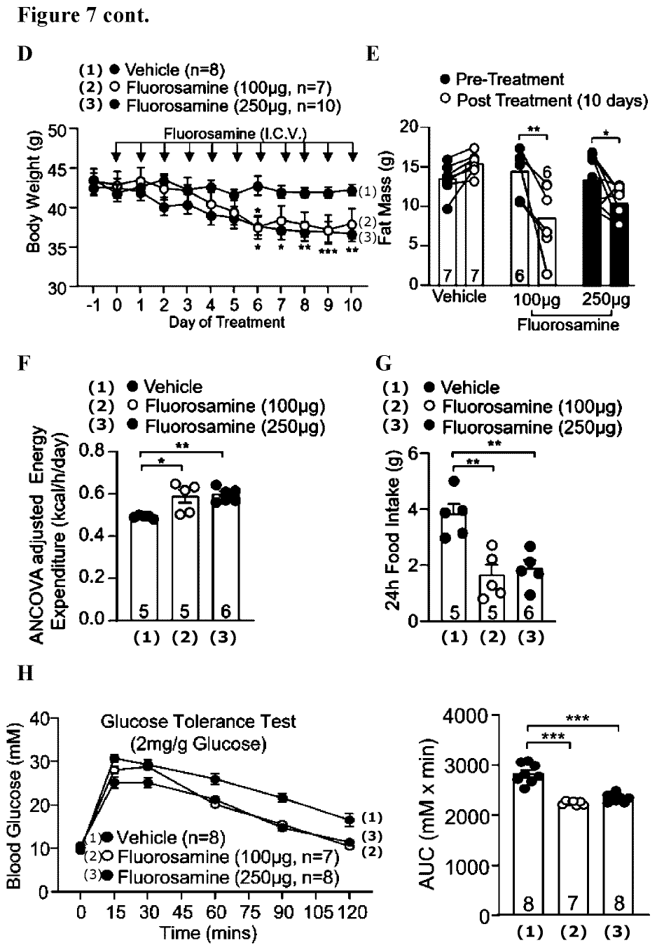

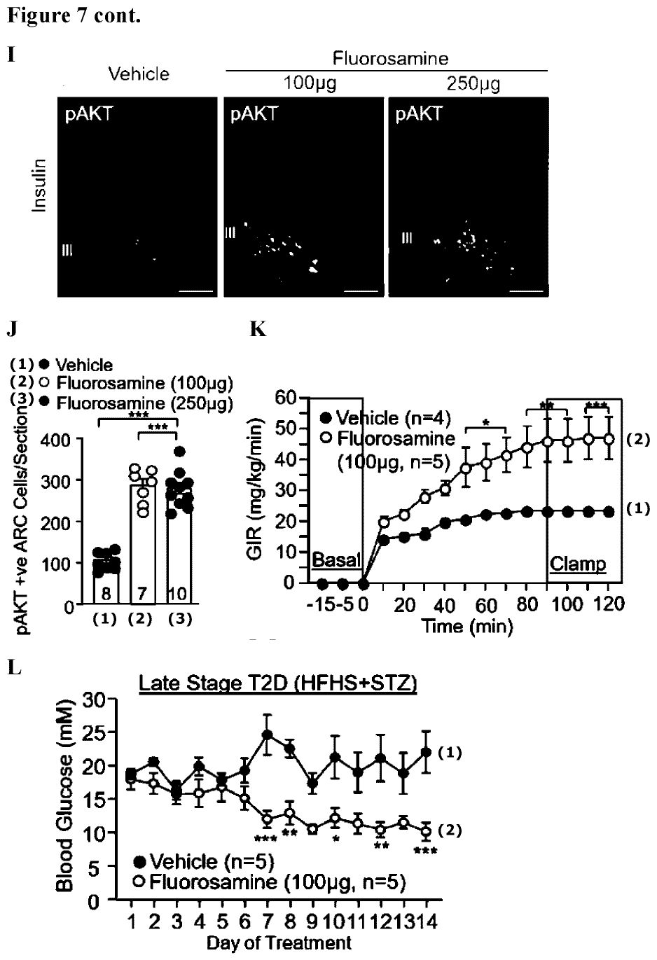

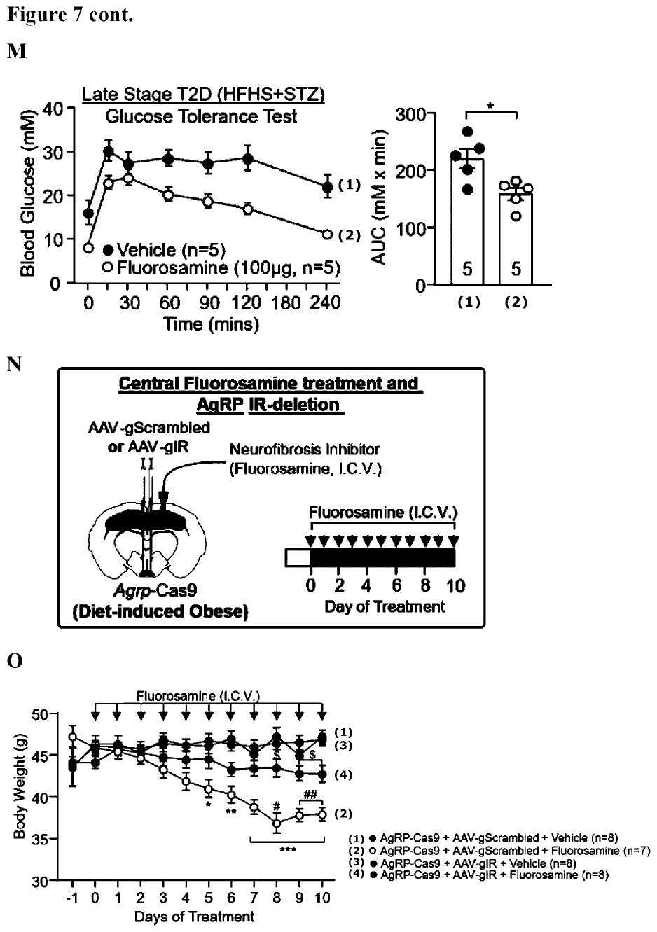

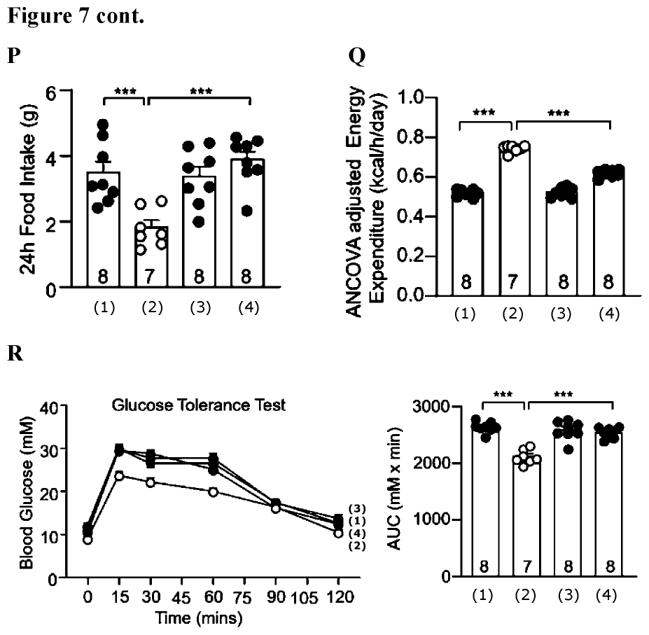

Targeting the ECM surrounding metabolic neurocircuitry, instead of the cells themselves, offers a unique therapeutic strategy. A major therapeutic challenge in targeting ECMs lies in the development of small molecule inhibitors capable of regressing fibrotic ECM. Whilst the enzyme chABC is effective at digesting CSPG-ECM and ameliorating neurofibrosis when injected into discrete brain areas, its enzymatic activity is rapidly exhausted at body temperature. Thus, its therapeutic capability is limited. To explore the pharmacological viability of targeting neurofibrosis within the brain, a recently characterised small molecule inhibitor, fluorosamine (per-O-acetylated-4-F-N-acetylglucosamine), was used. Fluorosamine is a competitive inhibitor of 4-epimerase, an enzyme essential for creating the nucleotide sugar substrate UDP-N-acetylgalactosamine, which is required for to the assembly and elongation of CS-GAG chains on CSPGs. To directly target the brain, fluorosamine was delivered intracerebroventricularly (I.C.V., circumventing actions on peripheral tissues) to obese mice for 10 days (FIG. 7a). Using WFA immunostaining it was observed that fluorosamine treatment significantly attenuated neurofibrosis within the ARC (FIG. 7b,c). Central fluorosamine administration did not reduce CSPG-ECM in other brain areas such as the RSG cortex and at the highest dose only partially attenuated expression in the habenula, an effect that may be explained by the rapid turnover rate of CSPGs in ARC. Consistent with enzymatic CSPG-ECM disassembly in the ARC, central fluorosamine treatment promoted weight loss (FIG. 7d), reduced adiposity (FIG. 7e), enhanced energy expenditure independently of ambulatory activity (FIG. 7f), supressed food intake through enhanced satiety (FIG. 7g), and improved glucose tolerance (FIG. 7h). Moreover, fluorosamine treatment enhanced insulin induced p-AKT signalling in the ARC indicating a significant reinstatement of ARC insulin sensitivity (FIG. 7i,j). Mechanistically, fluorosamine improved glycaemic control through the enhancement of whole-body insulin sensitivity (FIG. 7k), hepatic glucose production, and tissue specific glucose uptake, namely in skeletal muscle, BAT and ingWAT, as assessed using hyperinsulinemic-euglycemic clamps in weight matched obese mice. The utility and capacity of fluorosamine treatment to promote the remission of metabolic disease was also observed in a mouse model of late-stage type-2 diabetes (HFHS plus low dose streptozotocin treatment, FIG. 7l,m), further substantiating the utility of targeting neurofibrosis to treat distinct stages of T2D progression.

Whilst flurosoamine and chABC are mechanistically distinct in how they dissemble CSPG-ECM, fluorosamine phenocopies chABC's action on metabolism. In line with this, the extent to which fluorosamine actions on metabolism are mediated through AgRP insulin receptor signalling was explored. To address this, AAV-gIR or AAV-gScrambled was bilaterally injected into the ARC of obese 12-week obese AgRP-Cas9 mice and one-week later delivered vehicle or fluorosamine (100 μg/animal I.C.V.) daily for 10 days (FIG. 7n). Recapitulating previous findings, fluorosamine treatment to control AAV-gScrambled AgRP-Cas9 mice promoted weight loss (FIG. 7o), reduced adiposity, attenuated caloric intake (FIG. 7p), enhanced energy expenditure independently of ambulatory activity (FIG. 7q), and improved glycaemic control (FIG. 7r). These positive metabolic outcomes of fluorosamine treatment were mitigated, at least in part, in AAV-IR AgRP-Cas9 mice, demonstrating the requirement for insulin receptor signalling in AgRP neurons to mediate the sequalae of metabolic benefits induced by fluorosamine (FIG. 7o-r).

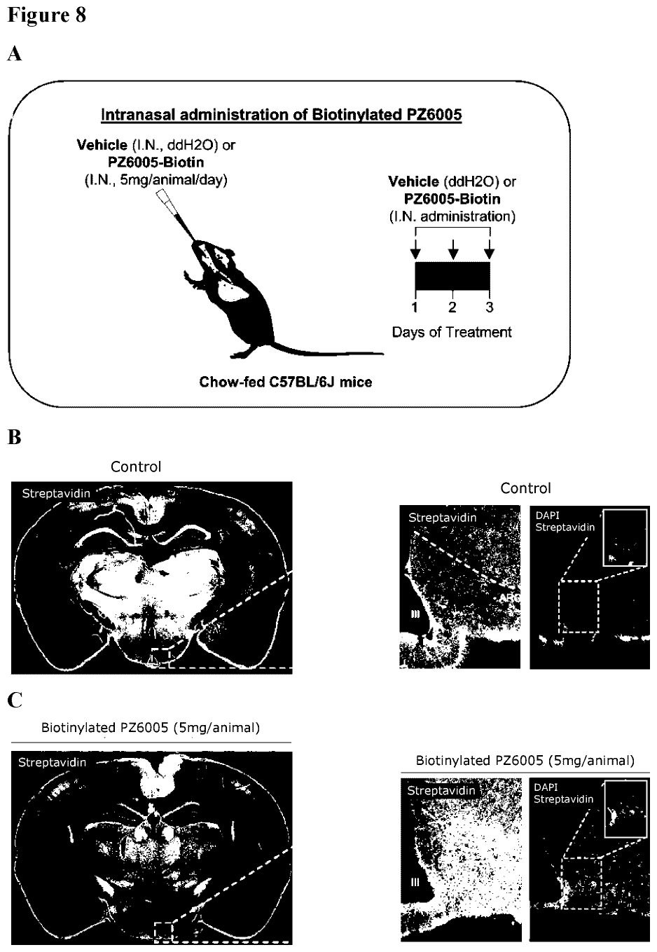

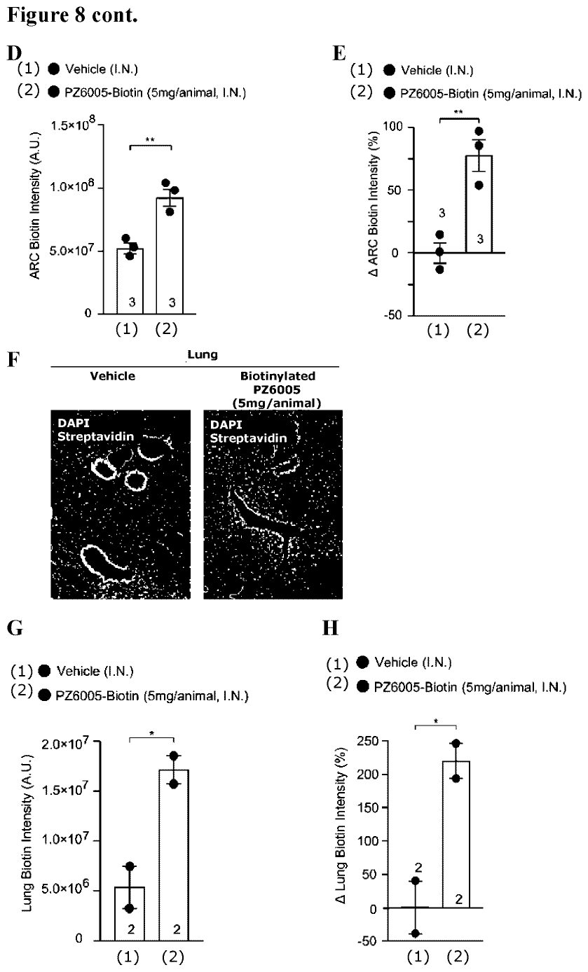

To facilitate therapeutic translation to humans, intranasal delivery of fluorosamine was explored as a possible route of administration to ensure targeted delivery of neurofibrosis inhibitors to the brain. To determine whether neurofibrosis inhibitors can successfully be delivered intranasally, a biotin-conjugated fluorosamine molecule to C57BL/6J mice was intranasally administered and its biodistribution throughout the brain determined (FIG. 8a,b). Abundant fluorosamine accumulation was detected within the brain with significant accumulation within the ARC (FIG. 8b), demonstrating successful delivery of fluorosamine to the site of neurofibrosis. To determine the efficacy of brain-targeted delivery of neurofibrosis inhibitors for the treatment of metabolic disease, fluorosamine was intranasally delivered to diet-induced obese mice for 14 days (FIG. 8c). Intranasal delivery of fluorosamine successfully attenuated ARC neurofibrosis (FIG. 8d,e) and recapitulated the whole-body metabolic improvements observed with intracerebroventricular delivery (FIG. 8f-l). These effects are likely mediated by enhanced insulin signalling to neurons within the ARC (FIG. 8m,n).

Taken together, these results further substantiate the role of neurofibrosis in the development of central insulin resistance and whole-body metabolic dysfunction.

### Example 12. Intranasal Administration Delivers Biotin-Conjugated Fluorosamine (PZ6005) to the Brain

PZ6005 conjugated with biotin (PZ6005-biotin) or PZ6005-unconjugated (FIG. 8a) was intranasally delivered to mice for 3 days, and biotin-streptavidin signals were subsequently quantified. High biotin was expressed in the ARC in PZ6005-biotin treatment compared to the controls (FIG. 8b-e). Significant biotin expression in the lung of PZ6005-biotin-treated mice was also observed (FIG. 8f-h). However, this was to a much lesser extent to that seen in the ARC (quantification). These results suggest that PZ6005-biotin had ability to be delivered through intranasal administration to the ARC and lung.

### Example 13. Intranasal Administration of Fluorosamine (PZ6005) Attenuates CSPG-ECM Expression within the ARC

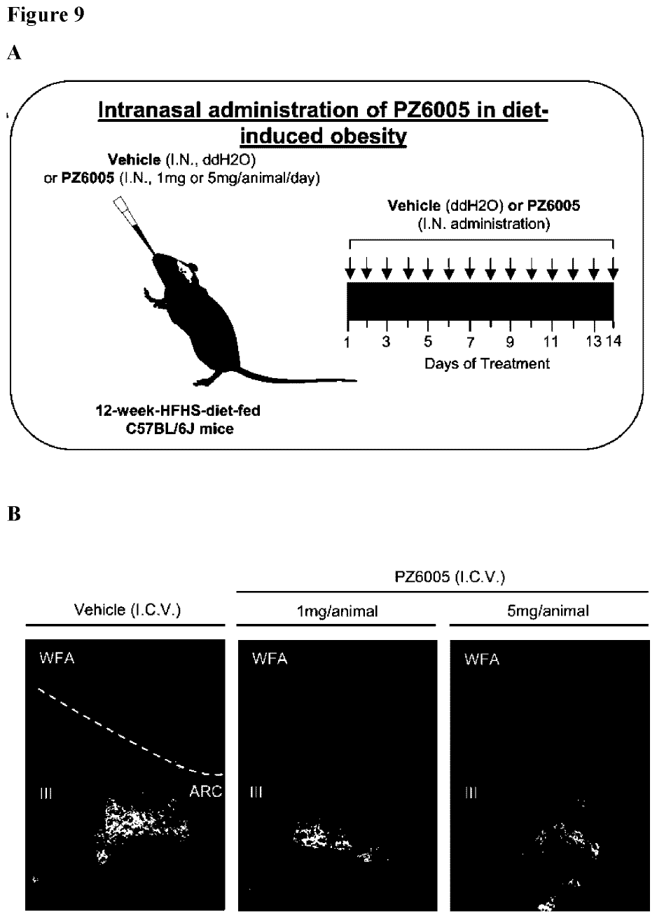

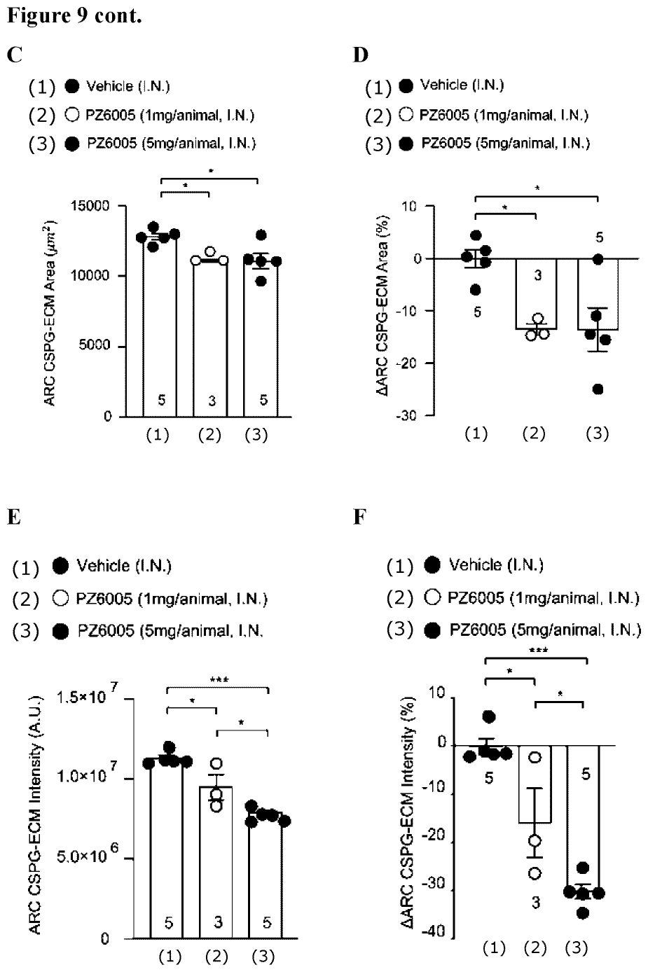

To determine whether intranasally administered PZ60005 suppresses ARC CSPG-ECM expression, 12-week-HFHS-diet-fed obese mice were subjected to intranasal administration of either vehicle or PZ6005 (1 or 5 mg/animal/day) for 14 days (FIG. 9a), and quantified WFA-immunostaining CSPG-ECM expression within the ARC (FIG. 9b). Both doses of intranasal PZ6005 treatment robustly diminished the area and intensity of ARC CSPG-ECM. CSPG-ECM area of 1 mg and 5 mg treatment group was 13.54±1.03% and 13.59±4.09% lower than the controls (FIG. 9c,d). The intensity was decreased by 15.94±7.17% in the 1 mg treatment and was further reduced by 30.16±1.52% in the highest dose of intranasal PZ6005 treatment (FIG. 9e,f). These results demonstrate that intranasal administration of PZ60005 dose-dependently decreases ARC CSPG-ECM expression and attenuates ARC neurofibrosis driven by obesity.

### Example 14. Therapeutic Abrogation of ARC Neurofibrosis Using Intranasal Fluorosamine (PZ6005) Promotes the Reduction in Body Weight Alongside Diminished Hyperadiposity

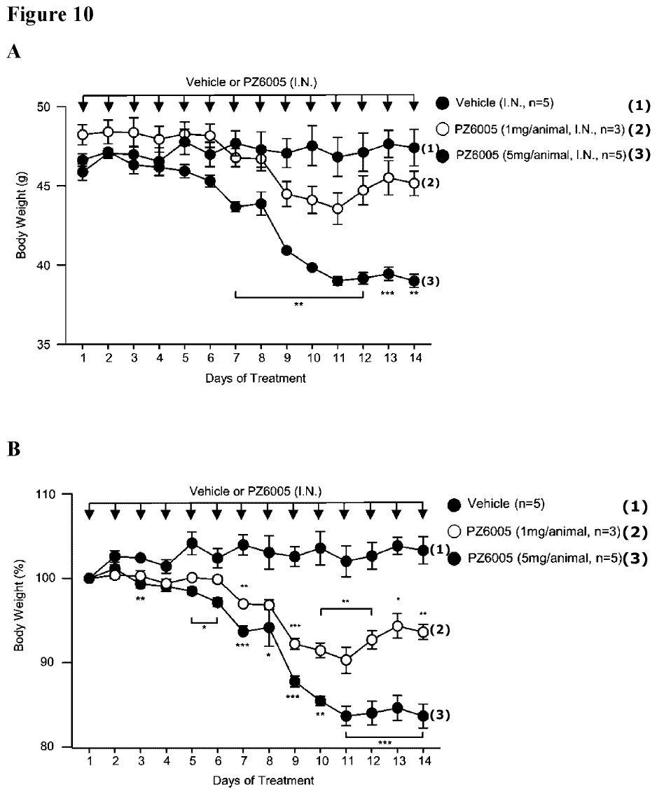

Changes in body weight of 12-week-HFHS-diet-fed obese mice during 14 days of intranasal administration of either vehicle or PZ6005 (1 or 5 mg/animal/day). Intranasal PZ6005 treatment induce weight loss in a dose-dependent manner, as assessed by significant differences in weight changes across groups. The 1 mg and 5 mg treatment groups had robustly decreased weight from day 7 and day 3, respectively. The 5 mg treatment began to significantly reduce more weight than the 1 mg treatment on day 9 and persisted to day 14. At the end of the experiment, mice in the highest dose of PZ6005 treatment had lost 6.36±0.88% and those in the 1 mg treatment had lost 16.32±1.41%, whereas control littermates maintained at 3.57±1.28% of body weight (FIG. 10a).

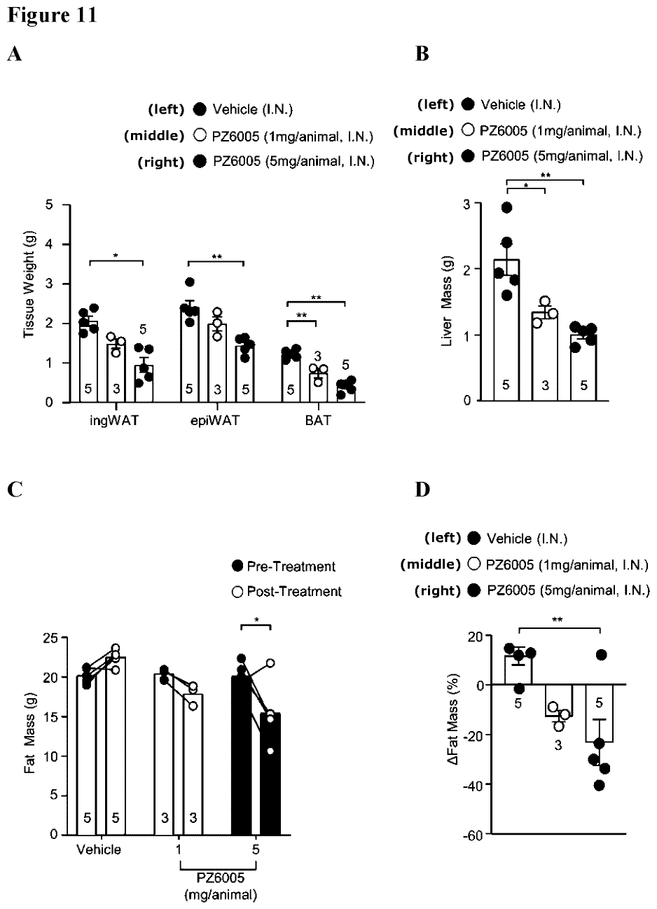

To determine the influence of intranasal PZ6005-mediated attenuated neurofibrosis on tissue-specific adiposity and body composition, peripheral tissues and fat mass of the mice were weighed following the 14-day administration. The mass of epiWAT, BAT and liver was decreased in the 1 mg treatment, while highest dose of intranasal PZ6005 remarkedly reduced tissue mass (FIG. 11a,b). Moreover, the reduction in overall fat mass depended upon the doses of PZ6005. Post-treatment fat mass was decreased by 12.59±2.29% in the 1 mg treatment and was further diminished by 23.21±9.27% in the 5 mg treatment (FIG. 11c,d), compared to the pre-treatment mass. These results suggest that intranasal delivery of PZ6005 is associated with a dose-dependent weight loss accompanied by improved tissue-specific hyperadiposity and reduced fat mass.

### Example 15. Therapeutic Abrogation of ARC Neurofibrosis Using Intranasal Fluorosamine (PZ6005) Dose-Dependently Improves Glucose Homeostasis Alongside Enhanced Insulin Sensitivity

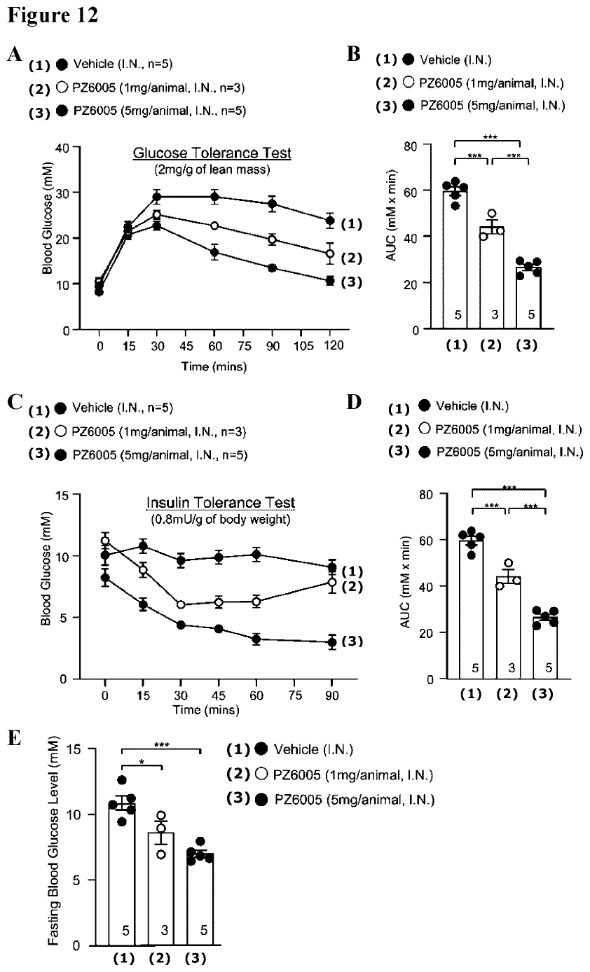

Obese mice intranasally receiving either vehicle or PZ6005 (1 or 5 mg/animal/day) were subjected to I.P. GTT after 6 hrs fasting period and I.P. insulin tolerance test (ITT) after 4 hrs fasting period. Decreased glucose excursion in both GTT and ITT revealed that intranasal PZ6005-treated mice had better glucose tolerance than control littermates (FIG. 12a,b), in company with an enhanced insulin sensitivity (FIG. 12c,d). Moreover, after 60 mins of I.P. glucose injection in GTT and after 0 mins of I.P. insulin injection, the 5 mg PZ6005 treatment showed a significantly higher glucose clearance rate than that of the 1 mg treatment group and the controls (FIG. 12a-d). Reduction in fasting (12 hrs) glucose level further substantiated that both doses of intranasal PZ6005 treatment improved glycaemic control (FIG. 12e).

These results demonstrate that intranasal delivery of PZ6005 enhances whole-body insulin sensitivity to improve glucose homeostasis in a dose-dependent manner.

### Example 16. Therapeutic Abrogation of ARC Neurofibrosis Using Intranasal Fluorosamine (PZ6005) Improves Insulin Receptor Signalling within the ARC

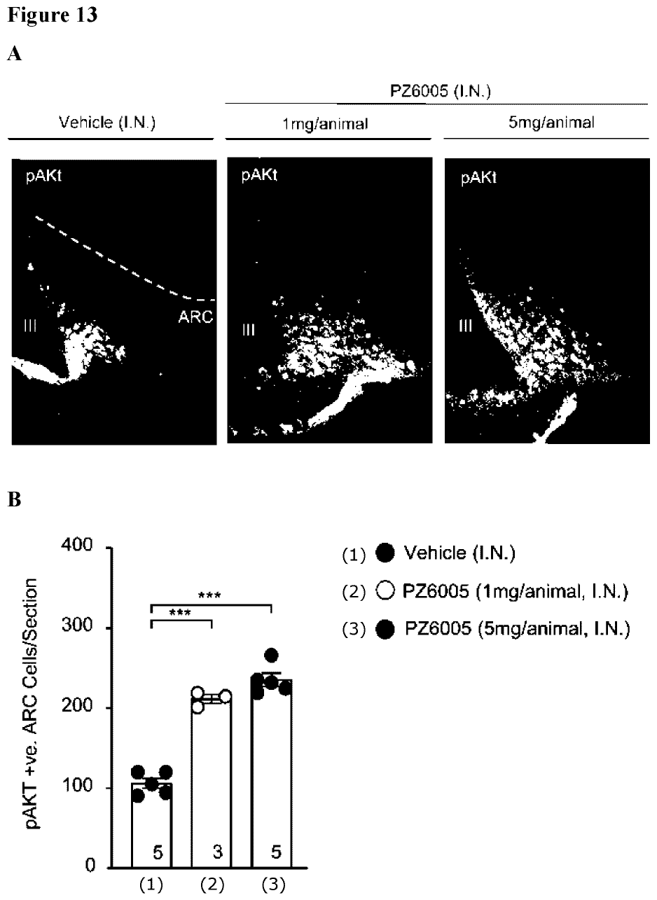

ARC pAKT +ve. cells of obese mice receiving 14-day intranasal administration of either vehicle or PZ6005 (1 or 5 mg/animal/day) were examined to demonstrate the extent to which intranasal PZ6005-mediated attenuation of CSPG-ECM influence insulin receptor signalling within the ARC (FIG. 13a). The ARC in both doses of intranasal PZ6005 treatment had enhanced insulin receptor signalling, as assessed by the robust increase in ARC pAKT +ve. cells (FIG. 13b). This result indicates that intranasally administered PZ6005 as a neurofibrosis inhibitor enhances insulin sensitivity of ARC parenchyma.

### Example 17. Intermittent Administration of PZ6005 Attenuates CSPG-ECM Expression and Sustains this Suppression Over Time

To determine whether therapeutic attenuation of ARC neurofibrosis using PZ6005 would enhance liraglutide-mediated improvement in both energy and glucose metabolism, 12-week-HFHS-diet-fed obese C57BL/6j mice were I.C.V. administered with either vehicle or PZ6005 every other day for the first 14 days of a 28-day treatment; meanwhile, the mice received daily subcutaneous (S.C.) injections of either vehicle or liraglutide during this treatment (FIG. 14a).

The efficacy of every-second-day PZ6005 injection on targeting neurofibrosis was assessed through quantifying WFA-immunostained CSPG-ECM expression within the ARC (FIG. 14b). Single liraglutide treatment showed no effects on altering the CSPG-ECM. By contrast, a significant reduction in the area and intensity of ARC CSPG-ECM was observed in the mice receiving PZ6005 treatment. CSPG-ECM area of PZ6005 and liraglutide/PZ6005 treatment group were 31.56±3.73% and 32.39±6.77% lower than liraglutide group, respectively (FIG. 14c,d). ARC CSPG-ECM intensity was reduced by 22.65±7.851% and 37.32±5.143% following PZ6005 treatment and co-administration of liraglutide and PZ6005, compared with the mice receiving liraglutide (FIG. 3.4.1e,f). The mice had not received PZ6005 for the last 14 days of the treatment, and their ARC CSPG-ECM expression in the mice was kept lower than those of mice receiving vehicle or liraglutide alone.

These results demonstrate that every-second-day administration of PZ6005 is effective for suppressing ARC neurofibrosis and this effect could be maintained for the long term, compared with controls and liraglutide treatment.

### Example 18. Therapeutic Attenuation of ARC Neurofibrosis Restores Neuronal Insulin Receptor Signalling as Well as Enhancing GLP-1 Sensitivity

The extent to which (co) administration of liraglutide and/or PZ6005 influences hormone signalling in the ARC parenchyma was assessed following the 28-day liraglutide and/or PZ6005 treatment of Example 17 by I.P. delivering a bolus of insulin and liraglutide to the mice.

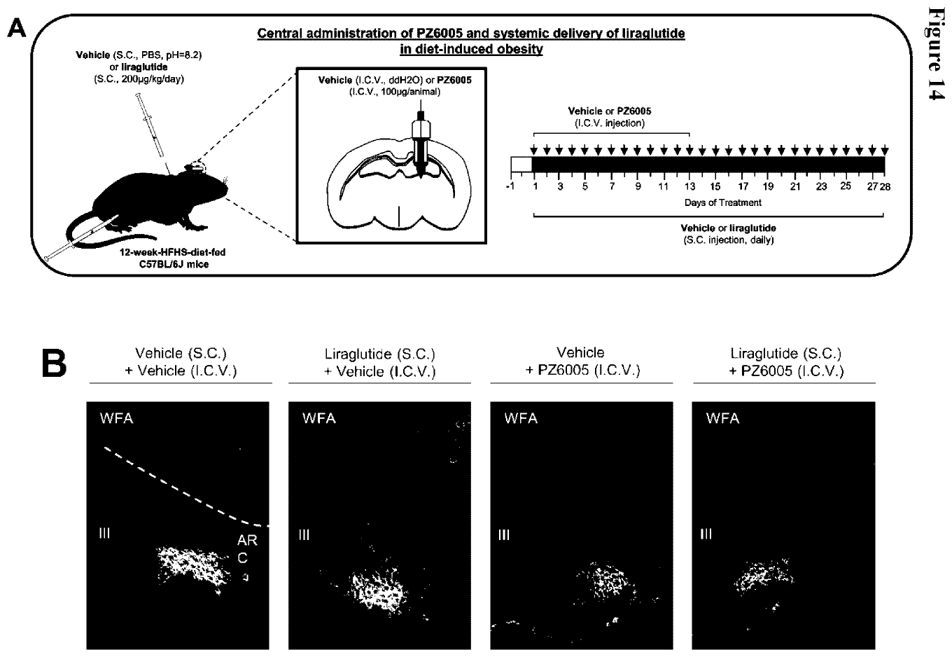

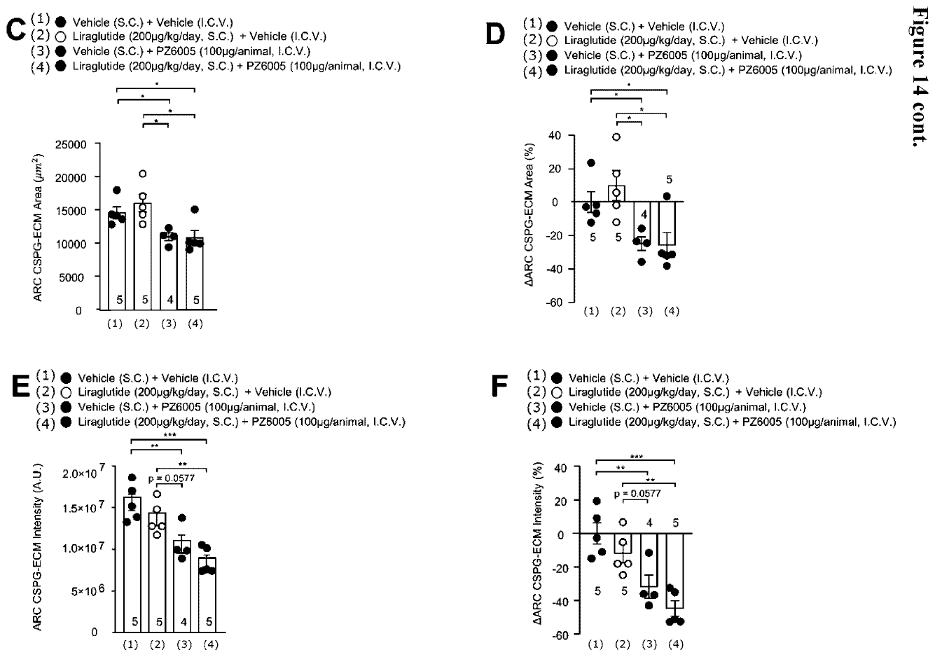

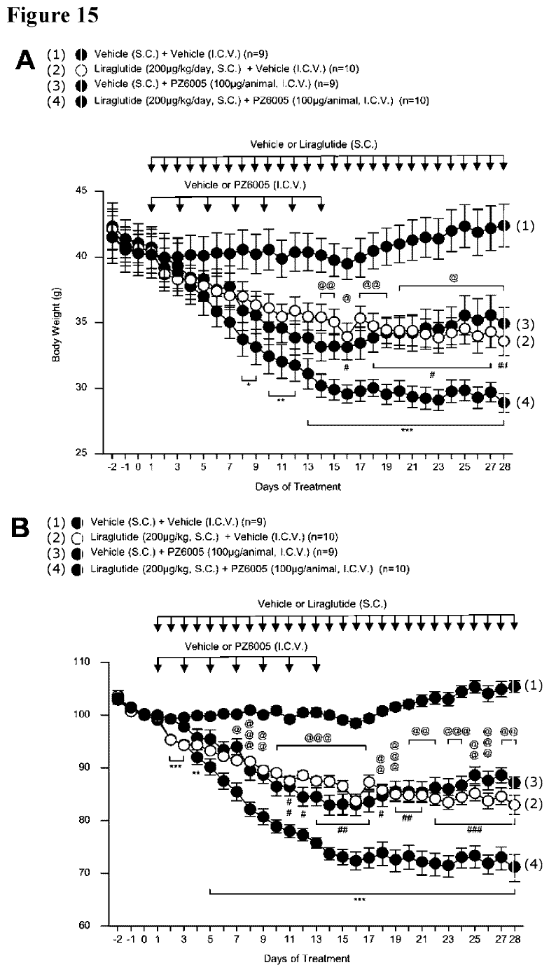

Mice treated with liraglutide alone showed enhanced ARC insulin receptor signalling compared with control littermates (FIG. 15a,b), consistent with a long-term liraglutide administration that could alleviate the loss of brain insulin receptors and restore neuronal insulin sensitivity. PZ6005, alone or co-administrated with liraglutide, provide an added improvement in insulin resistance, as assessed by more ARC pAKT +ve. cells observed in the mice receiving PZ6005 than those in the liraglutide alone group (FIG. 15a,b).

To determine whether PZ6005 enhances liraglutide actions on the ARC in diet-induced obesity, GLP-1 receptor signalling immunostained by anti-pERK, a marker of GLP-1 receptor activation, was evaluated (FIG. 16a). Compared to controls and liraglutide alone treatment, it was observed ARC pERK +ve. cells were markedly increased in the PZ6005 alone, or co-administered with liraglutide treatment (FIG. 16b). Moreover, PZ6005 is likely to improve the access of liraglutide to the ARC parenchyma through tanycytic transport, in keeping with hampered ARC liraglutide uptake in tanycytes-GLP-1-receptor knockout mice. The enhanced GLP-1 receptor signalling was accompanied by a robust increase in pERK +ve. cells inside the ARC and a mild but insignificant rising number of elongated ARC cells that lines the third ventricle (FIG. 16c,d).

These results demonstrate that pharmacologically abrogating neurofibrosis using PZ6005 enhances ARC insulin sensitivity, and it also allows tanycytes to transport more liraglutide to act on ARC parenchyma.

### Example 19. Co-Administration of Liraglutide with PZ6005 is Superior to Liraglutide and PZ6005 Monotherapy in Improving Whole-Body Metabolism in Diet-Induced Obesity

To determine the functional contribution of the co-administration of liraglutide with PZ6005 to whole-body metabolism, the influence on weight reduction was evaluated by measuring the weight of diet-induced obese mice receiving PZ6005 and/or liraglutide during the 28-day treatment. At day 28, the mean absolute weight change from baseline (day 0) of the PZ6005/liraglutide treatment group was −13.50±0.73 g than mice with vehicle treatment; −4.70±0.73 g than liraglutide treatment; and −6.04±0.73 g than PZ6005 treatment (FIG. 17a). The mean percentage change in weight was 5.22±1.08%, −17.22±1.84%, −13.02±1.15% and −28.98±2.57% in vehicle, liraglutide, PZ6005 and PZ6005/liraglutide treatment groups, respectively (FIG. 17b). Treatment with PZ6005 alone and liraglutide alone led to a decrease in body weight, whereas dual administration of PZ6005 and liraglutide significantly reduced weight more than the other three groups.

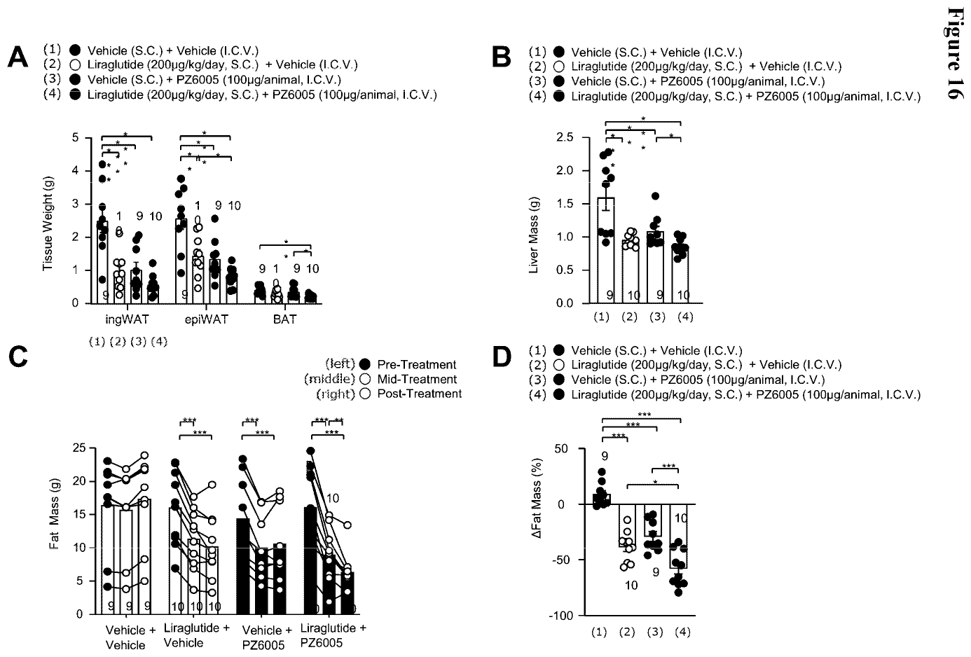

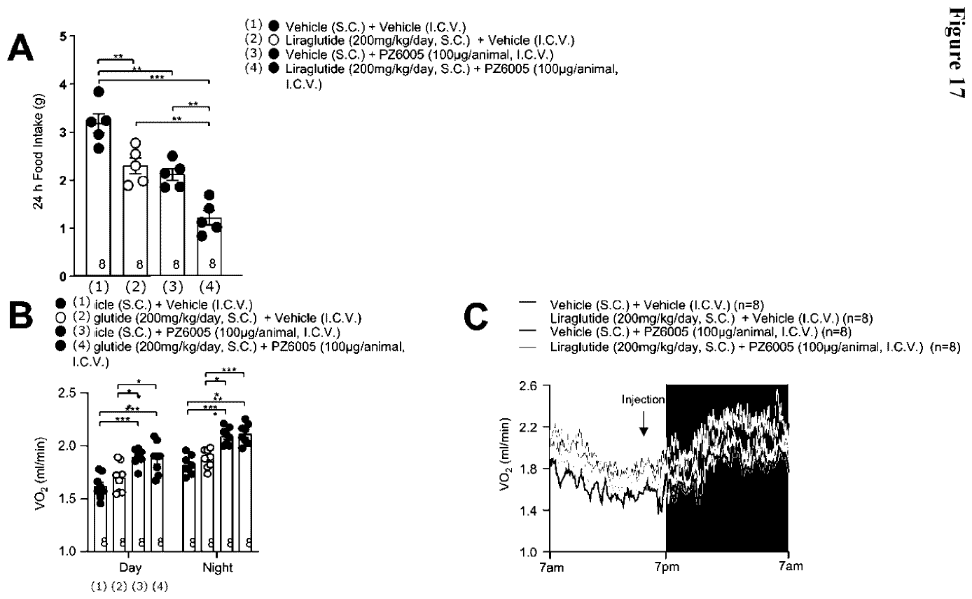

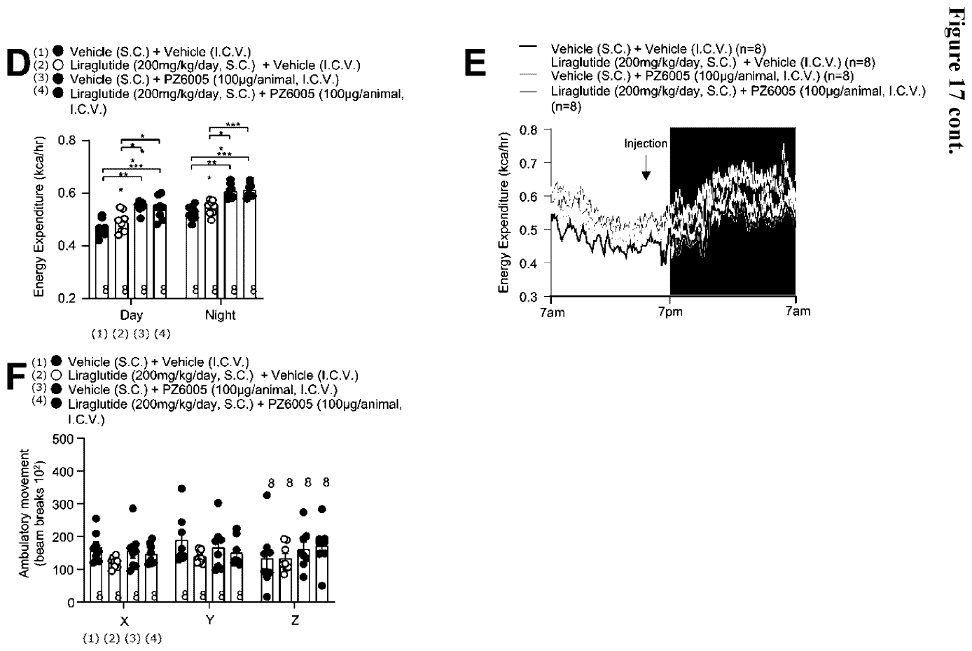

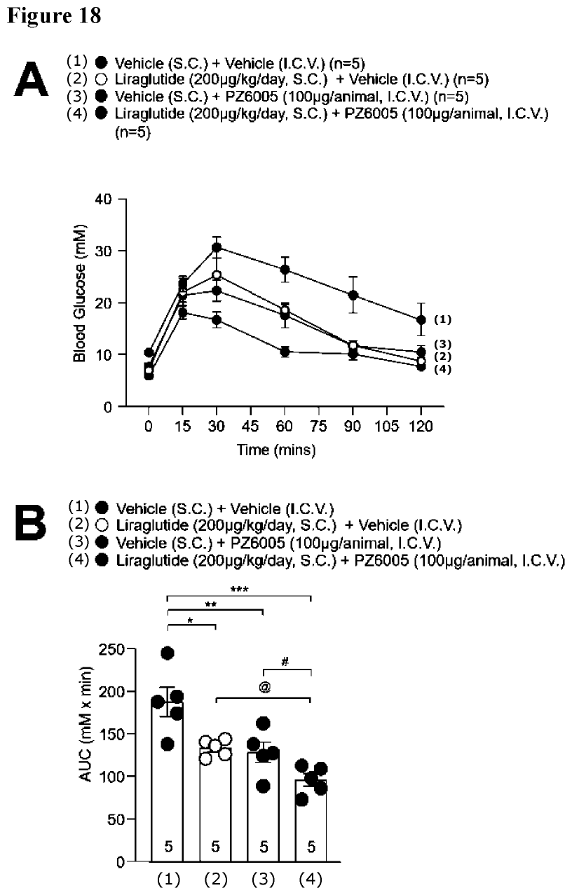

The influence of four treatments on altering tissue-specific adiposity and body composition was also compared. Compared with control littermates, mice in liraglutide treatment and PZ6005 treatment had a lower mass of ingWAT, epiWAT and the liver (FIG. 18a,b). PZ6005/liraglutide treatment reduced epiWAT more than liraglutide treatment, while the mice in this treatment showed less BAT and liver mass than the control treatment and PZ6005 treatment (FIG. 18a,b). Liraglutide/PZ6005 treatment was also superior to single-drug therapies for fat mass reduction, as assessed by a robust decrease in both mid- and post-treatment fat mass (FIG. 18c,d). The effects of PZ6005 on improving whole-body metabolism could be maintained for 14 days, as there was no significant difference in fat mass between the mid- and post-treatment in the PZ6005 treatment.

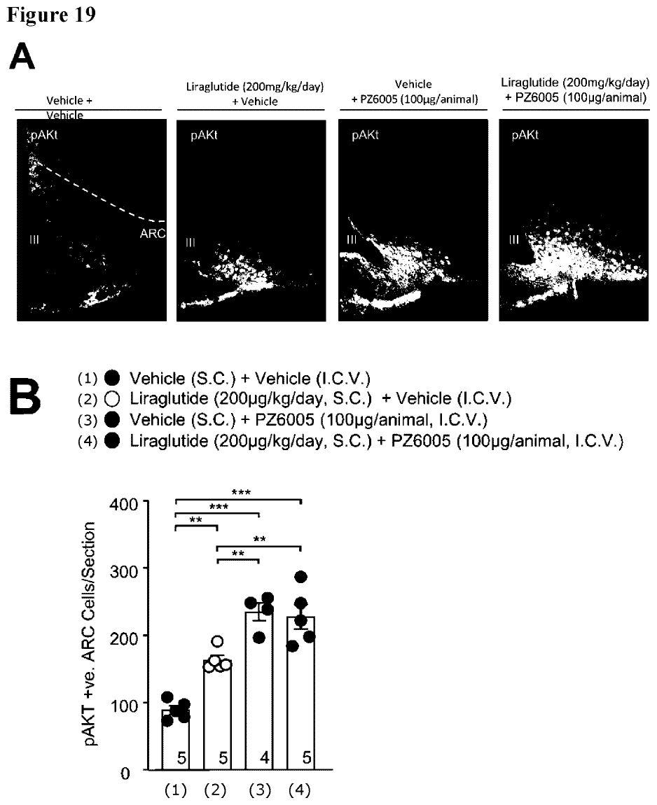

To further explore how PZ6005 enhances liraglutide-mediated weight loss, food intake and energy expenditure was assessed across 24 hours. Co-administration of liraglutide with PZ6005 significantly amplified the appetite-suppressing effect of two single therapies, as food intake in the dual therapy (co-administration of liraglutide and PZ6005) was lower than liraglutide and PZ6005 monotherapy group (FIG. 19a). Moreover, energy expenditure and oxygen consumption were unaffected by liraglutide treatment (FIG. 19b-e). During both day and night shifts, mice in both groups receiving PZ6005 consumed more energy without enhancing movement (FIG. 19b-e), in keeping with the improvement in whole-day energy expenditure observed in the I.C.V. PZ6005 experiment.

Taken together, these results suggest that dual administration of PZ6005 and liraglutide reduces appetite to achieve superior weight reduction compared to monotherapies in diet-induced obesity. This enhanced weight loss was not associated with any adverse effects such as sedation and hyperactivity and was accompanied by reduced stored fat and decreased epiWAT, BAT and liver mass.

### Example 20. Co-Administration of Liraglutide with PZ6005 is Superior to Liraglutide and PZ6005 Monotherapy in Improving Glycaemic Control in Diet-Induced Obesity

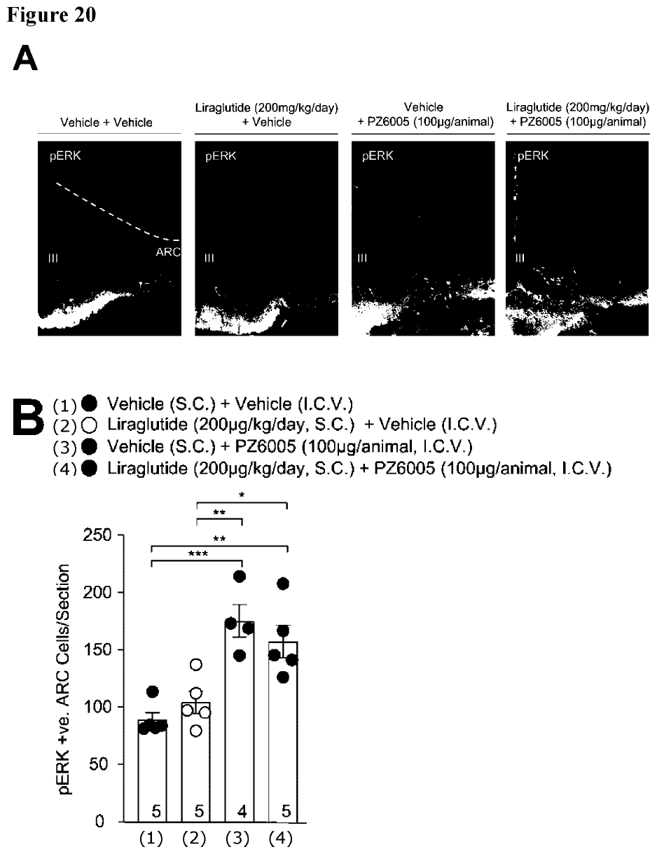

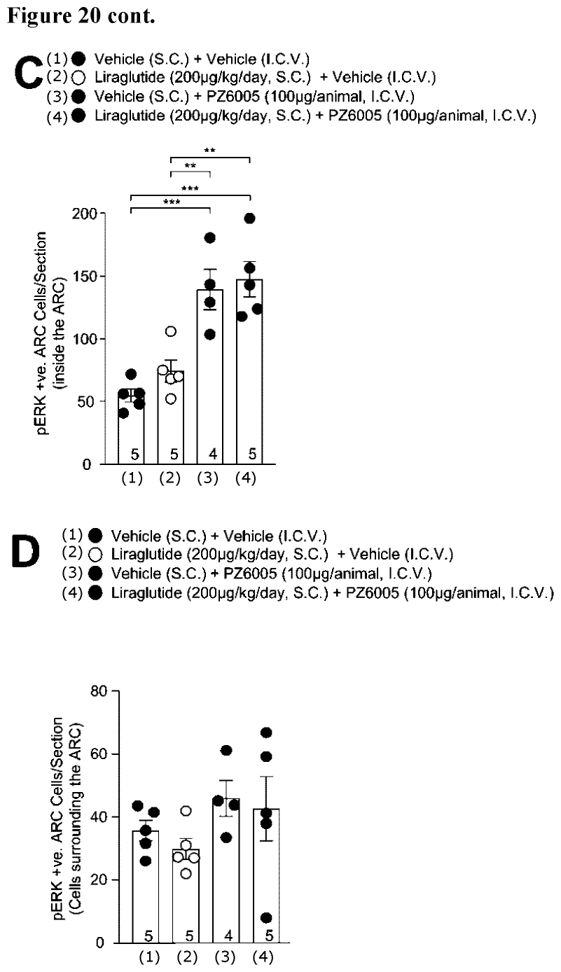

To demonstrate the influence of co-administration of liraglutide with PZ6005 on glycaemic control, obese mice administered vehicle or liraglutide and/or PZ6005, were fasted for 6 hours and then subjected to I.P. GTT. PZ6005 alone and liraglutide alone treatment reduced glucose excursion by similar extents compared with the control treatment (FIG. 3.4.4a,b). Liraglutide/PZ6005 treatment markedly lowered the peak blood glucose during the GTT, with the peak occurring 15 minutes earlier than the control group and two monotherapies (FIG. 20a). These results suggest that combination therapy of liraglutide and PZ6005 treatment provides an added beneficial glucose-lowering effect than liraglutide and PZ6005 treatment alone.

Taken together, these results demonstrate that dual delivery of liraglutide and PZ6005 has superior weight reduction and improvement in glycaemic control, over single liraglutide or PZ6005 treatment. Attenuated ARC neurofibrosis using PZ6005 was also accompanied by the enhancement of central GLP-1 signalling and the improvement of central insulin resistance. Moreover, these advantages of the combination therapy further spark interest in the therapeutic potential of targeting ARC for treating obesity and type-2 diabetes.

### Example 21. Neurofibrosis Inhibition Potentiates and Sustains the Capacity of GLP-1R Agonists to Promote the Remission of Obesity and Type-2 Diabetes

### Animals

All animal procedures were approved by The University of Melbourne Animal Ethics Committee (Ethics ID: 25349) and complied with the guidelines of the National Health and Medical Research Council for the use and care of research animals. C57BL/6J male mice were sourced from the Animal Resources Centre, Australia.

Eight-weeks-old C57BL/6J male mice were maintained on the 12-hour light/dark cycle in a temperature-controlled high-barrier facility with a room temperature (23±2° C.) and room humidity of 40%-70% per NHMRC Australian Code of Practice for the Care and Use of Animals. To induce diet-induced obesity in adulthood, the mice were ad libitum access to water and high-fat-high-sugar (HFHS) diet (43% of total energy from fat, SF04-001, Speciality Feeds, Australia) starting at eight weeks of age for 12 weeks. Aged-matched chow-fed C57BL/6J male mice were ad libitum access to standard chow diet throughout full duration of the experiment.

### Lateral Ventricular Cannulation

Under 2-5% (v/v) isoflurane in 1 L/min oxygen, 12-week-HFHS-diet-fed and aged-matched chow-fed C57BL/6J male mice were implanted stereotaxically with guide cannulas in the right lateral ventricle (measurement of the coordinates were described as Chapter 2; Anterior/Posterior: −1.00 mm, Medial/Lateral: +0.20 mm). Guide cannulas were made by polishing the shaft of hypodermic needles (19 Gauge, Livingstone, Australia) to 1.00 mm long. The tip of the guide cannula was positioned 1.00 mm above the target region (1.00 mm ventral to the surface of the mouse skull). The mice were allowed 2-3 days for recovery before further experimental manipulations. To assess the therapeutic potential of pharmacologically attenuated brain CSPG-ECM on prolonging anti-obesity and anti-diabetic effects of liraglutide, the mice were intracerebroventricularly (I.C.V.) administered with vehicle (ddH₂O) and PZ6005 (100 μg/animal) once every two days for 14-day treatment phase, in a total volume of 2 μl/animal at approximately 1 hr before lights off (7 pm).

### Intraperitoneal Injection

Awake 12-week-HFHS-diet-fed and aged-matched chow-fed C57BL/6J male mice were restrained by scruffing and inverted in the head-down position. 25 Gages syringe was injected into the lower right/left quadrant of mice abdomen, and the mice were then placed back into the house cage. Intraperitoneal administration of liraglutide (200 μg/kg of body weight, Novo Nordisk, Denmark) was daily performed at 6-7 pm prior to lights off during 14-day treatment phase.

### Metabolic Assessment

Metabolic measurements were undertaken in the Melbourne Mouse Metabolic Phenotyping Platform (The University of Melbourne, Australia). Mice adiposity and lean mass were measured via the TD-NMR minispec (Bruker Optics Inc., USA) acquiring EchoMRI-900 Body Composition Analyser (EchoMRI LLC, USA). Interscapular brown adipose tissue, inguinal and epidermal white adipose tissues and the liver were weighed to determine tissue-specific adiposity.

Mice metabolic parameters were assessed in the environmentally controlled monitoring cages at 5 minutes intervals for 48 hours after 24 hours of acclimation. Caloric intake was manually quantified by food consumption. Whole-body energy expenditure (VO2 consumption and energy expenditure) and locomotor activity were measured using Promethion Metabolic Screening System (Sable Systems International, USA) fitted with food consumption, indirect open circuit calorimetry, and activity monitors. Energy expenditure was calculated via the Weir equation (kcal h−1=60×(0.00394×VO2+0.001106×VCO2)). To account for the difference in energy expenditure based on body mass/composition was analysed and adjusted using ANCOVA analysis of the National Mouse Metabolic Phenotyping Centre Energy Expenditure Analysis Page (https://www.mmpc.org/shared/regression.aspx, USA). Fat and carbohydrates utilization were calculated as the ratio of VCO2 production over V02 consumption.

Glucose tolerance test was conducted on 6-hrs fasted conscious mice by injecting D-glucose (2 mg/g of lean body mass) into the peritoneal cavity and measuring blood glucose levels by taking tail blood immediately at 0, 15, 30, 60, 90 and 120 mins after injection using an Accu-Check glucometer (Roche, Germany). The area under the glucose excursion curve was determined and represented as mmol/L×min. Fasted (24 hr fast) glucose level was measured using an Accu-Check glucometer.

Insulin tolerance test was performed on 4-hrs fasted conscious mice by intraperitoneally injecting human insulin (0.6 mU/g of body weight for chow-fed mice and 0.8 mU/g of body weight for HSHF-fed obese mice, Actrapid, Novo Nordisk, Denmark) and measuring blood glucose level by taking tail blood immediately at 0, 15, 30, 45 and 90 mins after injection using an Accu-Check glucometer.

### Behavioural Satiety Sequence

12-week-HFHS-diet-fed and aged-matched chow-fed C57BL/6J male mice were fasted overnight and individually transferred into transparent cages with ad libitum access to water 3 hr before the observation. Five hours after the beginning of the light cycle (12 pm), food was presented to the mice, and mice were undisturbed and discreetly observed. Momentary behaviour was monitored every 30 seconds over a 90-minute observation. Behaviours were scored from 0 to 1 per the following classifications: Feeding (mice obtaining and chewing food), Drinking (mice trying to lick waterspout), Grooming (mice licking or scratching their bodies), Active (mice are moving and sniffing), Inactive (mice showing immobile or sickness signs when awake) and Resting (mice are sleeping). Data collected were collated into 5-minute period bins and used to analyse average percentages of time spent by animals for each type of behaviour (% of total behaviour), the transition from feeding to resting and the time to satiety.

### Results

To determine the functional contribution of co-administration of liraglutide with PZ6005 to postpone rebound effects in whole-body metabolism, metabolic measurements were conducted with 3 phases: 1) pre-treatment phase (day −2-0); 2) treatment phase (day 1-14) and 3) off-treatment phase (day 15-62).

12-week high-fat high-sugar (HFHS) fed obese C57BL/6j and aged-matched chow-fed mice were I.C.V. delivered with either vehicle or PZ6005 every second day; meanwhile, the mice received daily S.C. injections of either vehicle or liraglutide during the 14-day treatment phase. At day 15, treatments were discontinued however all groups continued to receive ad libitum access to HFHS diet to assess the rebound following treatment cessation.

During the treatment phase, the PZ6005/liraglutide group reduced body weight more than other treatment groups. At day 14, mean weight loss from baseline (day 0) was 22.97±2.39% with PZ6005/liraglutide, 15.72±1.32% with liraglutide and 15.08±1.45% with PZ6005 (FIG. 21a,b). After withdrawing treatments, the rate of weight regain was more decelerated in the PZ6005/liraglutide treated group compared with liraglutide treatment, demonstrating that PZ6005 prolongs the rebound on body weight observed following liraglutide treatment alone. Mice receiving PZ6005/liraglutide took 22.40±1.727 days to regain 50% of net weight loss, which was 14.5±1.75 days, 12.07±1.73 days and 2.73±1.55 days later than vehicle, liraglutide and PZ6005 arms, respectively (FIG. 21c). PZ6005/liraglutide group returned to baseline body weight at day 35.5±2.50 versus day 24.44±2.17 in vehicle group, day 28.00±3.08 in liraglutide group and day 30.00±1.72 in PZ6005 group (FIG. 21c). Treatment with liraglutide alone and PZ6005 alone took a similar time length to regain baseline weight as the vehicle, whereas dual administration of PZ6005 and liraglutide significantly prolongs the maintenance of weight-loss effects.

Corresponding to extended weight-reduction maintenance, PZ6005 alone and PZ6005/liraglutide treatment were superior in maintaining lean body mass and reduced whole-body adiposity. Compared to vehicle and liraglutide alone treatment groups, PZ6005 treated arms remained a significant decrease in baseline fat mass without affecting lean mass 14 days after terminating treatments (FIG. 21d-f). Mice receiving PZ6005 and PZ6005/liraglutide also had lower liver mass than other littermates with similar adiposity (FIG. 1e).

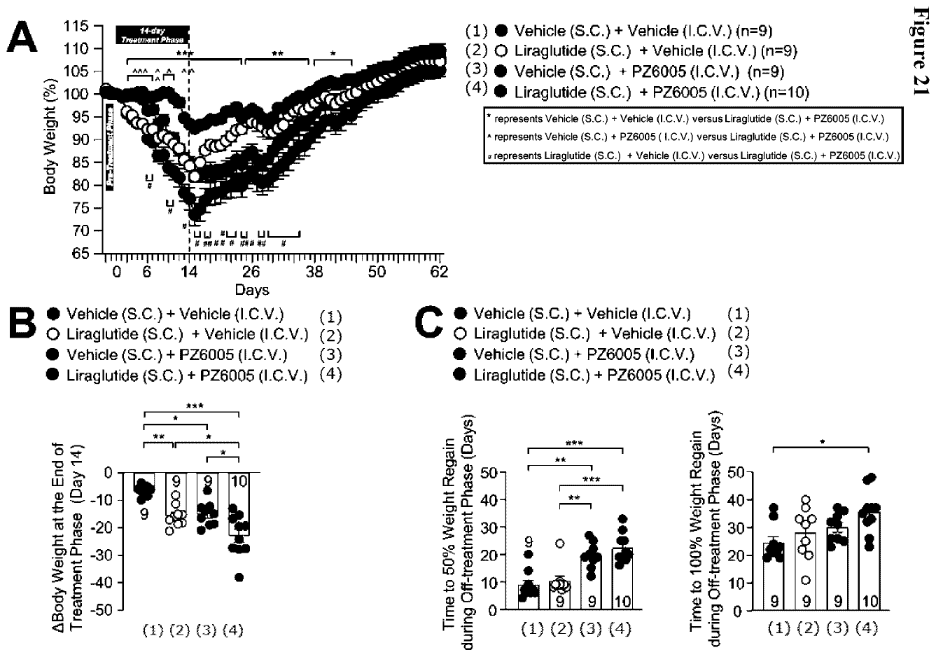

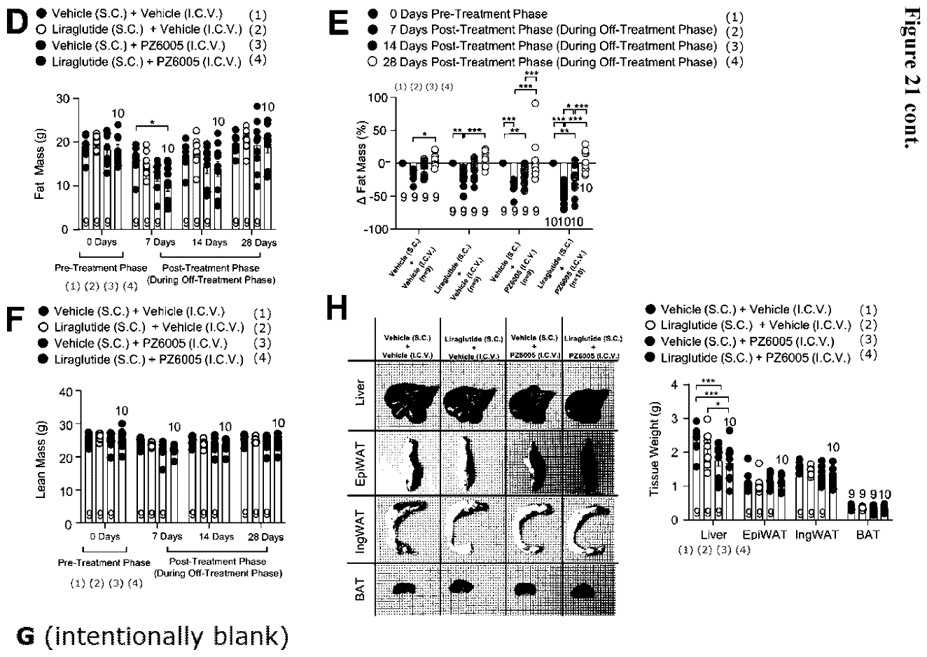

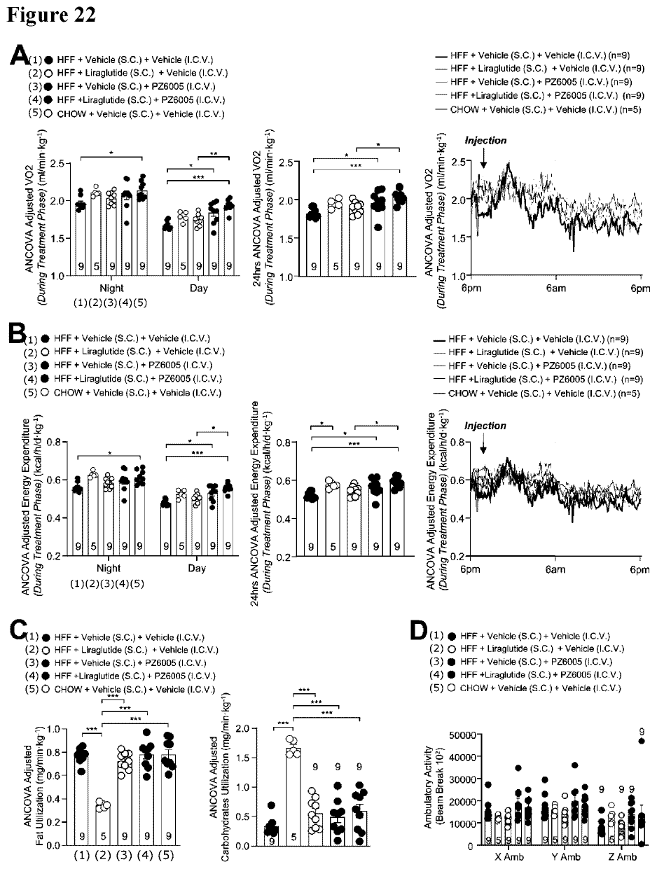

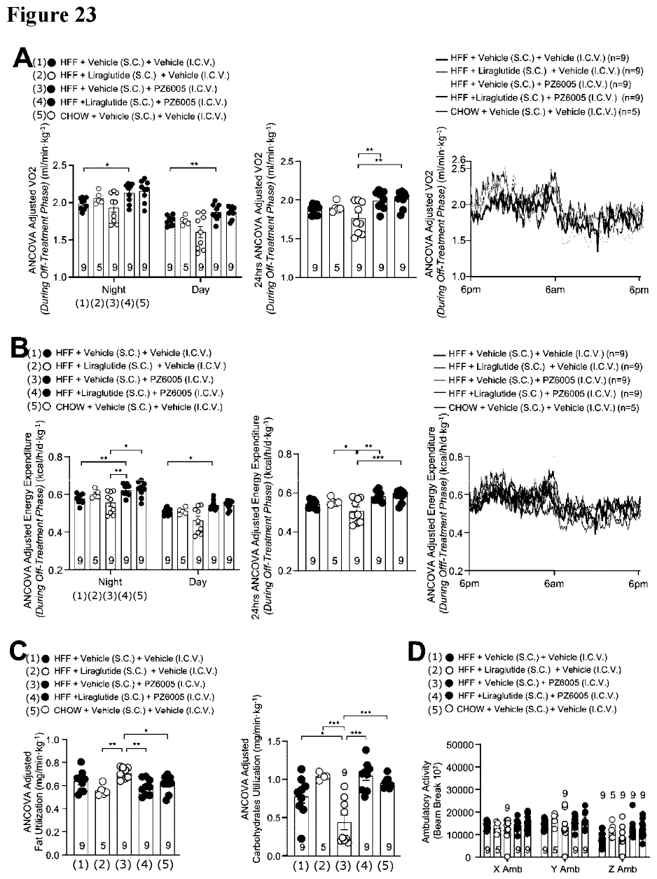

To explore the extent to which neurofibrosis inhibition contributes to the attenuation of rebound weight gain on liraglutide cessation, metabolic rate measurements of HFHS-diet-fed obese mice administered vehicle, liraglutide and/or PZ6005 and vehicle-treated chow-fed mice were assessed during treatment and off-treatment phase. Mice on PZ6005 alone and PZ6005/liraglutide treatment exhibited elevated oxygen consumption (FIG. 22a) and whole-body energy expenditure (FIG. 22b) without effects on ambulatory activity (FIG. 22e). Notwithstanding these effects, PZ6005/liraglutide administration did not statistically show a similar capacity of low-fat utilization and high-carbohydrate utilization to the healthy lean state during the treatment phase (FIG. 22c,d). Opposed to retained impacts of neurofibrosis attenuation on improving metabolic rates, mice with liraglutide monotherapy showed a reduction in oxygen consumption and energy expenditure with altered substrate utilization 14 days after withdrawing treatments (FIG. 23a-c). Specifically, cessation of liraglutide alone treatment led to a dramatic downregulation of carbohydrate utilization and increased fat utilization without signs of abnormal activities (FIG. 23c,d).

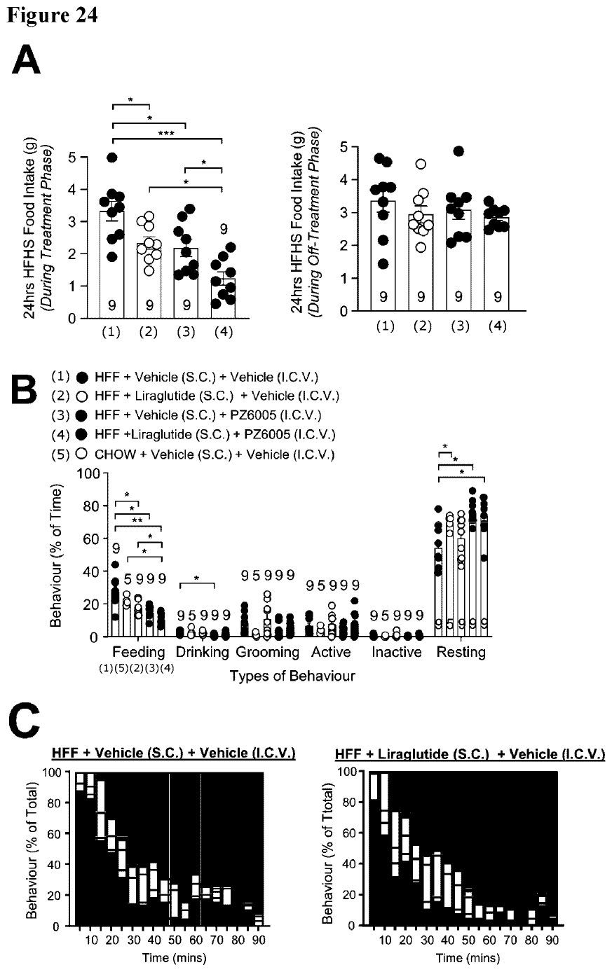

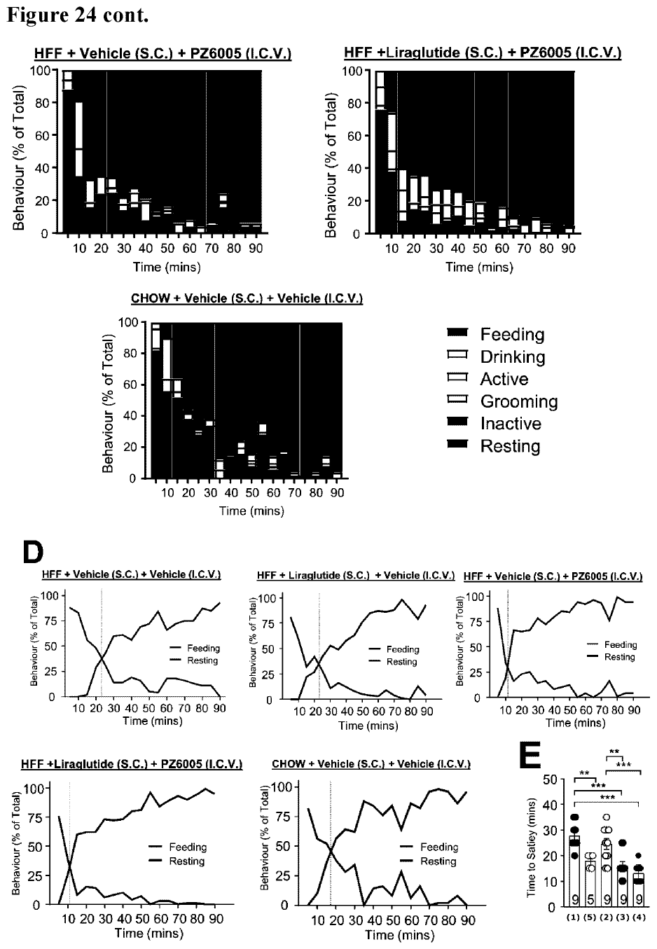

Attenuated weight rebound of PZ6005/liraglutide treatment was also accompanied by suppressed appetite, as assessed by reduced dietary intake. During treatment, mice with PZ6005/liraglutide consumed less high-fat-diet pallets than vehicle, liraglutide and PZ6005 alone arms (FIG. 24a). The advanced appetite-suppressing effect of the dual therapy was retained through suppressed appetite until 16 days after withdrawing treatment (FIG. 24a-d). This suggests that co-administration with PZ6005 prolongs the liraglutide-induced weight-loss effect through the maintenance of improved metabolic rates and reduced food intake.

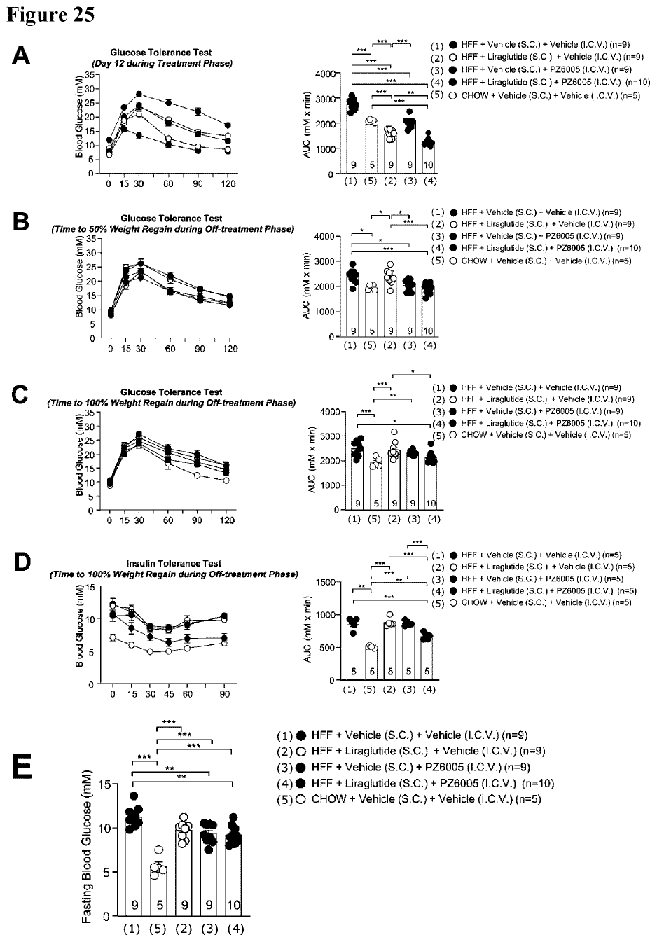

To determine the effects of co-administrating liraglutide with PZ6005 on the glycaemic rebound, the glycaemic control of HFHS or chow-fed mice treated with vehicle, liraglutide and/or PZ6005 was assessed at each treatment phase. Mice with liraglutide alone and PZ6005 alone had improved glucose tolerance, whereas mice with PZ6005/liraglutide dual therapy exhibited comparative glycaemic regulation to that of lean/healthy chow-fed mice during the treatment phase (FIG. 25a). Even after the PZ6005/liraglutide dual therapy group regained 50% or 100% of their original body weight, sustained improvements in glucose tolerance, fasting blood glucose and whole-body insulin sensitivity were observed (FIG. 5b-d). Notably, these assessments were conducted on separate days when the mice from each treatment group had regained ~50% and ~100% of overall weight loss. This indicates that PZ6005 lengthens the beneficial effects of liraglutide on remission from type-2 diabetes independent of any effects on body weight rebound.

### REFERENCES

Alonge, K. M., et al., Hypothalamic perineuronal net assembly is required for sustained diabetes remission induced by fibroblast growth factor 1 in rats. Nat Metab, 2020. 2(10): p. 1025-1033.
Anderson, E. M., et al., Systematic analysis of CRISPR-Cas9 mismatch tolerance reveals low levels of off-target activity. J Biotechnol, 2015. 211: p. 56-65.
Coskun, T., LY3437943, a novel triple glucagon, GIP, and GLP-1 receptor agonist for glycemic control and weight loss: From discovery to clinical proof of concept. Cell Metabolism, 2022. 24(9), 1234-1247.
Davies, M. J. et al., Efficacy of Liraglutide for Weight Loss Among Patients With Type 2 Diabetes: The SCALE Diabetes Randomized Clinical Trial. Jama, 2015. 314: 687-699.
Dodd, G. T., et al., TCPTP Regulates Insulin Signaling in AgRP Neurons to Coordinate Glucose Metabolism With Feeding. Diabetes, 2018. 67(7): p. 1246-1257.
Dodd, G. T., et al., Intranasal Targeting of Hypothalamic PTPIB and TCPTP Reinstates Leptin and Insulin Sensitivity and Promotes Weight Loss in Obesity. Cell Rep, 2019. 28(11): p. 2905-2922 e5.
Keough, M. B., et al., An inhibitor of chondroitin sulfate proteoglycan synthesis promotes central nervous system remyelination. Nat Commun, 2016. 7: p. 11312.
Mirzadeh, Z., et al., Perineuronal Net Formation during the Critical Period for Neuronal Maturation in the Hypothalamic Arcuate Nucleus. Nat Metab, 2019. 1(2): p. 212-221.
Moh, E. S. X., et al., Long-term intrathecal administration of morphine vs. baclofen: Differences in CSF glycoconjugate profiles using multiglycomics. Glycobiology, 2022. 32(1): p. 50-59.
Olofsson, L. E., et al., Modulation of AgRP-neuronal function by SOCS3 as an initiating event in diet-induced hypothalamic leptin resistance. Proc Natl Acad Sci USA. 2013. 110(8): p. E697-706.
Pi-Sunyer, X. et al. A Randomized, Controlled Trial of 3.0 mg of Liraglutide in Weight Management. N Engl J Med, 2015. 373: 11-22.
Stephenson, E. L., et al. Targeting the Chondroitin Sulfate Proteoglycans: Evaluating Fluorinated Glucosamines and Xylosides in Screens Pertinent to Multiple Sclerosis. ACS Cent Sci., 2019. 5(7): p. 1223-1234.
Winans, K. A. and Bertozzi, C. R. An Inhibitor of the Human UDP-GlcNAc 4-Epimerase Identified from a Uridine-Based Library: A Strategy to Inhibit O-Linked Glycosylation. Chemistry & Biology, 2022. 9(1): p. 113-129.

1. A pharmaceutical combination comprising (i) a 4-epimerase inhibitor and (ii) a GLP-1 receptor (GLP-1R) agonist.

2. The pharmaceutical combination of claim 1, wherein the 4-epimerase inhibitor is a fluorinated N-acetyl-glucosamine derivative, or a pharmaceutically acceptable salt, solvate or hydrate thereof, or a stereoisomer thereof.

3. The pharmaceutical combination of claim 1 or claim 2, wherein the 4-epimerase inhibitor is a compound of Formula (I): 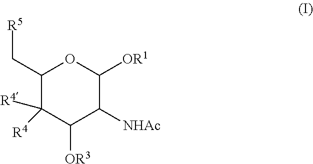
or a pharmaceutically acceptable salt, solvate or hydrate thereof, or a stereoisomer thereof,
wherein:
R¹, R³ and R⁵ are independently selected from H or C(O)C₁₋₄alkyl; and
R⁴ and R⁴′ are independently selected from H and fluoro, wherein at least one of R⁴ and R⁴′ is fluoro.

4. The pharmaceutical combination of any one of claims 1 to 3, wherein the 4-epimerase inhibitor is a compound of Formula (IA): 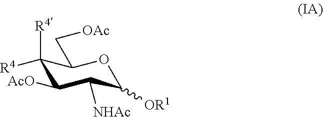
or a pharmaceutically acceptable salt, solvate or hydrate thereof, or a stereoisomer thereof,
wherein:
R¹, R³ and R⁵ are independently selected from H or C(O)C₁₋₄alkyl; and
R⁴ and R⁴′ are independently selected from H and fluoro, wherein at least one of R⁴ and R⁴′ is fluoro.

5. The pharmaceutical combination of claim 3 or claim 4, wherein R¹, R³ and R⁵ are independently selected from H or C(O)C₁₋₂alkyl.

6. The pharmaceutical combination of any one of claims 3 to 5, wherein R¹ is H or C(O)C₁₋₂alkyl, and R³ and R⁵ are both acyl groups.

7. The pharmaceutical combination of any one of claims 3 to 6, wherein R¹, R³ and R⁵ are each acyl groups.

8. The pharmaceutical combination of any one of claims 3 to 7, wherein the 4-epimerase inhibitor is selected from: 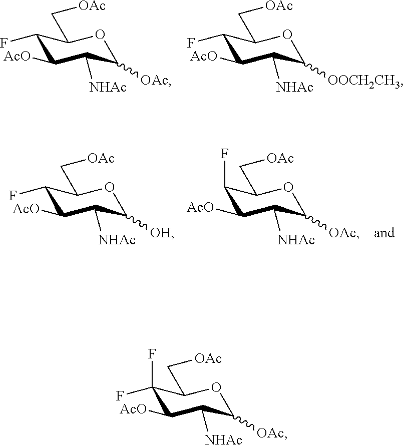
or a pharmaceutically acceptable salt, solvate or hydrate thereof, or a stereoisomer thereof.

9. The pharmaceutical combination of claim 8, wherein the 4-epimerase inhibitor is: 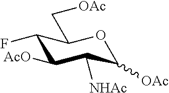
or a pharmaceutically acceptable salt, solvate or hydrate thereof, or a stereoisomer thereof.

10. The pharmaceutical combination of claim 1, wherein the 4-epimerase inhibitor is: 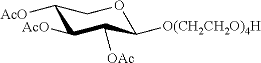
or a pharmaceutically acceptable salt, solvate or hydrate thereof, or a stereoisomer thereof.

11. The pharmaceutical combination of claim 1, wherein the 4-epimerase inhibitor is a compound of Formula (II), Formula (III) or Formula (IV): 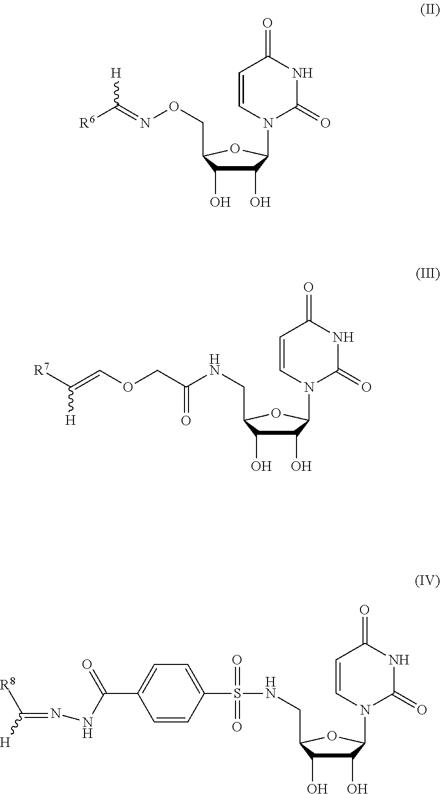
or a pharmaceutically acceptable salt, solvate or hydrate thereof, or a stereoisomer thereof,
wherein:
R⁶ is selected from: 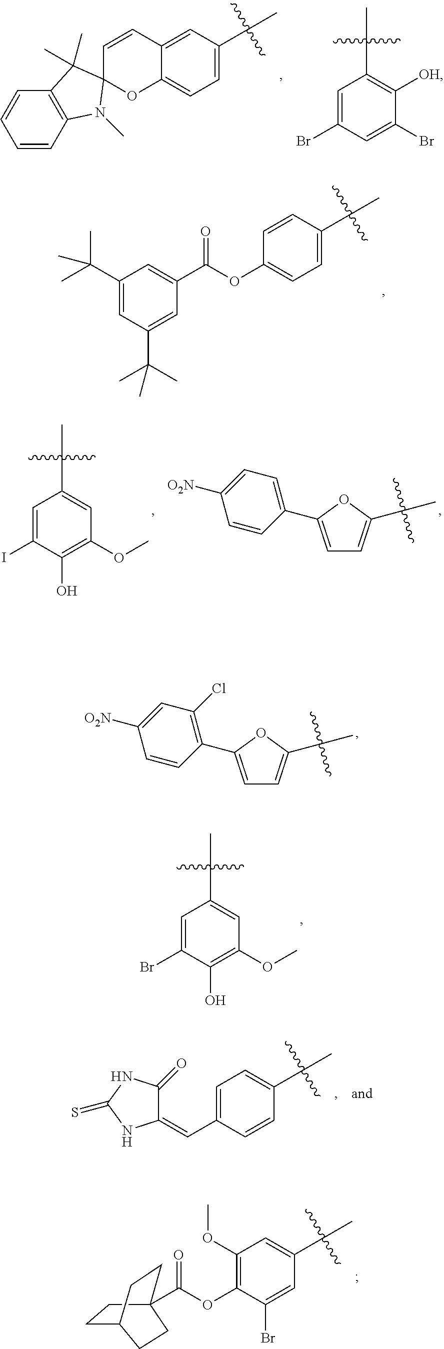
R⁷ is selected from: 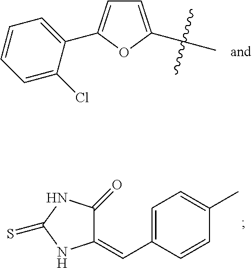
R⁸ is selected from:

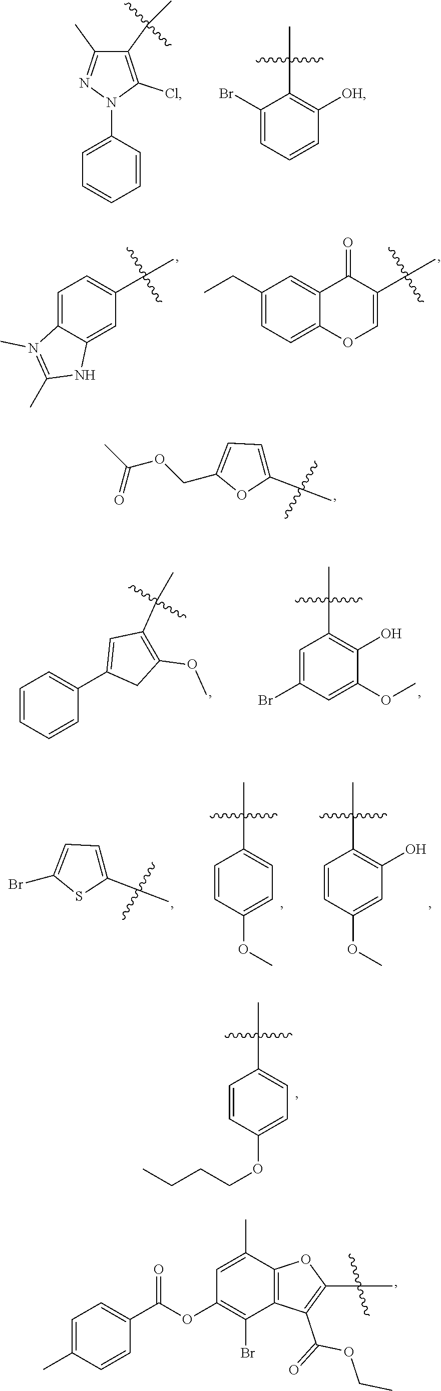

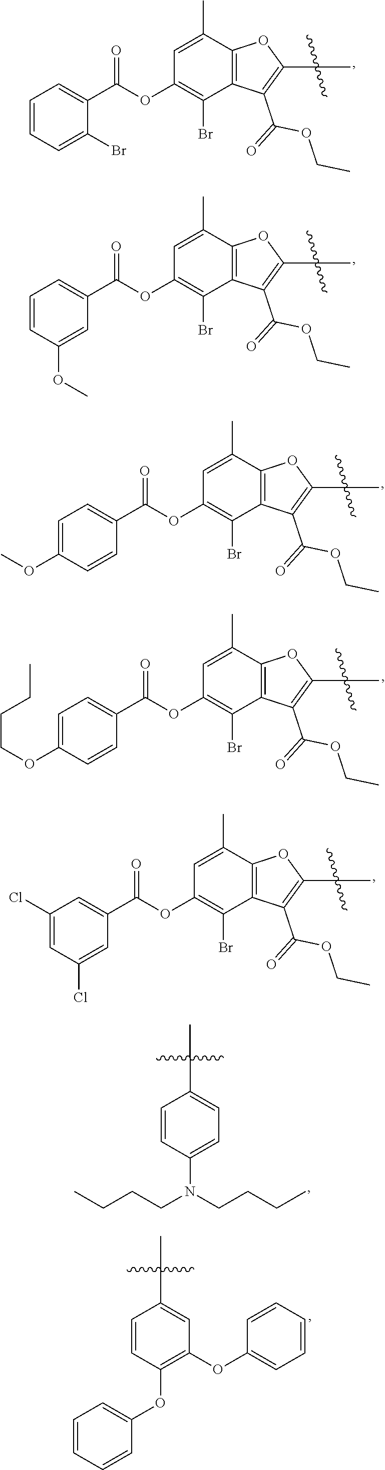

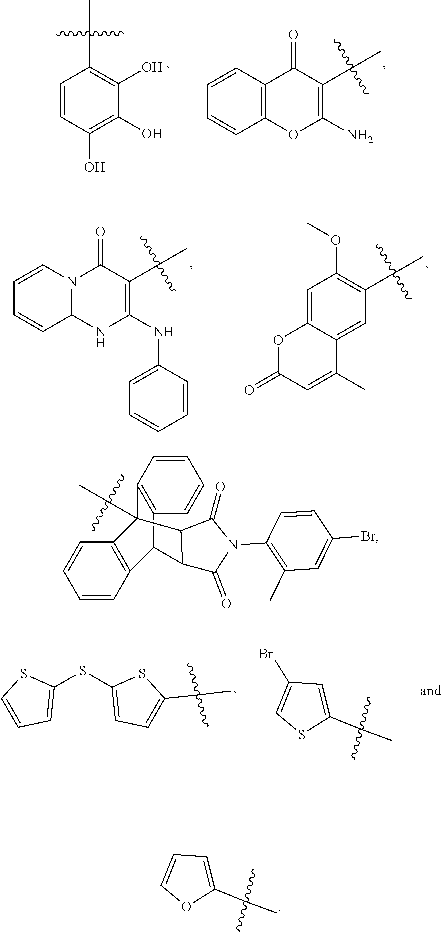

12. The pharmaceutical combination of any one of claims 1 to 11, wherein the GLP-1R agonist is GLP-1 or an analogue thereof.

13. The pharmaceutical combination of claim 13, wherein the GLP-1 analogue is selected from liraglutide dulaglutide, exenatide, semaglutide and lixisenatide, or any combination thereof.

14. The pharmaceutical combination of claim 12 or claim 13, wherein the GLP-1 analogue is liraglutide.

15. The pharmaceutical combination of any one of claims 1 to 11, wherein the GLP-1R agonist is a GLP-1R co-agonist.

16. The pharmaceutical combination of claim 15, wherein the GLP-1R co-agonist is tirzepatide or LY3437943.

17. A pharmaceutical composition comprising a 4-epimerase inhibitor and a GLP-1 agonist.

18. A method for treating or preventing insulin resistance or an associated disorder, supressing appetite and/or promoting weight loss in a subject, the method comprising administering to the subject an effective amount of a 4-epimerase inhibitor in combination with a GLP-1 receptor (GLP-1R) agonist.

19. The method of claim 18, wherein the associated disorder is selected from prediabetes, type-2 diabetes mellitus, obesity, metabolic syndrome, hypertension, dyslipidemia, atherosclerosis, non-alcoholic fatty liver disease (NAFLD), polycystic ovary syndrome (PCOS), coagulopathy and obstructive sleep apnoea.

20. The method of claim 18, wherein the associated disorder is a metabolic disease.

21. The method of claim 17, wherein the metabolic disease is type-2 diabetes mellitus.

22. The method of claim 20 for treating obesity, suppressing appetite and/or promoting weight loss.

23. The method of any one of claims 18 to 22, wherein the 4-epimerase inhibitor is administered intranasally.

24. The method of any one of claims 18 to 23, wherein the 4-epimerase inhibitor is administered every second day.

25. The method of any one of claims 18 to 24, wherein the GLP-1 agonist is administered by injection.

26. Use of a 4-epimerase inhibitor and a GLP-1R agonist in the manufacture of one or more medicaments for treating or preventing insulin resistance or an associated disorder, supressing appetite and/or promoting weight loss in a subject.

27. Use of a 4-epimerase inhibitor in the manufacture of a medicament for treating or preventing insulin resistance or an associated disorder, supressing appetite and/or promoting weight loss in a subject in combination with a GLP-1R agonist.

28. Use of a GLP-1R agonist in the manufacture of a medicament for treating or preventing insulin resistance or an associated disorder, supressing appetite and/or promoting weight loss in a subject in combination with a 4-epimerase inhibitor.

29. A combination comprising (i) a 4-epimerase inhibitor and (ii) a GLP-1R agonist, for use in treating or preventing insulin resistance or an associated disorder, supressing appetite and/or promoting weight loss in a subject.
